(19) United States
(12) Patent Application Publication
(10) Pub. No.: US 2012/0237954 A1
(43) Pub. Date: Sep. 20, 2012

(54) BIOMARKERS FOR PROGNOSES OF PULMONARY DISEASES

(75) Inventor: Steven R. Duncan, Pittsburgh, PA (US)

(73) Assignee: University of Pittsburgh - of the Commonwealth System of Higher Education, Pittsburgh, PA (US)

(21) Appl. No.: 13/432,227

(22) Filed: Mar. 28, 2012

Related U.S. Application Data
(63) Continuation of application No. PCT/US2010/052059, filed on Oct. 8, 2010.

(51) Int. Cl. G01N 21/64; C07K 16/28

(52) U.S. Cl. 435/7.24; 530/389.8

(57) ABSTRACT

The present invention relates to biomarkers that may be used to evaluate the prognoses of patients suffering from pulmonary diseases and assist in the determination of appropriate therapeutic regimens. It is based, at least in part, on the discovery that a number of T-cell antigens are differentially expressed in chronic lung disease patients depending on the prognosis of the patient. Non-limiting examples of these antigens include CD28, CD4, CD25, CD45, CD27 and CCR7 and combinations thereof. Use of these biomarker antigens, optionally in conjunction with pulmonary function tests, provides an indication of which patients are likely to suffer a severely adverse outcome within the year and/or be refractory to treatment.

### PRIORITY CLAIM

This application is a continuation of International Patent Application Serial No. PCT/US2010/052059 filed Oct. 8, 2010, and claims priority to U.S. Provisional Application Ser. No. 61/249,650 filed Oct. 8, 2009, the contents of both of which are hereby incorporated by reference in their entireties herein.

### GRANT INFORMATION

This invention was made with government support under Grant No. 1R01HL073241-01A3 awarded by the National Heart, Lung, and Blood Institute; Grant No. 1P50HL084948-01 awarded by the National Heart, Lung, and Blood Institute; and Grant No. 1 P50 HL084932-01 awarded by the National Institute of Health. The government has certain rights in the invention.

### 1. INTRODUCTION

The present invention relates to biomarkers that may be used to evaluate the prognoses of patients suffering from pulmonary diseases and assist in the determination of appropriate therapeutic regimens.

### 2. BACKGROUND OF THE INVENTION

Chronic lung disorders present a therapeutic challenge to clinicians. One factor that makes treatment difficult is being able to identify which patients will require aggressive measures. As background for the present invention, two examples of such chronic lung disorders are discussed below.

### 2.1 Lung Transplant Recipients Suffering Chronic Rejection

Chronic allograft rejection (CR) is the major limitation of success following transplantations of lungs and other solid organs. The prevalence of CR has not seemingly diminished over several decades, despite interval implementations of various immunosuppressants and other treatments (1. Boehler A, et al. Post-transplant bronchiolitis obliterans. Eur Respir J 2003; 22:1007-1018; Trulock E P, et al. Registry of the International Society for Heart and Lung Transplantation: twenty-third official adult lung and heart-lung transplantation report 2006. J Heart Lung Transplant 2006; 25:880-892; and Estenne M, et al. Bronchiolitis obliterans after human lung transplantation. Am J Respir Crit. Care Med 2002; 166:440-444). Fulminate CR in lung allograft recipients manifests with expiratory airflow obstruction, defined as bronchiolitis obliterans syndrome (BOS), as well as increased mortality. The natural history of this disorder is highly variable, however, and relatively quiescent courses that do not require or likely benefit from aggressive treatment are not uncommon (Nathan S D, et al. Bronchiolitis obliterans in single-lung transplant recipients. Chest 1995; 107:967-972).

Current therapies, with agents having nonspecific immunosuppressive effects, have uncertain efficacies and considerable toxicities. Better understandings of allograft pathogenesis could illustrate new approaches for clinical management of lung transplantation recipients. As an example, the ability to predict the likely progressiveness of CR in individual recipients could more productively direct surveillance and treatment to those at greatest risk, while limiting needless, and often substantial, side effects of immunosuppression augmentation among recipients destined for benign courses. In addition, the identification of a disproportionately pathogenic cell type could focus efforts to more specifically target or modify these particular cells.

Circulating CD4 T cells of lung allograft recipients with chronic rejection (“CR”) undergo abnormal oligoclonal expansions, as distinct from those cells from recipients with no evidence of chronic rejection (Duncan S R, et al., Oligoclonal CD4+ T cell expansions in lung transplantation recipients with obliterative bronchiolitis. Am J Respir Crit. Care Med 2002; 165:1439-1444). Further study has been focused on CD4 T-cell processes in lung allograft recipients, given the singular role of these lymphocytes in orchestrating adaptive immune responses, including allograft rejection (Krensky A M, et al. T-lymphocyte-antigen interactions in transplant rejection. N Engl J Med 1990; 322:510-517; Richards D M, et al. Indirect minor histocompatibility antigen presentation to allograft recipient cells in the draining lymph node leads to the activation and clonal expansions of CD4 T cells that cause obliterative airways disease. J Immunol 2004; 172:3469-3479; and Monaco C, et al. T-cell-mediated signaling in immune, inflammatory and angiogenic processes: the cascade of events leading to inflammatory diseases. Curr Drug Targets Inflamm Allergy 2004; 3:35-42).

### 2.2 Idiopathic Pulmonary Fibrosis

Idiopathic pulmonary fibrosis (IPF) is a chronic, progressive, and usually lethal fibrotic lung disease (Selman M., et al., Annals of internal medicine (2001) 134, 136-151). The disease is characterized by alveolar epithelial cell injury and activation, myofibroblast foci formation, and exaggerated accumulation of extracellular matrix in the lung parenchyma (Selman M., et al., Annals of internal medicine (2001); 134:136-151; American journal of respiratory and critical care medicine (2000); 161:646-664; Katzenstein A L and Myers J L American journal of respiratory and critical care medicine (1998); 157:1301-1315; Gross T J and Hunninghake G W, The New England journal of medicine (2000; 345:517-525). The incidence of IPF in the United States is 16.3 per 100,000 persons (Raghu G., et al. American journal of respiratory and critical care medicine (2006); 174:810-816) with a median survival of 2.5-3 years from diagnosis, and the mortality appears to be increasing (Olson A. L., et al. Am J Respir Crit. Care Med (2007); 176:277-284).

The etiology and molecular mechanisms underlying the lung phenotype in IPF are largely unknown. Although not widely considered to be an immunologic disorder (Selman M, Thannickal V J, Pardo A, Zisman D A, Martinez F J, et al. Idiopathic pulmonary fibrosis: pathogenesis and therapeutic approaches. Drugs (2004); 64: 406-430), several studies have shown that adaptive immune responses are often active in IPF patients, as evidenced by the frequent presence of IgG autoantibodies, increased productions of lymphocyte-derived inflammatory mediators, and abnormal extents of T-cell activation and clonal proliferations (Kurosu K, Takiguchi Y, Okada O, Yumoto N, Sakao S, et al. Identification of annexin 1 as a novel autoantigen in acute exacerbation of idiopathic pulmonary fibrosis. (2008); J Immunol 181: 756-767; Takahashi T, Wada I, Ohtsuka Y, Munakata M, Homm Y, et al. Autoantibody to alanyl-tRNA synthetase in patients with idiopathic pulmonary fibrosis. Respirology (2007); 12: 642-653; Magro C M, Waldman W J, Knight D A, Allen J N, Nadasdy T, et al. Idiopathic pulmonary fibrosis related to endothelial injury and antiendothelial cell antibodies. Hum Immunol (2006); 67: 284-297; Dobashi N, Fujita J, Murota M, Ohtsuki Y, Yamadori I, et al. Elevation of anti-cytokeratin 18 antibody and circulating cytokeratin 18: anti-cytokeratin 18 antibody immune complexes in sera of patients with idiopathic pulmonary fibrosis. (2000); Lung 178: 171-179; Wallace W A, Schofield J A, Lamb D, Howie S E Localization of a pulmonary autoantigen in cryptogenic fibrosing alveolitis. (1994); Thorax 49: 1139-1145; Pignatti P, Brunetti G, Moretto D, Yacoub M R, Fiori M, et al. Role of the chemokine receptors CXCR3 and CCR4 in human pulmonary fibrosis. Am J Respir Crit. Care Med (2006); 173: 310-317; Papiris S A, Kollintza A, Karatza M, Manali E D, Sotiropoulou C, et al. CD8+T lymphocytes in broncholaveolar lavage in idiopathic pulmonary fibrosis. J Inflamm (Lond) (2007); 4: 14-18; Marchal-Somme J, Uzunhan Y, Marchand-Adam S, Valeyre D, Soumelis V, et al. Cutting edge: non-proliferating mature immune cells form a novel type of organizing lymphoid structure in idiopathic pulmonary fibrosis. J Immunol (2006); 176: 5735-5739; Homolka J, Ziegenhagen M W, Gaede K I, Entzian P, Zissel G, et al. Systemic immune cell activation in a subgroup of patients with idiopathic pulmonary fibrosis. (2003); Respiration 70: 262-269; Shimizudani A, Murata H, Keino H, Kojo S, Nakamura H, et al. Conserved CDR 3 region of T cell receptor BV gene in lymphocytes from bronchoalveolar lavage fluid of patients with idiopathic pulmonary fibrosis. Clin Exp Immunol (2002); 129: 140-149; Feghali-Bostwick C A, Tsai C G, Valentine V G, Kantrow S, Stoner M W, et al. Cellular and humoral autoreactivity in idiopathic pulmonary fibrosis. J Immunol (2007); 179: 2592-9). Activated CD4 T-cells may also infiltrate into IPF lungs prior to development of symptoms (Rosas I O, Ren P, Avila N A, Chow C K, Franks T J, et al. Early interstitial lung disease in familial pulmonary fibrosis. Am J Resp Crit. Care Med (2007); 176: 698-705), and pro-inflammatory dendritic cells accumulate in the pulmonary parenchyma of advanced cases (Marchal-Somme J, Uzunhan Y, Marchand-Adam S, Kambouchner K, Valeyre D, et al. Dendritic cells accumulate in human fibrotic interstitial lung disease. Am J Respir Crit. Care Med (2007); 176: 1007-1014). FoxP3+ regulatory T-cells (Treg), widely regarded as having important roles in dampening adaptive immune responses against antigens (Sakaguchi S, Naturally arising CD4+ regulatory t cells for immunologic self-tolerance and negative control of immune responses. Annu Rev Immunol (2004); 22: 531-562), are diminished in both the circulation and broncholavolar lavage returns of IPF patients (Kotslanidis I, Nakou E, Bouchliou I, Tzouvelekis A, Spanoudakis E, et al. Global impairment of CD4+CD25+FoxP3+ regulatory T cells in idiopathic pulmonary fibrosis. Am J Resp Crit. Care Med (2009); 179: 1121-1130). More directly compelling, a protein(s) uniquely present in IPF lungs induces proliferation of autologous CD4 T-cells from these patients (Feghali-Bostwick C A, Tsai C G, Valentine V G, Kantrow S, Stoner M W, et al. Cellular and humoral autoreactivity in idiopathic pulmonary fibrosis. J Immunol (2007); 179: 2592-9).

### 2.3 CD28

CD28, a costimulatory molecule, is often down-regulated on T cells of patients with chronic adaptive immune diseases, and the unusual CD4 T cells that do not express CD28 (CD4+CD28null) have also been implicated in the immunopathogenesis of these disorders (see, e.g., Vallejo A N, et al. T-cell senescence: a culprit of immune abnormalities in chronic inflammation and persistent infection. Trends Mol Med 2004; 10:119-124; Vallejo A N. CD28 extinction in human T cells: altered functions and the program of T-cell senescence. Immunol Rev 2005; 205:158-169; van Leeuwen E M M, et al. Emergence of a CD4+CD28− granzyme B1 cytomegalovirusspecific T cell subset after recovery of primary cytomegalovirus infection. J Immunol 2004; 173:1834-1841; Goronzy J J, et al. Prognostic markers of radiographic progression in early rheumatoid arthritis. Arthritis Rheum 2004; 50:43-54; Raffeiner B, et al. Between adaptive and innate immunity: TLR4-mediated perforin production by CD28null T-helper cells in ankylosing spondylitis. Arthritis Res Ther 2005; 7: R1312-R1320; Zal B, et al. Heat-shock protein 60-reactive CD4+CD28null T cells in patients with acute coronary syndromes. Circulation 2004; 109:1230-1235; Lamprecht P, et al. CD28 negative T cells are enriched in granulomatous lesions of the respiratory tract in Wegener's granulomatosis. Thorax 2001; 56: 751-757; Komocsi A, et al. Peripheral blood and granuloma CD4+CD28− T cells are a major source of interferon-g and tumor necrosis factor-a in Wegener's granulomatosis. Am J Pathol 2002; 160:1717-1724; Snyder M R, et al. Formation of the killer IgG-like receptor repertoire on CD4+CD28null T-cells. J Immunol 2002; 168; 3839-3846; Liuzzo G, et al. Monoclonal T-cell proliferation and plaque instability in acute coronary syndromes. Circulation 2000; 101:2883-2888; Hirokawa M, et al. Oligoclonal expansion of CD4+CD28− T lymphocytes in recipients of allogeneic hematopoietic cell grafts and identification of the same T cell clones within both CD4+CD28+ and CD4+CD28− T cell subsets. Bone Marrow Transplant 2001; 27:1095-1100; Schirmer M, et al. Resistance to apoptosis and elevated expression of Bcl-2 in clonally expanded CD4+CD28− T cells from rheumatoid arthritis patients. J Immunol 1998; 161:1018-1025; and Pawlik A, et al. The expansion of CD4+CD28− T cells in patients with chronic kidney graft rejection. Transplant Proc 2003; 35:2902-2904).

Idiopathic pulmonary fibrosis (IPF) is a chronic fibroproliferative lung disease that afflicts ˜40,000 patients in the U.S. each year [1]. IPF typically manifests with inexorable pulmonary restriction and hypoxemia, resulting in progressive exercise limitation and dyspnea. The disease has a grim prognosis, with a median survival of ˜3 years after diagnosis, although courses can be highly variable. No medical treatments have yet been shown to alter the natural history of this disease [1], [2].

### 3. SUMMARY OF THE INVENTION

The present invention relates to biomarkers that may be used to evaluate the prognoses of patients suffering from pulmonary diseases and assist in the determination of appropriate therapeutic regimens. It is based, at least in part, on the discovery that a number of T-cell antigens are differentially expressed in chronic lung disease patients depending on the prognosis of the patient. Non-limiting examples of these antigens include CD28, CD4, CD25, CD45RO, CD27 and CCR7 and combinations thereof. Use of these biomarker antigens, optionally in conjunction with pulmonary function tests, provides an indication of which patients are likely to suffer a severly adverse outcome within the year and/or be refractory to treatment.

### 3.1 DEFINITIONS

Slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases. “About” means plus or minus 10 percent.

By “outcome” it is meant a clinical end-point, such as, without limitation death, restoration of health, or the presence or lack of one or more symptom or marker indicative of the status of a subjects health, such as status of a disease or condition, or death, or the need for artificial respiration, or a surgical procedure such as lung transplant.

In relation to the prevalence or proportion of a given marker(s) on a cell population and its correlation with a condition, disease or phenotype—by “abnormally high” or “abnormally low”, it is meant that the prevalence or proportion of the marker(s) on or in cells obtained from a statistically significant population of individuals having the condition, disease or phenotype is higher or lower as compared to the prevalence or proportion of the marker(s) on or in a cell or cells obtained from a statistically significant population of healthy individuals, such that the prevalence or proportion of the marker(s) on cells obtained from an individual can serve as a predictor of a clinical outcome in that individual that is related to the condition, disease or phenotype. The ability of the abnormally high or low prevalence or proportion of cells exhibiting the marker to predict a clinical outcome need not be absolute, but it cannot be random and preferably is predictive to a degree acceptable in the medical, forensic and/or pathology arts. The marker may be any detectable cellular (intra- or extra-cellular) component, constituent, antigen, epitope, receptor, ligand, etc. Statistical significance refers to any acceptable and useful statistical function, distribution, equation, etc., so long as it is acceptable in the medical, forensic and/or pathology arts.

Immunosuppressive drugs, immunosuppressive agents, or immunosuppressants are drugs that inhibit or prevent activity of the immune system. They are used in immunosuppressive therapy to: Prevent the rejection of transplanted organs and tissues; treat autoimmune diseases or diseases that are most likely of autoimmune origin (e.g., rheumatoid arthritis, multiple sclerosis, myasthenia gravis, systemic lupus erythematosus, Crohn's disease, pemphigus, and ulcerative colitis); or treat some other non-autoimmune inflammatory diseases (e.g., long term allergic asthma control). Non-limiting examples of immunosuppressive drugs include glucocorticoids (e.g., hydrocortisone, prednisone, prednisolone, etc.), cytostatics, such as alkylating agents including nitrogen mustads and platinum compounds; antimetabolites such as nucleoside analogs; metotrexate etc.); antibodies, such as anti-IL-2 and anti-CD3 antibodies; drugs acting on immunophilins such as cylosporin, tacrolimus, etc.; and other drugs such as interferons, TNF-binding proteins, etc.

A “transplant” is a graft, including, without limitation an autograft (self), an isograft (a genetically identical donor), an allograft (intraspecies) and a xenograft (interspecies). A graft or transplant may comprise synthetic, man-made, components, such as polymeric cell growth scaffolding, and/or natural extracellular matrix-derived components along with autogenic, isogenic, allogenic or xenogenic tissues, cells, etc. A “transplant patient” is a human, mammal or other animal in receipt of a transplant.

Chronic obstructive pulmonary disease (COPD) is a disease of the lungs in which the airways become narrowed. This leads to a limitation of the flow of air to and from the lungs causing shortness of breath. In contrast to asthma, the limitation of airflow is poorly reversible and usually gradually gets worse over time. Chronic bronchitis and emphysema are types of COPD. COPD has an inflammatory component and may be caused by inhalation of noxious gases or particles, as in the case of smoking, or graft rejection.

The following is an explanation of common test values in spirometry tests for lung function

Forced Vital Capacity (FVC) is the total amount of air that can forcibly be blown out during (or with) full inspiration, measured in liters.

FVC%p is the percentage of normal predicted FVC.

Forced Expiratory Volume in 1 Second (FEV1) is the amount of air that you can forcibly blow out in one second, measured in liters. Along with FVC it is considered one of the primary indicators of lung function.

FEV1% (FEV1/FVC) is the ratio of FEV1 to FVC. In healthy adults this should be approximately 75-80%. Absolute value of “normal” is >/=70.

Peak Expiratory Flow (PEF) is the speed of the air moving out of your lungs at the beginning of the expiration, measured in liters per second.

Forced Expiratory Flow 25-75% or 25-50% (FEF 25-75% or 25-50%) is the average flow (or speed) of air coming out of the lung during the middle portion of the expiration (also sometimes referred to as the MMEF, for maximal mid-expiratory flow).

Forced Inspiratory Flow 25%-75% or 25%-50% (FIF 25-75% or 25-50%) is similar to FEF 25%-75% or 25%-50% except the measurement is taken during inspiration.

Forced Expiratory Time (FET) measures the length of the expiration in seconds.

Tidal Volume (TV)—during the respiratory cycle, a specific volume of air is drawn into and then expired out of the lungs. This volume is tidal volume.

Maximum Voluntary Ventilation (MVV) is a measure of the maximum amount of air that can be inhaled and exhaled in one minute, measured in liters/minute.

Functional residual capacity (FRC), the volume of air present in the lungs at the end of passive expiration, cannot be measured via spirometry, but it can be measured with a plethysmograph.

Results for these assays are usually given in both raw data (liters, liters per second) and percent predicted—the test result as a percent of the “predicted values” for the patients of similar characteristics (height, age, sex, and sometimes race and weight). The interpretation of the results can vary depending on the physician and the source of the predicted values. Generally speaking, results nearest to 100% predicted are the most normal, and results over 80% are often considered normal.

The lung carbon monoxide diffusing capacity (DLCO) test determines how effectively gases are exchanged between the blood and airways in the lungs. In this assay, a patient inhales a mixture of carbon monoxide, helium, and oxygen and holds his or her breath for about 10 seconds. The gas levels are then analyzed from the exhaled breath.

### 4. BRIEF DESCRIPTION OF THE FIGURES

FIG. 1A-D. Characteristics of CD4 T-cell subpopulations in lung allograft recipients. (A and B) Flow cytometry methodology. (A) T cells that stained brightly with anti-CD4 monoclonal antibody conjugated to allophycocyanin and CD3 conjugated to Cy-Chrome were gated for subsequent determinations of cellular expressions. (B) Expressions of other phenotypic markers, in this example denoted by anti-CD25 phycoerythrin (PE) antibody staining, were individually determined in autologous CD4+CD28+ and CD4+CD28null subpopulations. In the example here, these respective subpopulation are denoted by the presence or absence, respectively, of costaining with anti-CD28 antibody conjugated to fluorescein isothiocyanate (FITC). (C) The percentages of circulating CD4 T cells that also expressed CD28 (CD28%) were reduced in lung transplant recipients with bronchiolitis obliterans syndrome (BOS) in comparisons with healthy normal (nontransplanted) control subjects. Horizontal lines denote population means. The No-BOS recipient with a CD28% of 67.4 had obliterative bronchiolitis on lung biopsy, but normal expiratory flow at the time of this CD4 assay. (D) In contrast to autologous CD4+CD28+, the CD4+CD28null T cells from lung transplant recipients with BOS less often express activation marker CD25 (n 5 16). Assays in later cohorts of consecutive, randomly selected subjects also show that the CD4+CD28null cells from recipients with BOS much more frequently produce the cytoxic mediators, perforin and granzyme B (both n=10), but less often express FoxP3+(n=6), in comparisons to autologous CD4+CD28+ cells.

FIG. 2. Nonparametric Receiver Operative Characteristics (ROC) curve for CD4+CD28+/total CD4 (CD28%) determinations, as a clinical predictive test for BOS among the initial subject population (Cohort A). The ROC is plotted between the true positive rate (sensitivity) on the y-axis, and the false positive rate (1-specificity) on the x-axis. Area under curve (AUC) represents the accuracy of the CD28% and was 0.67 (standard error 0.12, 95% C.I. 0.44-0.90).

FIG. 3. Cytokine elaborations by autologous CD4 subpopulations of recipients with chronic rejection. The initial (left) data point in each series represents control unstimulated (US) condition, whereas the second (right) data point delineates production of cells after stimulation with plate-bound anti-CD3 antibody (Stim). These paired specimens (US and Stim) are also connected by lines. CD4+CD28null cells from transplant recipients with BOS (open circles) tend to elaborate greater amounts of proinflammatory and Th1 cytokines (top two rows), whereas CD4+CD28+ (open squares with paired specimens connected by solid lines) have an apparent Th2 bias (bottom row) (n=5 randomly selected, consecutive specimens in each). G-CSF=granulocyte colony-stimulating factor; MIP-1b 5 macrophage inflammatory protein-1b.

FIG. 4A-C. Effects of cyclosporine A on proliferation of CD4 T-cell subpopulations isolated from lung transplant recipients with chronic rejection. (4A) Illustrative example of proliferation quantitation as assessed by bromodeoxyuridine (BrdU) incorporation and flow cytometry. Viable cells were gated and respective populations of CD4 T cells (in this case CD4+CD28null) that incorporated BrdU were determined. In this example, ˜83% of the CD4+CD28null incorporated BrdU. All CD4+ cells had been previously depleted from these cultures, except the particular CD4 T-cell subpopulation being evaluated (either CD28+ or CD28null) (see METHODS). (4B) Proliferations, ascertained by the percentages of respective autologous CD4 T-cell subpopulations that incorporated BrdU after stimulation by plate-bound anti-CD3 antibody, were approximately equivalent in both CD4 T-cell groups in the absence of cyclosporine (control). In every recipient with BOS tested (n=6), however, proliferation among their CD4+CD28+ cells were decreased more by cyclosporine (100 ng/ml) than were the proliferations of their autologous CD4+CD28null cells. (4C) The aggregate decrement of proliferation in the presence of cyclosporine, relative to control (no cyclosporine) values, was significantly greater among the CD4+CD28+ cells. APC=allopycocyanin; CsA=cyclosporine A.

FIG. 5. Graph showing decrements of FEV1 were significantly greater among cohort A recipients with CD4+CD28+/CD4total values less than 0.9 (CD28% Low; n=7) compared with recipients with CD4+CD28+/CD4total values of 0.9 or greater (CD28% High; n=27). Routine surveillance pulmonary function tests (PFTs) were performed 6.0 (SEM, ±0.5) months and 6.6 (SEM, ±1.3) months after CD28 determinations (CD28% Low and CD28% High, respectively). One CD28% Low subject did not have pulmonary function measured after her initial CD4 assay due to severe allograft dysfunction (BOS) and later demise.

FIG. 6A-B. (A) Subsequent decrements of FEV1 were significantly greater among Cohort A CD28% Low with BOS compared to the CD28% High recipients with BOS. Pulmonary function was measured at routine, surveillance evaluations 6 months after their CD4 assays (see also the brief description of FIG. 5A). (B) Decrements of FEV1 remained significantly greater among Cohort A CD28% Low (n=5) compared to CD28% High (n=22) at 11.8+0.7 and 11.9+0.4 months after their initial CD28 measurement, respectively. Subject drop out in both populations was due to interval deaths or development of severe graft dysfunction that precluded these later pulmonary function tests.

FIG. 7. Changes in CD28 expression by CD4 T-cells of recipients (CD28%) over time, among 14 Cohort A subjects with replicate assays. Months denote intervals between first and second CD28 determinations. Open squares denote values of recipients without BOS at the time of these assays, whereas open circles denote subjects with BOS at these respective assay time points. Nine of these recipients were No-BOS at the time of their initial assays, but seven of these developed BOS prior to their second CD28% measurements.

FIG. 8A-C. (A) Association of changes in FEV 1 (as percentages of initial values) versus changes in CD28% among those cohort A subjects who were available and consented to replicate studies. Open squares denote those recipients who were non-BOS at the time of their first T-cell assay, but had progressed to BOS by the time of their second determination; closed squares denote those recipients who had BOS at both CD28% determinations; and open circles represent recipients who were non-BOS throughout. (B) Survival curves showing cumulative freedom from major adverse events of CD28% High (n=46) and CD28% Low (n=19) among all recipients in the aggregate subject populations (both cohorts A and B). Tick marks denote interval-censored events, and numbers in parentheses at end of the survival curves denote remaining, unafflicted subjects who were censored at 24 months of observation. (C) Survival curves showing cumulative freedom from major adverse events of CD28% High (n=24) and CD28% Low (n=16) among all BOS recipients in the aggregate subject populations (both cohorts A and B).

FIG. 9A-C. (A) Actuarial survival curves showing cumulative freedom from major adverse events (retransplantation or death) of CD28% High (n=27) and CD28% Low (n=8) among Cohort A recipients (both No-BOS and BOS), using CD28% values from the recipients first assays. Numbers in parenthesis at end of the survival curves denote remaining, unafflicted subjects that were censored at 24 months of observation. (B) Actuarial survival curves showing cumulative freedom from major adverse events (retransplantation or death) of CD28% High (n=19) and CD28% Low (n=11) among Cohort B recipients (all of whom had BOS). Tick marks denote interval censored subjects. C.) Actuarial survival curves showing cumulative freedom from major adverse events (retransplantation or death) of CD28% High and CD28% Low among Cohort A recipients with BOS (n=5 in each group).

FIG. 10. Kaplan Meier plot of percent survival of IPF patients over time. Solid line shows patients with percent CD25 greater than 54%, dotted line shows patients with percent CD25 less than or equal to 54%. HR=hazard ratio. CI=confidence interval.

FIG. 11. Kaplain Meier plot of percent survival of IPF patients over time. Solid line shows patients with percent CD4 greater than 31.3%, dotted line shows patients with percent CD4 less than or equal to 31.3%. HR=hazard ratio. CI=confidence interval. p=0.001. HR=4; 95% CI 1.6-9.8.

FIG. 12. Kaplain Meier plot of percent survival of IPF patients over time. Solid line shows patients with percent of CD8 that express CD28 (“CD8+28+”) greater than 21%, dotted line shows patients with percent CD8+28+ less than or equal to 21%. HR=hazard ratio. CI=confidence interval. p=0.005. HR=5.6; 95% CI=1.4-21.7.

FIG. 13. Kaplain Meier plot of percent survival of IPF patients over time. Solid line shows patients with percent of CD4 cells that express CD45RO (“CD4+CD45RO+”) greater than 87.1%, dotted line shows patients with percent CD4+CD45RO+ less than or equal to 87.1%. HR=hazard ratio. CI=confidence interval. p=0.0002. HR=10.4; 95% CI 2.2-50.0.

FIG. 14. Kaplain Meier plot of percent survival of IPF patients over time. Solid line shows patients with percent of CD4 cells that express DR (“CD4DR”) greater than 87.1%, dotted line shows patients with percent CD4DR less than or equal to 87.1%. HR=hazard ratio. CI=confidence interval. p=0.0124. HR=4.9; 95% CI 1.2-19.7.

### Characteristics of CD4 T-Cell Subpopulations in IPF Patients.

FIG. 15A-B. A: The proportions of circulating CD4 T-cells that also expressed CD28 (CD28%) were reduced in many IPF patients. The horizontal line denotes the population means. B: In contrast to autologous CD4+CD28+ cells, the CD4+CD28null T-cells of IPF patients more often express major histocompatibility antigen (MHC) Class II (DR), but less frequently express CD25. CD4+CD28null T-cells of IPF patients less frequently produce transcription factor FoxP3 (a putative marker of regulatory T-cells), but much more frequently produce cytotoxic mediators granzyme B (GB) and perform (Perf). For each measure n=24, and p values for all intergroup comparisons (CD4+CD28+ vrs. CD4+CD28null cells) are <0.0001.

FIG. 16. Cytokine elaborations by autologous CD4 subpopulations of IPF patients. Initial (left) data point in each series represents control unstimulated (basal) condition, while second (right) data point delineates productions of cells after stimulation with plate bound anti-CD3 antibody. These paired specimens (control and stimulated) are also connected by lines. CD4+CD28null cells from IPF patients (open circles with paired specimens connected by dashed lines) tend to elaborate greater amounts of pro-inflammatory and TH1 cytokines (top two rows), whereas CD4+CD28+ cells (open squares with paired specimens connected by solid lines) have an apparent TH2 bias, with the exception of IL-4 production (bottom row) (n=6 randomly-selected specimens in each measure).

FIG. 17. Segregated autologous CD4+CD28+ and CD4+CD28null cells from peripheral blood of IPF patients (n=9) had similar proliferations (determined by BrdU incorporation) in 5-day control (unstimulated) cultures, as well as after stimulation with plate-bound anti-CD3 monoclonal antibody.

FIG. 18A-B. Associations of CD28 expression with pulmonary function. Changes of CD28% with replicate testing (Delta CD28%) were significantly correlated with concomittant interval changes of A.) absolute FVC (Delta FVC) and B.) FVC as a time-dependent rate (Delta FVC/month).

FIG. 19A-B. Associations of CD28% espression with clinical outcome. (A) Survival curves show cumulative freedom from major adverse events (lung transplantation or death) of IPF patients. Those subjects with the most extreme CD28 down-regulation, with CD28 expressed on <82% of their circulating CD4 T-cells (CD28% Low), had much worse outcomes than the cohort with greater proportions of CD4-Tcells that expressed CD28 (CD28% High). Numbers in parenthesis at the ends of survival curves denote remaining, unafflicted subjects that were censored at 12 months of observation. (B) Survival curves showing that cummulative freedom from major adverse events of IPF patients who have either significant CD28 downregulation (CD28% Low) or diffusing capacities for carbon monoxide, as percentages of predicted normal values (DLCO % p) <38, had worse outcomes than the IPF cohort who were both CD28% High and had more normal DLCO % p.

FIG. 20A-H. Localization of CD4, CD3, and granzyme B expressing cells in IPF lungs. (A) and (B) Cells expressing CD4 (Green) and granzyme B (red), respectively, are widely distributed in IPF lung sections. (C) DAPI (blue) stains DNA within cell nuclei. (D) Co-localizations of CD4 and granzyme B (yellow) among nucleated cells are seen in the merged image. Nearly all the intrapulmonary CD4+ cells co-expressed granzyme B. Images (E), (F) and (G) similarly depict CD4 (green), CD3 (red) and DAPI (blue). (H) Merged image shows most of the CD4+ cells also co-expressed CD3. Similar results were present in both IPF lungs. The majority of CD4+ cells in these IPF lung sections co-expressed both granzyme B and CD3 and are, thus, CD4+CD28null T-cells (see also FIG. 15B). All images are 60×.

FIG. 21A-C. Flow cytometry methodology (see also references 20, 52). Aliquots of fresh, live peripheral blood mononuclear cells (PBMNC) were stained with anti-CD 4-allophycocyanin (APC), anti-CD28-fluorescein isothiocyanate (FITC), and phycoerythrin (PE)-conjugated antibodies against other cell epitopes. (A) Ten thousand (10,000) or more live cells were selected for further study, based their side scatter (SSC) and forward scatter (FSC) characteristics (G1). (B) The brightly staining CD4 cells among these also expressed CD3 (Cy-Chrome) and, thus, are T-cells [52]. (C) These CD4 T-cells were further characterized based on their expression of CD28. The proportions of CD4+CD28+ T-cells among the total CD4+ T-cell population (upper left and upper right quadrants) defines the CD28%. The respective proportions of CD4+CD28+ and CD4+CD28null cells that co-expressed other cell determinants of interest (in this case MHC Class II [DR]) were quantitated. Numbers within the delineated region/quadrants denote the proportions of cells with these respective characteristics.

FIG. 22. Repetitively cycled and highly altered CD4 T-cells, identified by their absence of CD28, and denoted with red circles, that were isolated from IPF patients, are relatively resistant to effects of glucocorticoids, compared to “normal” CD4 T-cells (blue squares) isolated from the same patients.

FIG. 23A-H. Expression of (A) CD27; (B) CCR7; (C) CD3; (D) CD80; (E) CTLA-4; (F) ICOS; (G) Itk; and (H) Lck, in CD4+CD28+ versus CD4+CD28null T cells of IPF patients.

### 5. DETAILED DESCRIPTION OF THE INVENTION

For clarity and not by way of limitation, the detailed description of the invention is divided into the following subsections:

(i) biomarkers;

(ii) chronic pulmonary diseases; and

(iii) clinical methods.

### 5.1 Biomarkers

The present invention provides for the use of the following biomarkers:

CD4 protein (see, for example, GenBank Acc. No. AAV38614);

CD28 protein (see, for example, GenBank Acc. No. AAI12086);

CD25 (see, for example, NCBI Ref. Seq. No. NP_{—}000408);

CD8 (see, for example, NCBI Ref. Seqs. NP_{—}001759.3, NP_{—}742100);

CD45RO (see, for example, GenBank Ace. No. AAS46946);

CD27 (see, for example, GenBank Ace. No. AAH12160);

CCR7 (see, for example, NCBI Ref. Seq. No. NP_{—}001829),

CD3 (see, for example, NCBI Ref. Seq. Nos. NP_{—}000064.1; NP_{—}000723.1;

### NP_{—}0010303574.1; NP_{—}000724.1; NP 932170.1);

CD80 (see, for example, GenBank Ace. No. ABK41933.1);

CTLA-4 (see, for example, NCBI Ref. Seq. No. NP_{—}005205.2);

ICOS (see, for example, GenBank Acc. No. AAH28006.1);

Itk (see, for example, GenBank Ace. No. BAA02873.1);

Lck (see, for example, NCBI Ref. Seq. No. NP_{—}005347.3);

HLA-DR;

interleukin 7 receptor (“IL-7R”; see, for example, NCBI Ace. No. AAH20717);

granzyme B (see, for example, NCBI Acc. No. AAA75490); and

perform (see, for example, NCBI Acc. No. AAA60065).

The preceding accession numbers are offered as examples only and are not intended to be limiting.

The biomarkers may be identified by any method known in the art, including, but not limiting to, enzyme linked immunosorbent assay, or Western blot. In addition, more recent technologies, such as those used in the field of proteomics, may be embodied in kits of the invention. Such technologies include the use of microfluidic chips and related technologies as described, for example, in United States Patent Application No. US 2008/0202927; Sorger, 2008, Nature Biotechnol. 26:1345-1346; Li et al., 2002, Mol. Cell. Proteomics 1.2:157; Hou et al., 2006, J. Proteome Res. 5(10):2754-2759; Li et al., 2001, Proteomics 1(8):975-986; Ramsey et al., 2003, Anal. Chem. 75(15):3758-3764; Armenta et al., 2009, Electrophoresis 30(7): 1145-1156; Lynch et al., 2004, Proteomics 4(6):1695-1702; Kingsmore et al., 2003, Curr. Opin. Biotechnol. 14(1):74-81).

As discussed more fully below, in certain cases a particular combination of biomarkers is assessed, for example, the occurrence of CD28 antigen on T-cells bearing CD4. In such cases, it may be desirable to determine levels of CDS28 antigen on T-cells pre-selected as expressing CD4. Accordingly, where a combination of antigens is being assessed, it may be desirable to first collect a population of cells bearing one of the antigens and then testing for the presence of the other. Similarly, if a product of a population of cells (e.g. perforin or granzyme) is being assessed, the population may first be collected and then expression of the product may be determined.

A preferred method of determining antigen expression is flow cytometery. Flow cytometry is a technique for counting, examining, and sorting microscopic particles suspended in a stream of fluid. It allows simultaneous multiparametric analysis of the physical and/or chemical characteristics of single cells flowing through an optical and/or electronic detection apparatus. Fluorescence-activated cell sorting is a specialized type of flow cytometry. It provides a method for sorting a heterogeneous mixture of biological cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest. Fluorescent signals used in flow cytometry, for instance when quantifying and/or sorting cells by any marker present on or in the cell, typically are fluorescently-tagged antibody preparations or fluorescently-tagged ligands for binding to antibodies or other antigen-, epitope- or ligand-specific agent, such as with biotin/avidin binding systems or fluorescently-labeled and optionally addressable beads (e.g. LUMINEX® microspheres). Virtually any marker or combination of markers can be detected by the optics and/or electronics of a flow cytometer can be measured or exploited in flow cytometer methods, including; without limitation, cell surface markers, intracellular and nuclear antigens, DNA, RNA, cell pigments, cell metabolites, protein modifications, transgenic proteins, enzymatic activity, apoptosis, cell viability, cell oxidative state, etc.

In one example of flow cytometry describe herein, fluorochrome-labeled monoclonal antibodies with specific avidity against the markers of interest to the cells (under specific conditions). Then you run those cells thru the flow cytometer and it counts events (e.g., the total number of cells, number of these with the fluorochrome marker, etc.). Antibodies directed to the various biomarkers are commercially available. One vendor that provides antibodies to at least some of the biomarkers is BD Pharmingen.

In specific, non-limiting embodiments, the present invention provides for a kit comprising a means for detecting at least one of the above biomarkers. Means for detecting a biomarker are known in the art, and include, for example, the use of a capture agent, which optionally is detectably labeled, where the capture agent may be used together with a detection agent that binds to the biomarker and/or the capture agent. A capture agent may be, for example and not by limitation, an antibody, a portion of an antibody such as a Fab or Fab2 fragment, a single chain antibody, a receptor for the biomarker or a portion thereof or a ligand for the biomarker or a portion thereof. Likewise, a detection agent may be, for example and not by limitation, an antibody, a portion of an antibody such as a Fab or Fab2 fragment, a single chain antibody, a receptor for the biomarker or capture agent or a portion thereof or a ligand for the biomarker or capture agent or a portion thereof. The capture agent and/or detection agent may be detectably labeled using a radioactive label, a fluorescent label, a chemical label, an oligonucleotide label, an enzymatic label, or a protein label (e.g. a fluorescent protein such as Green Fluorescent Protein). In specific, non-limiting embodiments, said kit may comprise antibody or other capture agent directed to one or more of, or two or more of, or three or more of, or four or more of, CD4, CD28, CD25, CD8, HLA DR, CD45Ro, CD27, CCR7, granzyme B, FoxP3, IL-7R, or perform, optionally together with a detection agent, and optionally with a package insert describing one or more clinical method set forth below. In such kits, in non-limiting embodiments, the number of different biomarkers that may be detected by capture agents in the kit may be less than 100 or less than 50 or less than 25 or less than 10 or less than 5. In specific non-limiting embodiments, said kit may comprise antibody or other capture agent directed to CD4, CD28 and CD25.

### 5.2 Chronic Pulmonary Diseases

Chronic pulmonary diseases to which the present invention may be applied include, but are no limited to, chronic rejection following lung transplant, IPF, chronic obstructive pulmonary disease, cystic fibrosis, pulmonary hypertension, inflammatory lung disease associated with an autoimmune disease, and lung disease associated with sarcoidosis and scleroderma.

### 5.3 Clinical Methods

In various non-limiting embodiments, the present invention provides for a method of determining the risk that a subject suffering from a chronic pulmonary disease will suffer a severe adverse event, comprising determining whether, in a sample comprising T-cells collected from the subject, one or more of two or more of, three or more of, four or more of, five or more of, six or more of, seven or more of, eight or more of, nine or more of ten or more of, eleven or more of, twelve or more of, etc. the following is present:

the proportion of CD4+CD28null cells among the Cd4+ T-cell population is elevated, where said elevation indicates that the subject is at increased risk of suffering a severe adverse event, and in specific non-limiting embodiments if the percent of CD4+CD28+ among CD4+ cells is less than about 90 percent, the subject is at increased risk of suffering a severe adverse event;

the proportion of CD4+ cells among the circulating peripheral blood mononuclear cell (PBMNC) population is decreased, where said decrease indicates that the subject is at increased risk of suffering a severe adverse event, and in specific non-limiting embodiments if the percent of CD4+ cells in the PBMNC population is less or equal to about 31.1 percent, the subject is at an increased risk of suffering a severe adverse event;

the proportion of CD25+ cells among the T-cell population is decreased, where said decrease indicates that the subject is at increased risk of suffering a severe adverse event, and in specific non-limiting embodiments if the percent of CD25+ cells in the T cell population is less than or equal to about 54 percent, then subject is at an increased risk of suffering a severe adverse event;

the proportion of CD28+ cells among the CD8+ T-cell population is decreased, where said decrease indicates that the subject is at increased risk of suffering a severe adverse event, and in specific non-limiting embodiments if the percent of CD8+CD28+ cells in the CD8+ T cell population is less than or equal to about 21 percent, then subject is at an increased risk of suffering a severe adverse event; the proportion of CD4+CD45RO+ cells among the CD4+ T-cell population is decreased, where said decrease indicates that the subject is at increased risk of suffering a severe adverse event, and in specific non-limiting embodiments if the percent of CD45RO+ cells in the CD4+ T cell population is less than or equal to about 54 percent, then subject is at an increased risk of suffering a severe adverse event;

the proportion of CD4+HLA−DR+ cells among the CD4+ T-cell population is increased, where said increase indicates that the subject is at increased risk of suffering a severe adverse event, and in specific non-limiting embodiments if the percent of CD4+HLA-DR+ cells in the CD4+ T cell population is greater than about 87.1 percent, then subject is at an increased risk of suffering a severe adverse event;

the proportion of CD27+ cells among the CD4+ T-cell population is decreased relative to a healthy control, where said decrease indicates that the subject is at increased risk of suffering an adverse event;

the proportion of CCR7+ cells among the CD4+ T-cell population is decreased relative to a healthy control, where said decrease indicates that the subject is at increased risk of suffering an adverse event, and in specific non-limiting embodiments the proportion of CD4+CD28null T-cells from the subject expressing CCR7 is less than 50 percent of the proportion of CD4+CD28+ T-cells from the subject that express CCR7;

the proportion of IL-7R+ cells among the CD4+ T-cell population is decreased relative to a healthy control, where said decrease indicates the subject is at increased risk of suffering an adverse event;

the proportion of CD3+ cells among the CD4+ T-cell population is decreased relative to a healthy control, where said decrease indicates the subject is at increased risk of suffering an adverse event, and in specific non-limiting embodiments the proportion of CD4+CD28null T-cells from the subject expressing CD3 is less than 50 percent of the proportion of CD4+CD28+ T-cells from the subject that express CD3;

the proportion of CD80+ cells among the CD4+ T-cell population is increased relative to a healthy control, where said decrease indicates the subject is at increased risk of suffering an adverse event, and in specific non-limiting embodiments the proportion of CD4+CD28null T-cells from the subject expressing CD80 is more than 150 percent of the proportion of CD4+CD28+ T-cells from the subject that express CD80;

the proportion of CTLA-4+ cells among the CD4+ T-cell population is decreased relative to a healthy control, where said decrease indicates the subject is at increased risk of suffering an adverse event, and in specific non-limiting embodiments the proportion of CD4+CD28+ null T-cells from the subject expressing CTLA4 is less than 50 percent of the proportion of CD4+CD28+ T-cells from the subject that express CTLA-4;

the proportion of ICOS+ cells among the CD4+ T-cell population is decreased relative to a healthy control, where said decrease indicates the subject is at increased risk of suffering an adverse event, and in specific non-limiting embodiments the proportion of CD4+CD28null T-cells from the subject expressing ICOS is less than 30 percent of the proportion of CD4+CD28+ T-cells from the subject that express ICOS;

the proportion of Itk+ cells among the CD4+ T-cell population is decreased relative to a healthy control, where said decrease indicates the subject is at increased risk of suffering an adverse event, and in specific non-limiting embodiments the proportion of CD4+CD28null T-cells from the subject expressing Itk is less than 50 percent of the proportion of CD4+CD28+ T-cells from the subject that express ITk;

the proportion of Ltk+ cells among the CD4+ T-cell population is decreased relative to a healthy control, where said decrease indicates the subject is at increased risk of suffering an adverse event, and in specific non-limiting embodiments the proportion of CD4+CD28null T-cells from the subject expressing Ltk is less than 50 percent of the proportion of CD4+CD28+ T-cells from the subject that express Ltk;

the amount of granzyme B produced by CD4+ T-cells of the subject is increased relative to T-cells of a healthy control, where said increase indicates that the subject is at increased risk of suffering an adverse event

the amount of FoxP3 produced by CD4+ T cells of the subject is decreased relative to T cells of a healthy control, where said decrease indicates that the subject is at increased risk of suffering an adverse event; and

the amount of perform produced by CD4+ T-cells of the subject is increased relative to T-cells of a healthy control, where said increase indicates that the subject is at increased risk of suffering an adverse event; where the adverse event is selected from the group consisting of death within one year, lung transplant desirable or deemed necessary within one year, (in transplant patients) incidence of BOS, resistance to therapy with cyclosporine or other immunosuppressive agent, decrease in DLCO, or decrease in FVC, or decrease in FEV1.

The foregoing methods may be comprised in a broader method of treating a subject, wherein if the subject is found to be at increased risk of a severe adverse event, there is a further step of recommending an interventional step, for example, but not limited to, lung transplant, lung biopsy (transplant recipient only), closer observation and serial PFT testing, augmentation or addition or substitution of immunosuppressive medications

### 6. EXAMPLE

### CD28 Down-Regulation of CD4 Cells as a Marker for Graft Dysfunction in Lung Transplant Recipients

In the experiments described below, the proportion of circulating CD4 T cells that lacked expression of CD28 was determined by flow cytometry in individual lung transplant recipients and correlated with the subjects' diagnoses, pulmonary function tests (PFTs), and clinical outcome. In addition, expression of cell activation markers, intracellular FoxP3, cytotoxic mediator and cytokine production, and resistance to cyclosporine of the CD4+CD28null cells from recipients were compared to those of autologous, and more typical, CD4 T cells that express CD28, through flow cytometry and functional assays. Some of the results of these studies have been previously reported in the form of abstracts (Studer S M, et al. Altered T-cell phenotypes in lung transplant recipients with obliterative bronchiolitis [abstract]. Am J Respir Crit. Care Med 2004; 169:A69; and Studer S M, et al. Unusual CD4+CD28 null cells in lung transplant recipients with BOS [abstract]. Proc Am Thorac Soc 2005; 2:A859).

### 6.1 Materials and Methods

Subjects.

Lung allograft recipients were recruited during routine outpatient evaluations. Two successive Cohorts of transplant patients were examined: Cohort A and Cohort B. The initial subject population (Cohort A) were recruited during the first two years of this study and were consecutive recipients seen in clinic who fulfilled inclusion criteria. Based on the findings of these interval analyses, and in order to more productively characterize CD4+CD28null cells per se, which are predominantly found in recipients with bronchiolitis obliterans syndrome (“BOS”; see below), subsequent recruitments (Cohort B), consisted of recipients with preexisting diagnoses of BOS (again, otherwise fulfilling the inclusion criteria). Aside from inclusion of only BOS subjects at this time, these Cohort B recruitments were patients that were sequentially evaluated and consented in outpatient clinics. Any recipient, during enrollment of either Cohort, was excluded from study if they had malignancies, evidence of current or recent active infections (within the preceding four weeks), allografts other than their initial lung transplantation, known ACR at the time of their CD4 assay, profound anemia (Hct <28%), or increases of immunosuppression within the preceding four weeks. Some recipients consented to replicate assays, but, unless noted otherwise, only results from first determinations were analyzed here. Routine clinical protocols are also described elsewhere (Husain S, et al. Voriconazole prophylaxis in lung transplant recipients. Am J Transplant 2006; 6:3006-16). Healthy normal volunteers were recruited by solicitation, concurrent with studies of Cohort A recipients. Written informed consent was obtained from all subjects, per the Institutional Review Board (IRB).

Diagnoses of BOS were established by physiologic and clinical consensus criteria (Estenne M, et al. Bronchiolitis obliterans syndrome 2001: an update of the diagnostic criteria. J Heart Lung Transplant 2002; 21:297-310). According to that reference, and without limitation, BOS can be defined as a decrease in FEV1 of >20% determined by the average of 2 measurements made at least 3 weeks apart, without patient use of an inhaled bronchodilator. Patients having a single measurement of decreased FEV1 are not evaluated until a second measurement is obtained at least 3 weeks after the initial data point. Because BOS is meant to represent a persistent alteration in lung function, additional values of FEV1, which may be obtained during this 3-week period, should also show a significant decrease from baseline value. The date at which a patient enters the new BOS stage is the date of the first of the 2 measurements used to confirm the stage. In case of a concomitant decrease in vital capacity (VC) and FEV1, a restrictive ventilatory defect should be excluded before categorizing the patient in a new BOS stage. Also, recipients should be free of any number of multiple confounding conditions, like pneumonia, pneumothorax or pleural effusions, neuromuscular weakness, etc. that can confound pulmonary function tests. Cytomegalovirus (CMV) or other viral infections at any time after transplantation were determined by positive viral cultures from bronchoalveolar or other specimens, diagnostic histopathology, the presence of pp 65 antigenemia, or, more recently, by quantitative polymerase chain reaction.

Induction Immunosuppression Regimens.

Protocols for immunosuppression induction have been described previously (Husain 5, et al. Am J Transplant 2006; 6:3006-3016; Keenan R J, et al. Clinical trial of tacrolimus versus cyclosporine in lung transplantation. Ann Thorac Surg 1995; 60:580-585; McCurry K R, et al. Early outcomes in human lung transplantation with Thymoglobulin or Campath-1H for recipient pretreatment followed by posttransplant tacrolimus near-monotherapy. J Thorac Cardiovasc Surg 2005; 130:528-37). Subjects who had transplantations either received: an intravenous (i.v.) dose of methylprednisolone (500 mg) and azathioprine (4 mg/kg) immediately prior to revascularization of the allograft (Keenan R J, et al. Ann Thorac Surg 1995; 60:580-585); daclizumab on days 0, 14, 28, 42 and 56, post transplantation; preoperative induction with rabbit antithymocyte globulin (Thymoglobulin) (McCurry K R, et al. J Thorac Cardiovasc Surg 2005; 130:528-37); or alemtuzumab 30 mg (see Tables 2-4).

Maintenance Immunosuppression.

Recipients were maintained on tacrolimus-based immunosuppression with doses adjusted to attain trough concentrations of 15-20 ng/ml for 12 months after transplantation, and 12-15 ng/ml thereafter, along with azathioprine, and prednisone (from 5 to 7.5 mg daily), with or without mycophenolate mofetil.

Allograft Rejection Diagnoses and Treatments.

Acute cellular rejection (ACR) episodes were diagnosed by histologic assessments of lung biopsies (Yousem S A, et al. Revision of the 1990 working formulation for the classification of pulmonary allograft rejection: Lung Rejection Study Group. J Heart Lung Transplant 1996; 15:1-15). Diagnoses of bronchiolitis obliterans syndrome (BOS) were established by strict adherence to clinical consensus criteria (Estenne M, et al. J Heart Lung Transplant 2002; 21:297-310). As an example, diagnoses of BOS were never initially established in study subjects who also had coexistent comorbidities, including acute allograft rejection or infections (Estenne M, et al. J Heart Lung Transplant 2002; 21:297-310). ACR episodes were treated throughout the study period with an oral prednisone taper (mild, initial ACR) or i.v. methylprednisolone 1 gm daily for 3 days (moderate-severe or persistent mild ACR). Rabbit antithymocyte globulin (RATG) was used for corticosteroid resistant rejection. Initial and progressive BOS was treated with methylprednisolone and RATG, respectively. Mycophenolate mofetil and sirolimus were substituted for azathioprine and tacrolimus, respectively, in selected cases with recurrent acute rejection and/or progressive BOS.

Antimicrobial Prophylaxis.

Details of antimicrobial prophylaxis regimens are described elsewhere (Husain S, et al. Prospective assessment of Platelia Aspergillus galactomannan antigen for the diagnosis of invasiveaspergillosis in lung transplant recipients. Am J Transplant 2004; 4:796-802). In brief, cytomegalovirus (CMV) infection at any time following transplantation was determined by positive viral cultures from bronchoalveolar or other specimens, diagnostic histopathology, or the presence of pp 65 antigenemia using the following criteria: 1) among recipients who were CMV IgG negative prior to their transplantations, any subsequent pp 65 antigenemia was considered a positive finding; and 2) among other recipients, post-transplant pp 65 antigenemia >10/200,000 counted cells was considered positive. Later, a quantitative CMV PCR assay replaced the pp 65 antigen determination as the clinical monitoring test for CMV infections. Any positive PCR was considered significant in recipients who were previously CMV IgG negative, and >100 viral copies was considered a positive result among those with pre-existing CMV IgG. Ganciclovir was employed as CMV prophylaxis in the early post-operative period, as reported previously (Husain S, et al. Am J Transplant 2004; 4:796-802), and initially preemptive therapy with this agent for CMV pp 65 antigenemia was employed. Later, all recipients received valganciclovir 450 mg twice daily (with dosage adjusted for renal dysfunction) as CMV prophylaxis. Valganciclovir was continued for 6 months in CMV seronegative recipients of seropositive allografts, and for 3 months in all others. Initially, recipients without known fungal colonization received antifungal prophylaxis with fluconazole 200 mg orally daily for 3 months. Oral itraconazole 200 mg twice daily was used instead among those with pre- or post transplant colonization with Aspergillus spp. (except A. niger), with or without inhalational amphotericin B deoxycholate. Later, oral voriconazole 200 mg twice daily was employed for 4 months as antifungal prophylaxis.

Assignments of Diagnoses and Treatment Decisions.

Diagnoses for the study subjects were assigned after review of all clinical data by investigators blinded to experimental T-cell assay results. Decisions regarding treatments (including retransplantation) were made by clinicians completely unaware of T-cell assay results. Conversely, the experimental laboratory assays (see below) were performed and analyzed by investigators blinded to details of subject identities, demographics, treatments, or clinical courses.

Cell Preparations and Flow Cytometry.

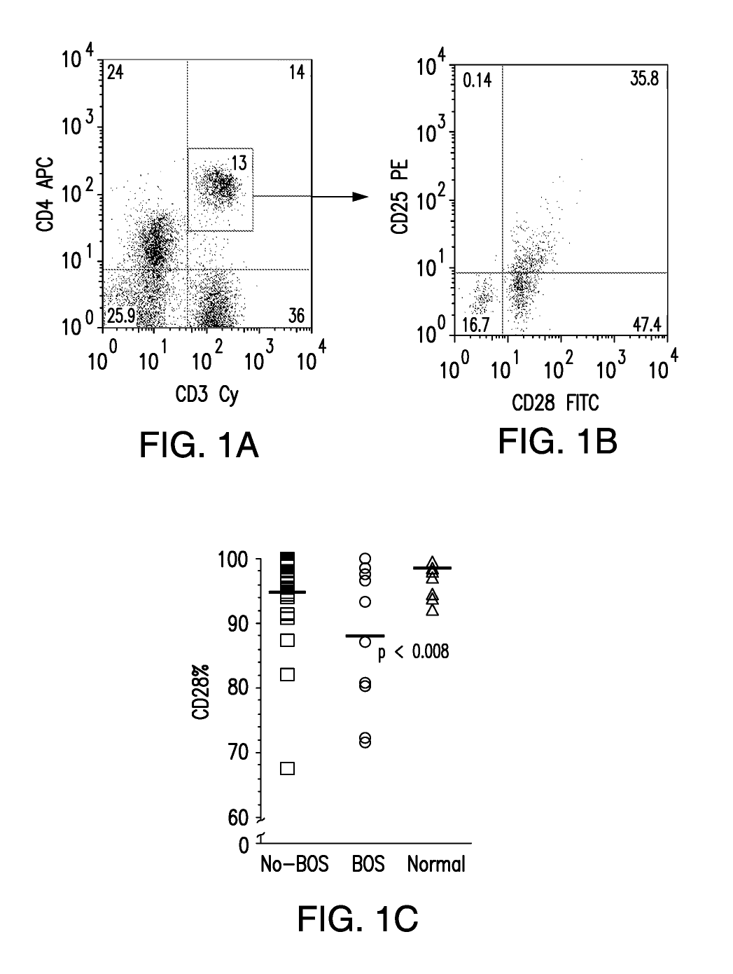

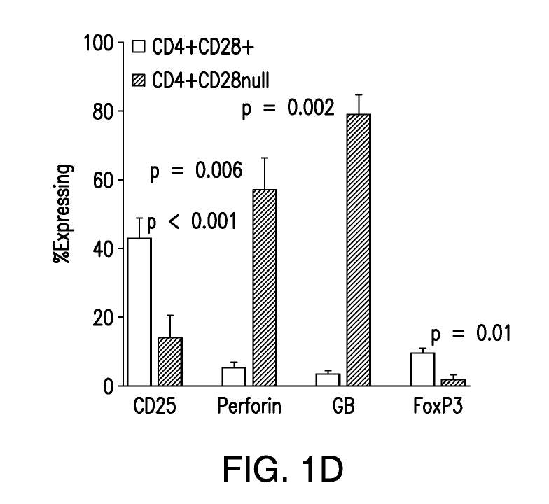

Peripheral blood mononuclear cells (PBMNC) were isolated by density gradient centrifugation from peripheral blood for use in flow cytometry or cell assays. Phenotypic characterizations were ascertained using fluorochrome-conjugated monoclonal antibodies, including isotype control antibodies that were purchased, unless otherwise denoted, from BD Pharmingen (San Diego, Calif.). Flow cytometry quantitations were performed on >10,000 live cells and analyzed using a BD FACSAria™ or BD FACSCalibur™ (BD Bioscience, San Jose, Calif.). To exclude confounding by CD4dim monocytes, CD4bright (CD4 T cells) were selectively gated. CD28 and other phenotypic markers were quantitated within respective CD4+CD28+ and CD4+CD28null subpopulations (FIGS. 1A-1B). Intracellular mediators (perforin, granzyme B, FoxP3) were quantitated among fresh PBMNC by staining with monoclonal antibodies for CD4 and CD28, and then fixing and permeabilizing cells prior to incubation with antibodies having specificities for these intracellular molecules (anti-FoxP3 antibodies were purchased from eBioscience, San Diego, Calif.). The respective CD4+CD28+ and CD4+CD28null subpopulations were then individually gated, and the frequency of intracellular staining in each CD4 T-cell subpopulation was quantitated using flow cytometry. The specificities of antibodies used here were confirmed by comparisons to isotype controls. CD4 T-cells cells for functional assays were isolated from PBMNC by negative selection with immunomagnetic beads (Miltenyi Biotech, Auburn, Calif.). CD28null cells and CD28+ cells were then individually segregated from the CD4 T-cells by staining with azide-free anti-CD28-phycoerythrin, followed by positive selection of the latter with anti-phycoerythrin immunomagnetic beads (Miltenyi Biotech). Purity of the respective cell populations was 95% or greater by flow cytometry.

Cytokine Assays.

A total of 1×10⁵ CD4 T cells (autologous CD28+ and CD28null) were separately cultured in 96-well plates with 200 μL of complete RPMI media in 7% CO2 in both stimulated (10 μg/ml plate-bound anti-CD3 antibody [OKT3]) and unstimulated (basal) conditions. Culture media was removed after two days, centrifuged, and supernatants were analyzed for cytokine productions using a protein suspended bead array platform (Bio-Plex™) multiplex kit (Bio-Rad, Hercules, Calif.), following the manufacturer's protocols. Cytokine levels within concurrent media controls were subtracted from specimen values.

Inhibition of Proliferation by Cyclosporine A (CsA)

Segregated CD28+ and CD28null CD4 T-cell subpopulations were individually cultured in duplicate wells coated with anti-CD3 monoclonal antibody (OKT3) (see above). Autologous nurse cells, positively selected during the initial CD4 T-cell isolations (see above) were added in a 3:1 nurse cell: CD4 T-cell ratio. In order to eliminate potential confounding by CD4dim monocytes, all CD4+ cells were depleted from the nurse cells (<<1% residual), prior to their use in cultures, by positive selection with anti-CD4-coated magnetic beads (Invitrogen, Carlsbad, Calif.). Cyclosporine (Novartis, East Hanover, N.J.) to a final concentration of 100 ng/ml was added to half the wells. Proliferation within the respective CD4 subpopulations were measured by bromodeoxyuridine (BrdU) incorporation, using reagents and methods supplied in a kit (BD Pharmingen). In brief, BrdU in a final concentration of 10 uM was added to cultures two days prior to their harvests. After 5±1 days of culture, cells were harvested from wells and stained with anti-CD4, fixed and permeabilized, and then incubated with anti-BrdU and 7-amino-actinomycin (7-AAD). The proportion of proliferating CD4+ T cells (BrdU+) among the viable cells (≧diploid DNA content) was determined by flow cytometry.

Statistical Analyses.

Intergroup comparisons of continuous or ordered data were analyzed by Mann-Whitney or Kruskal-Wallis for two and three group comparisons, respectively. Dichotomous variables were analyzed by Chi-square. Correlations between continuous variables were established by Spearman rank order. Survival analyses were performed using product-limit estimation, with comparisons by Log rank (Mantel-Cox). The relationships of dichotomous variables (e.g., CMV, BOS) with CD28 expression were analyzed by bivariate logistic regression. Data were analyzed using StatView™ (SAS Institute, Cary, N.C.). Receiver operator characteristic (ROC) curves were generated using Intercooled Stata 9.0 (Stata Corp., College Station, Tex.). Significance was defined as p<0.05. Data are depicted as mean±SEM.

Results

CD28 Studies. FIGS. 1A-E show characteristics of CD4 T-cell subpopulations in lung allograft recipients. In FIGS. 1A-B, these characteristics were determined by flow cytometry methodology. In FIG. 1A, T cells that stained brightly with anti-CD4 monoclonal antibody conjugated to allophycocyanin and CD3 conjugated to Cy-Chrome were gated for subsequent determinations of cellular expressions. In FIG. 1B, expressions of other phenotypic markers, in this example denoted by anti-CD25 phycoerythrin (PE) antibody staining, were individually determined in autologous CD4+CD28+ and CD4+CD28null subpopulations. In the example here, these respective subpopulation are denoted by the presence or absence, respectively, of costaining with anti-CD 28 antibody conjugated to fluorescein isothiocyanate (FITC). In FIG. 1C, the percentages of circulating CD4 T cells that also expressed CD28 (CD28%) were reduced in lung transplant recipients with BOS in comparisons with healthy normal (nontransplanted) control subjects. Horizontal lines denote population means. The No-BOS recipient with a CD28% of 67.4 had obliterative bronchiolitis on lung biopsy, but normal expiratory flow at the time of this CD4 assay. In contrast to autologous CD4+CD28+, FIG. 1D shows that the CD4+CD28null T cells from lung transplant recipients with BOS less often express activation marker CD25 (n=16). Assays in later Cohorts of consecutive, randomly selected subjects also show that the CD4+CD28null cells from recipients with BOS much more frequently produce the cytoxic mediators, perform and granzyme B (both n=10), but less often express FoxP3+(n=6), in comparisons to autologous CD4+CD28+ cells.

CD28 Down-Regulation on CD4 T Cells Is Associated with BOS.

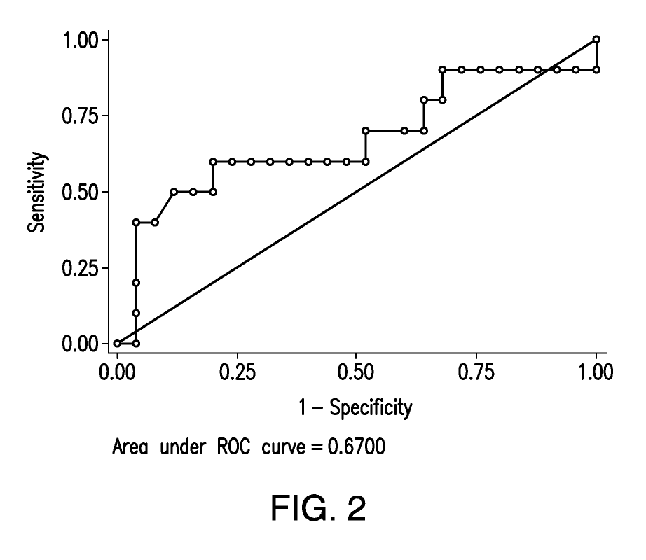

The proportion of circulating CD4 T cells that expressed CD28 (CD28%) were determined among 35 consecutive recipients, seen in routine clinic visits, who fulfilled inclusion criteria (Cohort A) (as described above in the Methods section, above). Laboratory investigators were blinded to the identity and characteristics of these subjects. The CD28% among the 25 lung transplant recipients with no evidence of BOS (No-BOS) (Estenne M, et al. J Heart Lung Transplant 2002; 21:297-310) was 94.6+1.5, and similar to that of 16 healthy, normal volunteers (98.2+6 0.5). Three of these recipients (including one with a CD28% of 67.4) had prior lung biopsies showing obliterative bronchiolitis (OB) (Yousem S A, et al. J Heart Lung Transplant 1996; 15:1-15), but had normal expiratory flow at the time of their CD4 assays, and are classified here as No-BOS. All three subsequently had deteriorations of expiratory flow and formally met BOS criteria (Estenne M, et al. J Heart Lung Transplant 2002; 21:297-310) between 2 and 10 months later. Ten (10) of the randomly recruited lung transplant recipients in Cohort A had established diagnoses of BOS prior to these CD4 studies (unbeknownst to the laboratory investigators). CD28% values among these BOS recipients were comparatively diminished (FIG. 1C). Cutoff values for CD28% of 0.9 seemed to provide the best compromise of specificity (the higher consideration) and sensitivity for concurrent BOS, as confirmed by receiver operator characteristic curve analysis (see FIG. 2 and Table 1, which provides the detailed report for FIG. 2). FIG. 2 shows the nonparametric Receiver Operative Characteristics (ROC) curve for CD4+CD28+/total CD4 (CD28%) determinations, as a clinical predictive test for BOS among the initial subject population (Cohort A). The ROC is plotted between the true positive rate (sensitivity) on the y-axis, and the false positive rate (1-specificity) on the x-axis. Area under curve (AUC) represents the accuracy of the CD28% and was 0.67 (standard error 0.12, 95% C.I. 0.44-0.90),

TABLE 1
Detailed Report of Sensitivity and Specificity for FIG. 2

| Cutpoint | Sensitivity | Specificity | Classified | LR+ | LR− |
|---|---|---|---|---|---|
| (>=0) | 100.00% | 0.00% | 28.57% | 1.0000 | |
| (>=.1) | 90.00% | 0.00% | 25.71% | 0.9000 | |
| (>=.3) | 90.00% | 4.00% | 28.57% | 0.9375 | 2.5000 |
| (>=.4) | 90.00% | 8.00% | 31.43% | 0.9783 | 1.2500 |
| (>=.7) | 90.00% | 12.00% | 34.29% | 1.0227 | 0.8333 |
| (>=.8) | 90.00% | 16.00% | 37.14% | 1.0714 | 0.6250 |
| (>=1) | 90.00% | 20.00% | 40.00% | 1.1250 | 0.5000 |
| (>=1.1) | 90.00% | 24.00% | 42.86% | 1.1842 | 0.4167 |
| (>=1.3) | 90.00% | 28.00% | 45.71% | 1.2500 | 0.3571 |
| (>=1.5) | 90.00% | 32.00% | 48.57% | 1.3235 | 0.3125 |
| (>=2) | 80.00% | 32.00% | 45.71% | 1.1765 | 0.6250 |
| (>=2.3) | 80.00% | 36.00% | 48.57% | 1.2500 | 0.5556 |
| (>=2.6) | 70.00% | 36.00% | 45.71% | 1.0938 | 0.8333 |
| (>=3) | 70.00% | 40.00% | 48.57% | 1.1667 | 0.7500 |
| (>=3.2) | 70.00% | 48.00% | 54.29% | 1.3462 | 0.6250 |
| (>=3.3) | 60.00% | 48.00% | 51.43% | 1.1538 | 0.8333 |
| (>=3.7) | 60.00% | 52.00% | 54.29% | 1.2500 | 0.7692 |
| (>=4) | 60.00% | 56.00% | 57.14% | 1.3636 | 0.7143 |
| (>=4.5) | 60.00% | 60.00% | 60.00% | 1.5000 | 0.6667 |
| (>=4.7) | 60.00% | 64.00% | 62.86% | 1.6667 | 0.6250 |
| (>=5.4) | 60.00% | 68.00% | 65.71% | 1.8750 | 0.5882 |
| (>=5.7) | 60.00% | 72.00% | 68.57% | 2.1429 | 0.5556 |
| (>=6.1) | 60.00% | 76.00% | 71.43% | 2.5000 | 0.5263 |
| (>=6.8) | 60.00% | 80.00% | 74.29% | 3.0000 | 0.5000 |
| (>=8.6) | 50.00% | 80.00% | 71.43% | 2.5000 | 0.6250 |
| (>=9.3) | 50.00% | 84.00% | 74.29% | 3.1250 | 0.5952 |
| (>=12.7) | 50.00% | 88.00% | 77.14% | 4.1667 | 0.5682 |
| (>=18.1) | 40.00% | 92.00% | 77.14% | 5.0000 | 0.6522 |
| (>=19.5) | 40.00% | 96.00% | 80.00% | 10.0000 | 0.6250 |
| (>=27.7) | 20.00% | 96.00% | 74.29% | 5.0000 | 0.8333 |
| (>=28.4) | 10.00% | 96.00% | 71.43% | 2.5000 | 0.9375 |
| (>=32.6) | 0.00% | 96.00% | 68.57% | 0.0000 | 1.0417 |

| | | | | |
|---|---|---|---|---|
| (>32.6) | 0.00% | 100.00% | 71.43% | 1.0000 |

A total of 5 (50%) of the 10 recipients with BOS had values for CD28% less than 90, whereas only 3 (12%) of the 25 No-BOS subjects had CD28% values less than 90 (TABLE 2).

TABLE 2
Characteristics of Cohort A: random,
consecutive, recipient recruitments

| | (CD4⁺CD28% ≧ 90) | (CD4⁺CD28% > 90) |
|---|---|---|
| | CD28% High | CD28% Low |
| n | 27 | 8 |
| Age (years) | 50.3 ± 1.9 | 51.4 ± 2.8 |
| Recipients with BOS | 5 (19) | 5 (63)* |
| at time of T-cell | | |
| assays, n (%) | | |
| Male, n (%) | 12 (44) | 5 (63) |
| Months since Transplant | 39 ± 9 | 53 ± 13 |
| CD28% | 96.8 ± 0.5 | 78.6 ± 2.6† |
| Transplantation | | |
| Type -n (%) | | |
| SL | 13 (48) | 4 (50) |
| DL | 12 (44) | 4 (50) |
| HL | 2 (7) | 0 (0) |
| Pretransplant | | |
| Diagnoses -n (%) | | |
| COPD | 10 (37) | 6 (75) |
| Pulmonary Fibrosis | 8 (30) | 1 (13) |
| Cystic Fibrosis | 3 (11) | 0 |
| PA Hypertension | 3 (11) | 0 |
| Sarcoidosis/Scleroderma | 3 (11) | 1 (13) |
| Prior CMV -n (%) | 7 (26) | 4 (50) |
| Interval Since | 23 ± 7 | 16 ± 9 |
| CMV (months) | | |
| ACR-n (%) | 8 (30) | 2 (25) |
| ACR Grade | 1.8 ± 0.4 | 2.0 ± 0.0 |
| Induction Regimens - (%) | | |
| Glucocorticosteroid/ | 14 (51) | 7 (88) |
| Azathioprine | | |
| Daclizumab | 4 (15) | 0 |
| RATG | 6 (22) | 1 (13) |
| Alemtuzumab | 3 (11) | 0 |
| Donor:Recipient MHC | | |
| Mismatches | | |
| Class I | 3.5 ± 0.2 | 3.9 ± 0.4 |
| Class 11 | 3.0 ± 0.2 | 2.7 ± 0.4 |

In Table 2, the following abbreviations are used: ACR=acute cellular allograft rejection; BOS=bronchiolitis obliterans syndrome; CMV=cytomegalovirus; COPD=chronic obstructive pulmonary disease; DL=double-lung transplantation; HL=heart/lung transplantation; MHC=major histocompatibility complex; PA=pulmonary artery; RATG=rabbit antithymocyte globulin; SL=single-lung transplantation. “CD4+CD28%≧90 (CD28% High)” denotes those recipients in whom 90% or more of their circulating CD4 T cells also expressed CD28, whereas recipients in whom CD28 was expressed on fewer than 90% of their circulating CD4 T cells are denoted as “CD4+CD28%<90 (CD28% Low)”. “Previous CMV” describes subjects who had CMV infections prior to these CD4 T-cell assays, where “Interval since CMV” describes the time interval between these infections and the CD4 assays. For ACR, grade was established by histologic criteria (Yousem S A, et al. J Heart Lung Transplant 1996; 15:1-15). One of these subjects (a CD28% High subject) was taking rapamycin at the time of their CD4 assay. The asterisk (*) denotes P<0.016 (χ²), and the dagger (†) denotes P<0.0001 (Mann-Whitney). None of the other comparisons were statistically significant.

Aside from BOS prevalence, there were no apparent intergroup differences of demographic or other clinical parameters that could easily account for these CD28 variances (Table 2). CD4+CD28null cells can reportedly accumulate with aging, but this typically occurs in subjects who are much older than these recipients (Vallejo A N, et al. Trends Mol Med 2004; 10:119-124; Vallejo A N. Immunol Rev 2005; 205:158-169). Furthermore, both subject groups had equivalent ages (Table 2), and there was also no correlation between age and extent of CD28 expression in these subjects (rs=0.04; P=0.8). In addition, CD28 down-regulation was also not seen in our healthy (nontransplanted) control subjects, who were carefully screened to exclude subjects with underlying immunologic and other “normal” disorders of senescence, despite their greater ages (58.3±2.6 yr).

CD28 down-regulation (albeit to an apparently lesser degree) has also been described among renal transplant recipients after CMV infections (van Leeuwen E M M, et al. J Immunol 2004; 173:1834-41), but we could not discern a confounding effect of these infections here. The proportion of previously infected recipients tended to be greater in the CD28% Low group, although the intergroup difference in prevalence of prior CMV infections (Table 2) was not statistically significant by Chi-square test (P=0.39). Similarly, bivariate logistic regression analyses confirmed a significant independent association between CD28% Low and BOS (odds ratio=6.4; 95% confidence interval=1.1-37.4; P=0.04), whereas CD28 expression did not seem to associate with prior CMV (odds ratio=2.0; confidence interval=0.3-11.8; P=0.46). Furthermore, of the 11 recipients with prior CMV, 7 (64%) were CD28% High, and half of the CD28% Low recipients had no history of these viral infections. No subject here had CMV diagnosed at the time of their CD4 assays or for at least 2 months thereafter.

Because acute cellular allograft rejection (ACR) is nearly ubiquitous among lung allograft recipients within the first year after transplantation (Trulock E P, et al. J Heart Lung Transplant 2006; 25:880-892, Yousem S A, et al. J Heart Lung Transplant 1996; 15:1-15), potential confounding by this complication could also diminish the significance of CD28 down-regulation as an indicator of CR. Every lung transplant subject studied here had one or more ACR episodes prior to their CD28 measurements. In addition, a small number of subjects unexpectedly had acute rejection on lung biopsies that were performed concurrently with their T-cell assays, and these events occurred equally frequently in both groups (Table 2). For the most part, these incidental (and cryptic) cases were relatively mild (grade I-II) (Yousem S A, et al. J Heart Lung Transplant 1996; 15:1-15). However, three recipients had unsuspected grade III acute rejection at the time of their CD4 assays, and all of these were in the CD28% High group. CD28 down-regulation also does not appear to be a simple function of cellular accumulation over time since transplantation, given the considerable overlap of elapsed intervals among the two populations (Table 2) and the absence of a meaningful correlation between time since transplantation and CD28% (rs=−0.18; P=0.3).

Similarly, there were no simple differences among the type of transplantation, induction regimens, or pretransplant diagnoses that could account for the differences of CD28 expression (Table 2). There was a relative excess of subjects with pretransplant diagnoses of chronic obstructive pulmonary disease, and fewer proportions of patients with pulmonary fibrosis among the CD28% Low group; however, these values were not significantly different than those of the CD28% High group (P=0.35). These minor disparities are likely anomalies arising from the small numbers of subjects, particularly as no similar patterns were evident in either Cohort B or the aggregate study population (see Tables 3 and 4).

TABLE 3
Characteristics of Cohort B (all have BOS)

| | CD28% High | CD28% Low |
|---|---|---|
| n | 19 | 11 |
| Age (years) | 48 ± 3 | 53 ± 5 |
| % Male | 26 | 36 |
| Months since Transplant | 100 ± 13 | 74 ± 16 |
| Transplantation Type -n (%) | | |
| SL | 9 (47) | 6 (55) |
| DL | 8 (42) | 5 (45) |
| HL | 2 (10) | 0 (0) |
| Pretransplant Diagnoses -n (%) | | |
| COPD | 4 (21) | 2 (18) |
| Pulmonary Fibrosis | 3 (16) | 2 (18) |
| Cystic Fibrosis | 5 (26) | 3 (27) |
| PA Hypertension | 6 (32) | 3 (27) |
| Sarcoidosis/Scleroderma | 1 (5) | 1 (9) |
| Recipients with Previous | 8 (42) | 8 (73) |
| CMV -n (%) | | |
| Interval Since CMV (months) | 39 ± 14 | 41 ± 15 |
| ACR-n (%) | 4 (21) | 2 (18) |
| ACR Grade | 1.8 ± 0.5 | 1.5 ± 0.5 |
| Induction Regimens - (%) | | |
| Glucocorticosteroid/ | 16 (84) | 8 (73) |
| Azathioprine | | |
| Daclizumab | 0 | 0 |
| RATG | 1 (5) | 1 (9) |
| Alemtuzumab | 2 (11) | 2 (18) |
| Donor:Recipient MHC Mismatches | | |
| Class I | 3.3 ± 0.2 | 2.4 ± 0.3 |
| Class II | 3.2 ± 0.3 | 2.6 ± 0.3 |

TABLE 4
Characteristics of Aggregate Study Population (Cohorts A + B)

| | CD28% High | CD28% Low |
|---|---|---|
| n | 46 | 19 |
| Age (years) | 49.2 ± 1.8 | 52.2 ± 2.9 |
| % Male | 37 | 47 |
| Number (%) with BOS | 24 (56%) | 16 (84)* |
| Months since Transplant | | |
| Transplantation Type -n (%) | | |
| SL | 22 (48) | 10 (53) |
| DL | 20 (43) | 9 (47) |
| HL | 4 (9) | 0 (0) |
| Pretransplant Diagnoses -n (%) | | |
| COPD | 17 (37) | 9 (47) |
| Pulmonary Fibrosis | 11 (24) | 3 (16) |
| Cystic Fibrosis | 8 (17) | 3 (16) |
| PA Hypertension | 6 (13) | 2 (11) |
| Sarcoidosis | 3 (7) | 1 (5) |
| Scleroderma | 1 (2) | 1 (5) |
| Recipients with Previous CMV -n (%) | 15 (33) | 11 (58) |
| Interval Since CMV (months) | 32 ± 8 | 32 ± 10 |
| ACR-n (%) | 12 (26) | 4 (21) |
| ACR Grade | 1.8 ± 0.3 | 1.5 ± 0.3 |
| Induction Regimens - (%) | | |
| Glucocorticosteroid/Azathioprine | 30 (65) | 15 (79) |
| Daclizumab | 4 (9) | 0 (0) |
| RATG | 7 (15) | 2 (11) |
| Alemtuzumab | 5 (11) | 2 (11) |
| Donor:Recipient MHC Mismatches | | |
| Class I | 3.4 ± 0.2 | 3.5 ± 0.2 |
| Class II | 2.7 ± 0.2 | 2.6 ± 0.2 |

For Tables 3 and 4, the following abbreviations were used. CD28% High denotes those recipients in whom >90% of their circulating CD4 T-cells also expressed CD28, whereas recipients in whom CD28 was expressed on fewer than 90% of their circulating CD4 T cells are denoted as CD28% Low. BOS denotes bronchiolitis obliterans syndrome. SL, DL, HL describes Single Lung, Double Lung, and Heart Lung transplantations, respectively; COPD=chronic obstructive pulmonary disease; PA=pulmonary artery; Previous CMV describes subjects who had CMV infections prior to these CD4 T-cell assays; and Interval Since CMV describes the time interval between these infections and the CD4 assays. ACR denotes acute cellular allograft rejection at the time of CD4 assays. RATG denotes rabbit antithymocyte globulin. In Table 3, three of these subjects (CD28% High=2, CD28% Low=1) were taking rapamycin at the time of their CD4 assay. None of the comparisons were statistically significant. In Table 4, the asterisk (*) denotes p=0.016. None of the other comparisons were statistically significant.

CD4+CD28null T Cells from Recipients Have Pathogenic Characteristics. A series of investigations were performed to begin characterizations of the unusual CD4+CD28null T cells. Because few of the No-BOS recipients had appreciable proportions of these cells, subsequent subject enrollments were limited to sequential, outpatient lung transplant patients seen in clinic who had preexistent BOS (Cohort B). All of these recipients fulfilled previously described inclusion criteria (as described above in the Methods section), were otherwise consecutively recruited, and laboratory tests were interpreted by investigators blinded to identities, demographic details, treatments, clinical courses, or other subject characteristics (see Table 4). Based on analogous studies in autoimmune and other patients with chronic inflammatory diseases (Vallejo A N, et al, Trends Mol Med 2004; 10:119-24; Vallejo A N. Rev 2005; 205:158-69; van Leeuwen E M M, et al. J Immunol 2004; 173:1834-41; Goronzy J J, et al. Arthritis Rheum 2004; 50:43-54; Raffeiner B, et al. Arthritis Res Ther 2005; 7: R1312-R1320; Zal B, et al. Circulation 2004; 109:1230-35; Lamprecht P, et al. Thorax 2001; 56: 751-57; Komocsi A, et al. Am J Pathol 2002; 160:1717-24; Snyder M R, et al. J Immunol 2002; 168:3839-46; Liuzzo G, et al. Circulation 2000; 101:2883-88; Hirokawa M, et al. Bone Marrow Transplant 2001; 27:1095-1100; Schirmer M, et al. J Immunol 1998; 161:1018-25; and Pawlik A, et al. Transplant Proc 2003; 35:2902-04), the CD4+CD28null cells were expected to be highly activated. A greater proportion of the CD4+CD28null (36.5±6.9%) did express major histocompatibility complex antigen class II than the corresponding autologous CD4+CD28+ cells (11.4±3.7%) (n=16; P=0.006), but the latter more frequently expressed CD25, another marker of T-cell activation (FIG. 1D). This latter finding led us to speculate that the proportion of regulatory CD4 T cells (Tregs), defined by expression of FoxP3 (in turn, associated with CD25 expression, see Rutella S, Lemoli R M. Regulatory T cells and tolerogenic dendritic cells: from basic biology to clinical applications. Immunol Lett 2004; 94:11-26), may also be diminished among CD4+CD28null cells. Measures within a recent, randomly selected, consecutive subpopulation of Cohort B subjects showed that FoxP3+ cells among the CD4+CD28null are significantly less frequent than in the autologous CD4+CD28+ lymphocytes (FIG. 1D) or CD4+CD28+ T cells of recipients with No-BOS (13.7±2.5%; n=7; P=0.008). CD4+CD28null cells derived from patients with autoimmune and other chronic immunologic diseases also frequently produce the cytotoxic mediators, perform and granzyme B, in striking contrast to normal CD4 T cells that do not elaborate these substances (Vallejo A N. Immunol Rev 2005; 205:158-69 and Goronzy J J, et al. Prognostic markers of radiographic progression in early rheumatoid arthritis. Arthritis Rheum 2004; 50:43-54). The assays here confirm that CD4+CD28null cells of lung transplant recipients with BOS also produce these potentially pathogenic mediators (FIG. 1D).

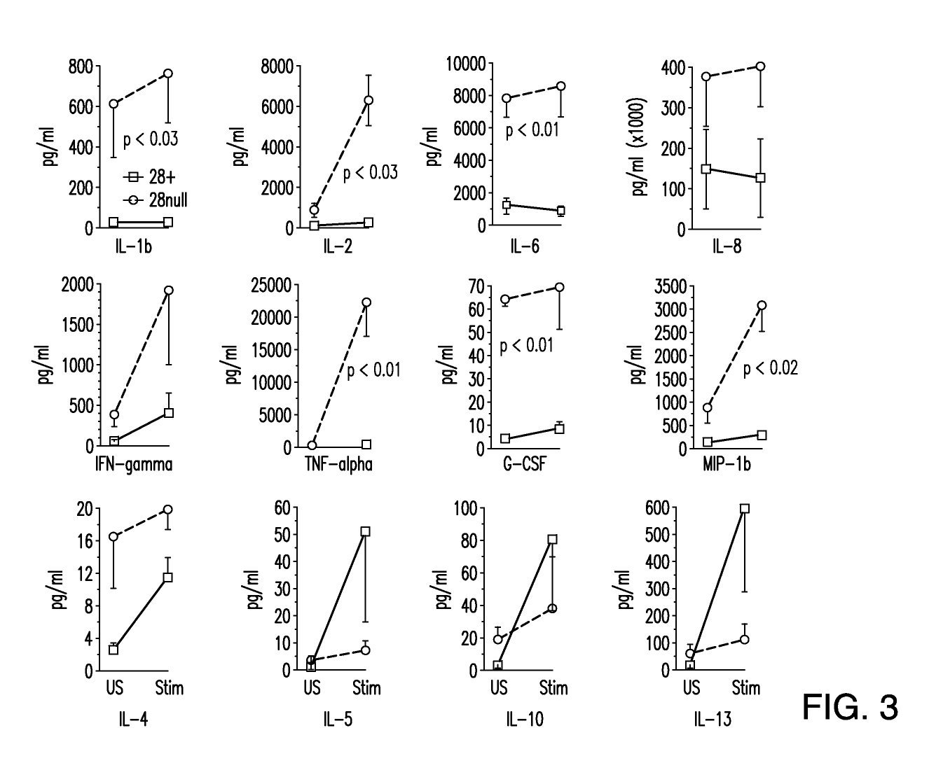

CD4+CD28null T Cells of Recipients with BOS Produce Proinflammatory Cytokines. Differential cytokine production of autologous CD4+CD28+ and CD4+CD28null cells were apparent in multiplex analyses. CD4+CD28null cells generally elaborated much greater amounts of proinflammatory and Th1 mediators under basal conditions, and, in many cases, this production was strikingly increased by activation after T-cell antigen receptor (TCR) cross-linking with anti-CD3 antibody (FIG. 3). Conversely, and with the exception of IL-4, CD4+CD28null cell elaborations of Th2 cytokines, notably including putatively immunosuppressive IL-10 (Conti P, et al. IL-10, an inflammatory/inhibitory cytokine, but not always. Immunol Lett 2003; 86:123-129), were reduced compared with production by autologous CD4+CD28+ cells. FIG. 3 shows cytokine elaborations by autologous CD4 subpopulations of recipients with chronic rejection. The initial (left) data point in each series represents control unstimulated (US) condition, whereas the second (right) data point delineates production of cells after stimulation with plate-bound anti-CD3 antibody (Stim). These paired specimens (US and Stim) are also connected by lines. CD4+CD28null cells from transplant recipients with BOS (open circles) tend to elaborate greater amounts of proinflammatory and Th1 cytokines (top two rows), whereas CD4+CD28+(open squares with paired specimens connected by solid lines) have an apparent Th2 bias (bottom row) (n=5 randomly selected, consecutive specimens in each).

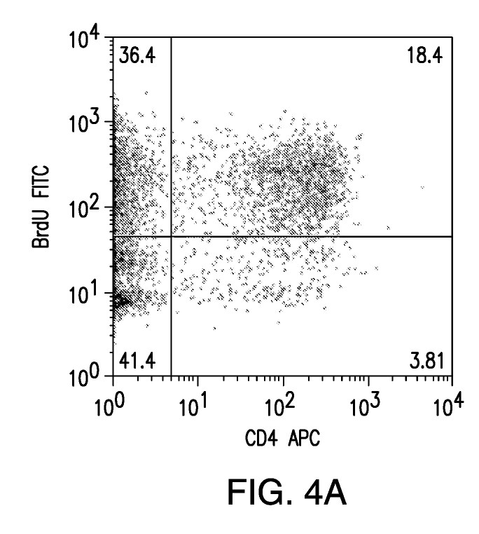

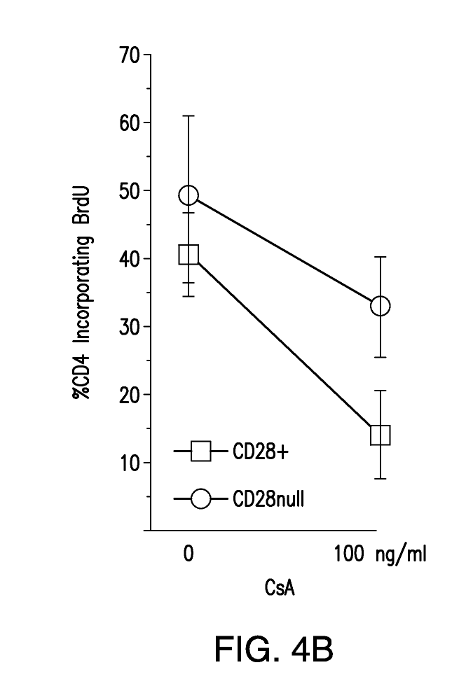

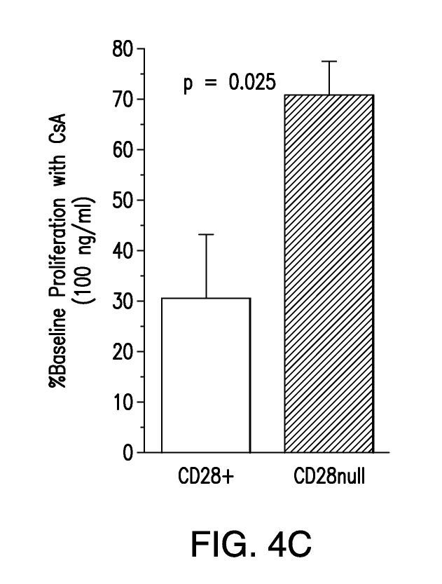

CD4+CD28null T Cells from Recipients Are Relatively Resistant to Antiproliferative Effects of Cyclosporine A. CR typically develops and often progresses despite intense immunosuppression (Boehler A, Estenne M. Eur Respir J 2003; 22:1007-1018; Trulock E P, et al. J Heart Lung Transplant 2006; 25:880-892; and Estenne M, Hertz M I. Am J Respir Crit. Care Med 2002; 166:440-444). It was hypothesized that, if CD4+CD28null lymphocytes are indeed disproportionately pathogenic, as suggested by their occurrence primarily among recipients with BOS, as well as their seemingly pathogenic phenotype (FIGS. 1D and 3), these cells might also be relatively resistant to immunosuppressant medications. Using cyclosporine A (CsA) as a prototype calcineurin inhibitor, and an agent that provides a novel exposure for these specimens, the ability of the respective CD4 subpopulations to proliferate after TCR stimulation with plate-bound anti-CD 3 monoclonal antibody was examined (thereby replicating T-cell engagements with antigens/alloantigens) while in the presence of therapeutic drug concentrations. Although freshly isolated autologous CD4+CD28+ and CD4+CD28null lymphocytes proliferated comparably with in vitro stimulation in control wells (no cyclosporine), proliferation of the CD4+CD28null cells was comparatively less inhibited by cyclosporine in every subject (FIG. 4). Moreover, these results are not confounded by pharmacologic effects of the small amount of cyclosporine carrier (solvent) in these cultures, as addition of pure carrier, in concentrations of more than 100-fold higher than that used in these drug resistance studies, had no effect on T-cell proliferation. Preliminary findings in small numbers of subjects (n=2) also indicate that CD4+CD28null T cells are similarly relatively resistant to tacrolimus.

FIGS. 4A-C show the effects of cyclosporine A on proliferation of CD4 T-cell subpopulations isolated from lung transplant recipients with chronic rejection. FIG. 4A provides an illustrative example of proliferation quantitation as assessed by bromodeoxyuridine (BrdU) incorporation and flow cytometry. Viable cells were gated and respective populations of CD4 T cells (in this case CD4+CD28null) that incorporated BrdU were determined. In this example, ˜83% of the CD4+CD28null incorporated BrdU. All CD4+ cells had been previously depleted from these cultures, except the particular CD4 T-cell subpopulation being evaluated (either CD28+ or CD28null) (as described above in the Methods section). FIG. 4B shows proliferations, ascertained by the percentages of respective autologous CD4 T-cell subpopulations that incorporated BrdU after stimulation by plate-bound anti-CD3 antibody, were approximately equivalent in both CD4 T-cell groups in the absence of cyclosporine (control). In every recipient with BOS tested (n=6), however, proliferation among their CD4+CD28+ cells were decreased more by cyclosporine (100 ng/ml) than were the proliferations of their autologous CD4+CD28null cells. (FIG. 4C shows that the aggregate decrement of proliferation in the presence of cyclosporine, relative to control (no cyclosporine) values, was significantly greater among the CD4+CD28+ cells.

CD28 Down-Regulation on CD4 T Cells of Recipients Is Associated with Pulmonary Function. If the unusual CD4+CD28null cells are pathogenic, the extent of CD28 down-regulation among particular recipients could possibly be associated with clinical manifestations of allograft injury. These analyses were performed in the initial, consecutively recruited subject population (Cohort A) to avoid cryptic ascertainment biases.

As single cross-sectional “snapshots,” values of CD28% had little correlation with concurrent pulmonary function, given that baseline values of FEV1, performed at the time of the initial CD4 assays, were similar among CD28% High and CD28% Low populations (see Tables 5 and 6).

TABLE 5
Pulmonary Function of Cohort A Subjects

| | CD28% High | CD28% Low |
|---|---|---|
| N | 27 | 8 |
| Baseline FEV₁ (L) | 1.9 ± 0.1 | 2.0 ± 0.3 |
| FEV₁% predicted values | 65.0 ± 4.4 | 66.5 ± 8.5 |
| % Max. Post-Tx FEV₁ | 85.9 ± 3.8 | 77.4 ± 6.8 |
| FEV₁ (L) among Non-BOS | 2.0 ± 0.2 | 2.7 ± 0.7 |
| FEV₁ (L) among BOS | 1.3 ± 0.2 | 1.7 ± 0.1 |
| BOS GRADE: n (%) | | |
| 0 | 17 (63) | 3 (38) |
| 0-p | 5 (19) | 0 |
| 1 | 2 (7) | 3 (38) |
| 2 | 0 | 1 (13) |
| 3 | 3 (11) | 1 (13) |

TABLE 6
Pulmonary Function of Cohort B Subjects (all have BOS)

| | CD28% High | CD28% Low |
|---|---|---|
| N | 19 | 11 |
| Baseline FEV₁ (L) | 1.2 ± 0.1 | 1.1 ± 0.2 |
| FEV₁% predicted values | 44.2 ± 4.2 | 41.1 ± 4.9 |
| % Max. Post-Tx FEV₁ | 51.2 ± 4.4 | 50.4 ± 6.7 |
| BOS GRADE: n (%) | | |
| 1 | 5 (26) | 3 (27) |
| 2 | 6 (32) | 3 (27) |
| 3 | 8 (42) | 5 (45) |

For Tables 5 and 6, “CD28% High” denotes those recipients in which ≧90% of their circulating CD4 T-cells also expressed CD28, whereas recipients in whom CD28 was expressed on <90% of their circulating CD4 T-cells are denoted as “CD28% Low.” Baseline FEV1 denotes values at time of initial CD28 assay. % Max FEV1 Post-Tx describes FEV1 at the time of assay, relative to the mean maximal FEV1 of two determinations (>3 weeks apart) that occurred after transplantation (Estenne M, et al. J Heart Lung Transplant 2002; 21:297-310), but before the CD28 assays. BOS denotes bronchiolitis obliterans syndrome and Grades of same are defined elsewhere (Estenne M, et al. J Heart Lung Transplant 2002; 21:297-310). For both Tables 5 and 6, none of the intergroup comparisons were statistically significant.

It seems likely that these simple assessments may be prone to confounding by the heterogeneity within the populations (e.g., double- vs. single-lung transplantations, obstruction vs. restriction in native single lungs), as well as differences of subject body sizes, ages, genders, etc. In addition, variances of pulmonary function values within groups (“noise”) were overtly increased by the presence of BOS subjects (with diminished FEV1) among the CD28% High subjects and, conversely, No-BOS recipients (with normal expiratory flow) among the CD28% Low subjects (see Table 5).

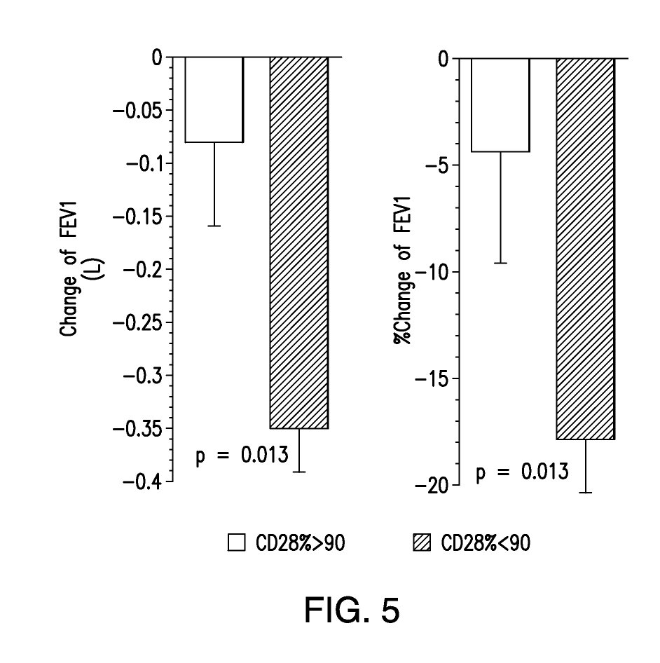

However, CD28% values did associate with future changes of pulmonary function among these subjects, as decrements of FEV1 at subsequent routine 6-month (surveillance) spirometry were more than fourfold greater among the CD28% Low recipients (FIG. 5). FIG. 5 shows that decrements of FEV1 were significantly greater among Cohort A recipients with CD4+CD28+/CD4total values less than 0.9 (CD28% Low; n=7) compared with recipients with CD4+CD28+/CD4total values of 0.9 or greater (CD28% High; n=27). Routine surveillance pulmonary function tests (PFTs) were performed 6.0 (SEM, ±0.5) 18 months and 6.6 (SEM, ±1.3) months after CD28 determinations (CD28% Low and CD28% High, respectively). One CD28% Low subject did not have pulmonary function measured after her initial CD4 assay due to severe allograft dysfunction (BUS) and later demise.

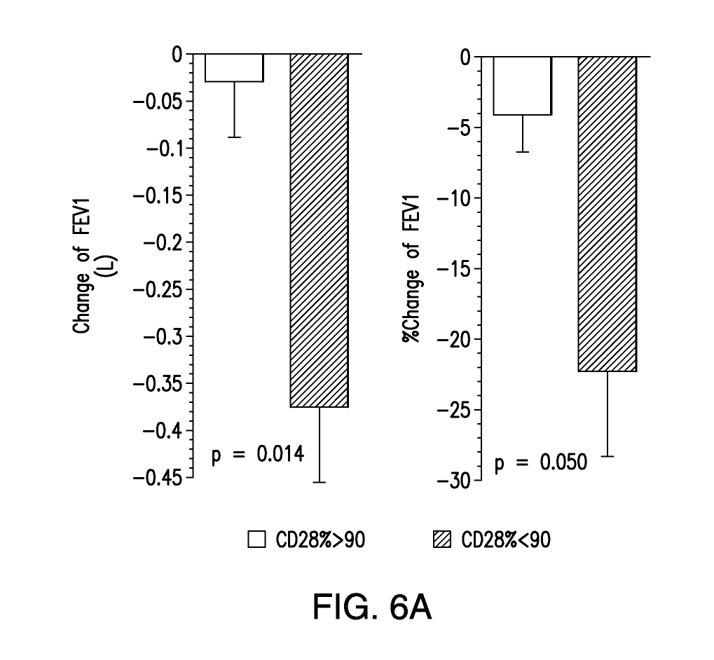

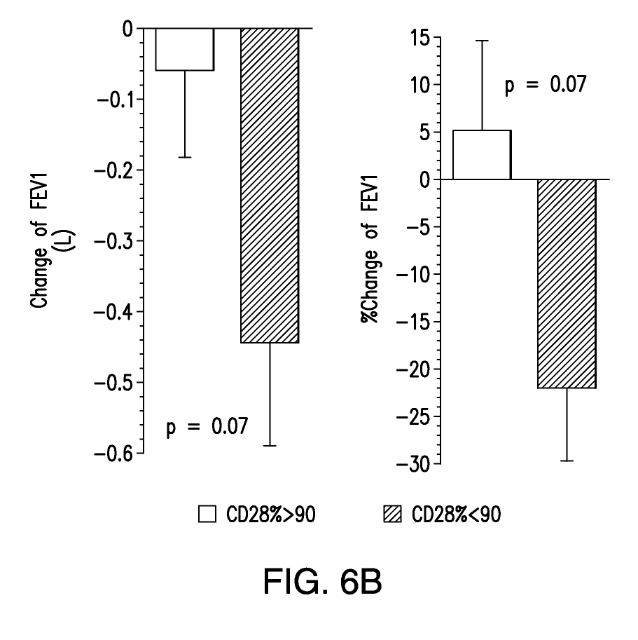

Because an increased proportion of the CD28% Low subjects had BOS, it could be postulated that the greater pulmonary function deterioration in this population could possibly be attributable to confounding by the expected progression of their graft dysfunction. Accordingly, post hoc analyses limited to the recipients with BOS were performed. One of the five original CD28% Low subjects with BOS rapidly deteriorated after her CD4 assay, and did not have further pulmonary function testing. Deteriorations of expiratory airflow among the four (4) BOS CD28% Low subjects were much greater than in the five (5) BOS CD28% High subjects, and intergroup differences remained significant, despite the small population sizes (see FIG. 6A).

Intergroup differences in the extent of FEV1 changes between CD28% High and CD28% Low groups also seemed evident 1 year after the CD28 assays (FIG. 6B). FIG. 6A shows subsequent decrements of FEV1 were significantly greater among Cohort A CD28% Low with BOS compared to the CD28% High recipients with BOS. Pulmonary function was measured at routine, surveillance evaluations 6 months after their CD4 assays (see also the brief description of FIG. 5). FIG. 6B shows that subsequent decrements of FEV1 were significantly greater among Cohort A CD28% Low (n=5) compared to CD28% High (n=22) at 11.8+0.7 and 11.9+0.4 months after their initial CD28 measurement, respectively. Subject drop out in both populations was due to interval deaths or development of severe graft dysfunction that precluded these later pulmonary function tests. However, interval drop-outs from deaths, re-transplantations, or severe allograft dysfunction that precluded PFTs resulted in smaller numbers of evaluable subjects, particularly among the CD28% Low group.

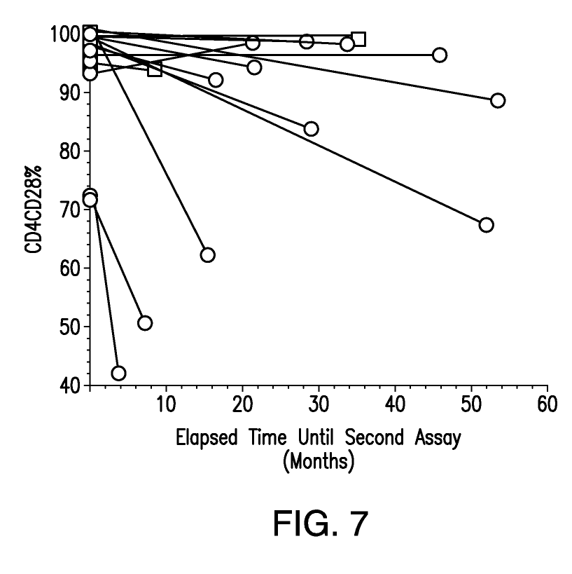

Replicate CD28 assays were performed in 14 of the Cohort A recipients during repeated, routine clinic visits (FIG. 7). FIG. 7 shows changes in CD28 expression by CD4 T-cells of recipients (CD28%) over time, among 14 Cohort A subjects with replicate assays. Months denote intervals between first and second CD28 determinations. Open squares denote values of recipients without BOS at the time of these assays, whereas open circles denote subjects with BOS at these respective assay time points. Nine of these recipients were No-BOS at the time of their initial assays, but seven of these developed BOS prior to their second CD28% measurements.

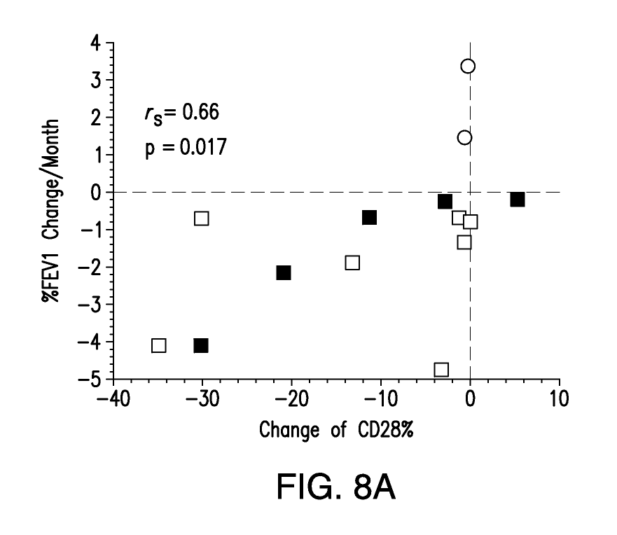

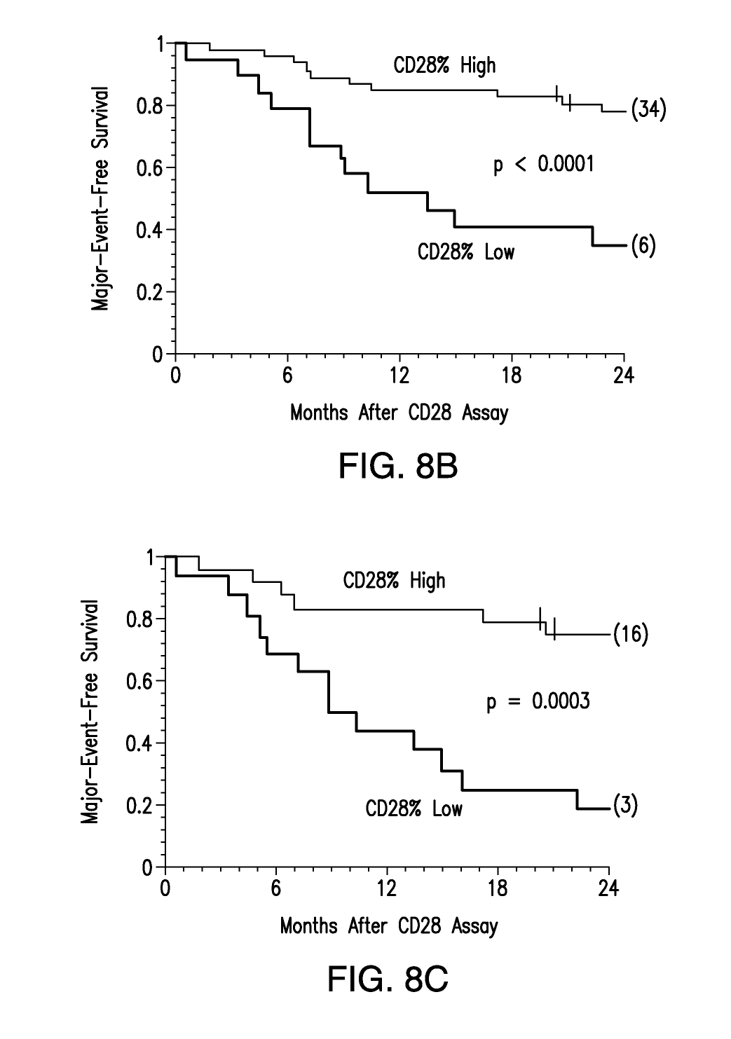

These recruitments too were essentially random selections resulting from recipient return appearances in outpatient clinic, continued absence of exclusion criteria, and willingness to participate by providing another blood sample. Changes of CD28% over time in these serial, replicate measurements correlated with corresponding pulmonary function alterations among the individual subjects (FIG. 8A). FIG. 8A shows an association of changes in FEV1 (as percentages of initial values) versus changes in CD28% among those Cohort A subjects who were available and consented to replicate studies. Open squares denote those recipients who were Non-BOS at the time of their first T-cell assay, but had progressed to BOS by the time of their second determination; closed squares denote those recipients who had BOS at both CD28% determinations; and open circles represent recipients who were non-BOS throughout.

CD28 Down-Regulation on CD4 T Cells of Recipients Is Associated with Adverse Clinical Outcomes. Inexorable allograft injury in lung transplantation recipients eventually leads to pulmonary retransplantation and/or death (Boehler A, Estenne M. Eur Respir J 2003; 22:1007-1018; Trulock E P, et al. J Heart Lung Transplant 2006; 25:880-892; and Estenne M, Hertz M I. Am J Respir Crit. Care Med 2002; 166:440-444). Given the apparent associations between the extent of CD28 expression and pulmonary function (FIGS. 5 and 8A), the possibility was evaluated that CD4 T-cell phenotype abnormalities could also be linked with overall clinical outcome. Survival analyses of the subjects was performed, again dichotomously stratified by their CD28 expressions (i.e., CD28% High vs. CD28% Low).

Product-limit analyses demonstrated that major events (death or retransplantation) during the 2 years after their first CD4 T-cell assays occurred much more frequently among CD28% Low subjects compared with CD28% High recipients (FIG. 8B). FIG. 8B shows survival curves showing cumulative freedom from major adverse events of CD28% High (n=46) and CD28% Low (n=19) among all recipients in the aggregate subject populations (both Cohorts A and B). Tick marks denote interval-censored events, and numbers in parentheses at end of the survival curves denote remaining, unafflicted subjects who were censored at 24 months of observation.

These differences in outcome seem unlikely to be simply attributable to the higher prevalence of BOS, with inherent increased morbidity and mortality (Boehler A, Estenne M. Eur Respir J 2003; 22:1007-1018; Trulock E P, et al. J Heart Lung Transplant 2006; 25:880-892; and Estenne M, Hertz M I. Am J Respir Crit. Care Med 2002; 166:440-444), among the CD28% Low subjects (Table 2). To begin with, irrespective of the diagnoses at the time of their CD28% assays here, every subject who suffered a major adverse event had progressed to severe BOS before the occurrences of their serious complications (see Tables 7 and 8).

TABLE 7
Major Adverse Events among Individual Recipients

| | | | Month | | |
|---|---|---|---|---|---|
| | Diagnosis | | after | Diagnosis | |
| Cohort | (initial) | CD28% | assay | (at event) | Event/cause |
| A | No- BOS | High | 9.4 | BOS | Dead/Graft Failure |
| A | No- BOS | High | 7.3 | BOS | Dead/BOS + |
| | | | | | Pneumonia |
| A | BOS | Low | 13.5 | BOS | Dead/Graft Failure |
| A | BOS | Low | 5.2 | BOS | Dead/BOS + |
| | | | | | Pneumonia |
| A | BOS | Low | 22.3 | BOS | Retransplantation |
| | | | | | for graft failure |
| A | No- BOS | High | 22.8 | BOS | Retransplantation |
| | | | | | for graft failure |
| A | No- BOS | High | 10.5 | BOS | Retransplantation |
| | | | | | for graft failure |
| A | No- BOS | Low | 15.0 | BOS | Retransplantation |
| | | | | | for graft failure |
| A | No- BOS | High | 4.8 | BOS | Retransplantation |
| | | | | | for graft failure |
| B | BOS | Low | 0.6 | BOS | Dead/Graft Failure |
| B | BOS | Low | 4.5 | BOS | Dead/Graft Failure |
| B | BOS | Low | 3.2 | BOS | Dead/Graft Failure |
| B | BOS | High | 20.7 | BOS | Dead/BOS + |
| | | | | | Pneumonia |
| B | BOS | Low | 7.2 | BOS | Dead/Graft Failure |
| B | BOS | Low | 8.9 | BOS | Dead/Graft Failure |
| B | BOS | High | 17.2 | BOS | Dead/Graft Failure |
| B | BOS | Low | 16.1 | BOS | Dead/Graft Failure |
| B | BOS | Low | 3.5 | BOS | Retransplantation |
| | | | | | for graft failure |
| B | BOS | Low | 10.4 | BOS | Retransplantation |
| | | | | | for graft failure |
| B | BOS | High | 7.0 | BOS | Retransplantation |
| | | | | | for graft failure |
| B | BOS | High | 1.8 | BOS | Retransplantation |
| | | | | | for graft failure |
| B | BOS | High | 1.5 | BOS | Retransplantation |
| | | | | | for graft failure |

For Table 7, clinical features are shown for recipients who suffered major adverse events (death or retransplantation). CD28% values are results of initial CD4 assays. CD28% High=CD28%>90; Low=CD28%<90. “Diagnosis (initial)” denotes categorization at time of initial CD28 assay, whereas “Diagnosis (at event)” describes established diagnoses at time of major adverse event. “Months after assay” denotes elapsed time from CD28 assay until adverse event.

TABLE 8
Pulmonary Function Proximate to Major Adverse Events

| | | FEV₁% | Months prior to |
|---|---|---|---|
| | FEV₁ (L) | Predicted | Adverse Event |
| Recipients who died | 0.69 ± 0.08 | 24.0 ± 2.9 | 4.7 ± 0.8 |
| Retransplanted Recipients | 0.71 ± 0.10 | 24.4 ± 2.9 | 2.7 ± 0.6 |

For Table 8, “Months Prior to Adverse Event” denotes interval between last pulmonary function tests and occurrence of adverse event. See also Table 7.

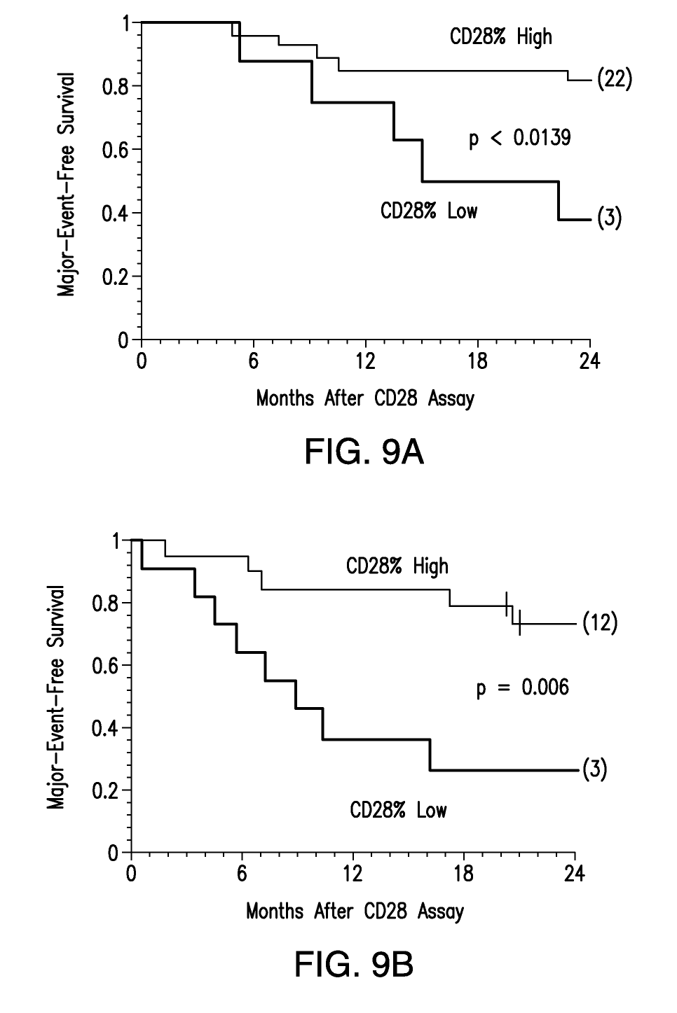

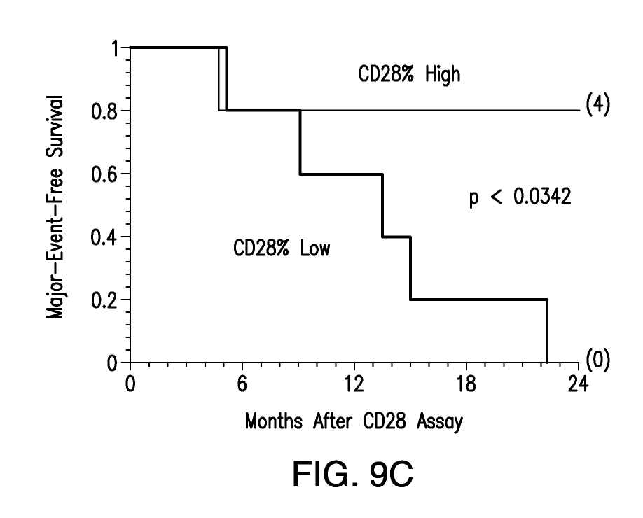

Moreover, survival analyses limited to recipients with BOS already present at the time of their initial CD28% assays show even greater intergroup differences, with still considerable statistical significance, despite smaller numbers of subjects (FIG. 8C). FIG. 8C shows survival curves showing cumulative freedom from major adverse events of CD28% High (n=24) and CD28% Low (n=16) among all BOS recipients in the aggregate subject populations (both Cohorts A and B). The event-free survival advantage of CD28% High was also evident in subpopulation analyses of individual subject Cohorts (FIGS. 9A-C). FIG. 9A shows actuarial survival curves showing cumulative freedom from major adverse events (retransplantation or death) of CD28% High (n=27) and CD28% Low (n=8) among Cohort A recipients (both No-BOS and BOS), using CD28% values from the recipients first assays. Numbers in parenthesis at end of the survival curves denote remaining, unafflicted subjects that were censored at 24 months of observation. FIG. 9B shows actuarial survival curves showing cumulative freedom from major adverse events (retransplantation or death) of CD28% High (n=19) and CD28% Low (n=11) among Cohort B recipients (all of whom had BOS). Tick marks denote interval censored subjects. FIG. 9C shows actuarial survival curves showing cumulative freedom from major adverse events (retransplantation or death) of CD28% High and CD28% Low among Cohort A recipients with BOS (n=5 in each group).

Discussion

These data demonstrate that CD28 down-regulation on peripheral CD4 T cells of lung allograft recipients is associated with subsequent allograft dysfunction. The CD4+CD28null cells seen in greater proportions among recipients with BOS exhibit unusual phenotypes, including discordant expression of activation markers, frequent production of cytotoxic mediators, and elaboration of large amounts of proinflammatory mediators, but decreased Treg marker FoxP3 expression and relative cyclosporine resistance. Altogether, these findings suggest that CD4+CD28null cells are a pathogenic T-cell subpopulation involved in allograft injury.

Nearly all normal human CD4 T lymphocytes express CD28 on their cell surfaces (Sharpe A H, Freeman G J. The B7-CD28 superfamily. Nat Rev Immunol 2002; 2:116-126), and findings that proportions of circulating CD4+CD28null T cells are increased are distinctly abnormal (Vallejo A N, et al. Trends Mol Med 2004; 10:119-24; Vallejo A N. Rev 2005; 205:158-69; van Leeuwen E M M, et al. J Immunol 2004; 173:1834-41; Goronzy J J, et al. Arthritis Rheum 2004; 50:43-54; Raffeiner B, et al. Arthritis Res Ther 2005; 7: R1312-R1320; Zal B, et al. Circulation 2004; 109:1230-35; Lamprecht P, et al, Thorax 2001; 56: 751-57; Komocsi A, et al. Am J Pathol 2002; 160:1717-24; Snyder M R, et al. J Immunol 2002; 168:3839-46; Liuzzo G, et al. Circulation 2000; 101:2883-88; Hirokawa M, et al. Bone Marrow Transplant 2001; 27:1095-1100; Schirmer M, et al. J Immunol 1998; 161:1018-25; and Pawlik A, et al. Transplant Proc 2003; 35:2902-04). Cognate interactions of T-cell CD28 with ligands CD80 and CD86 provide a “second signal” for lymphocyte activation, in conjunction with the “primary signals” mediated by TCR engagements with complexes of antigen—major histocompatibility complex. Nonetheless, neither previously activated (“memory”) CD4 nor naïve CD8 T cells require CD28 for activation, and other costimulatory molecules can also provide the necessary second signal for initial activations of naïve CD4 cells (Sharpe A H, Freeman G J. Nat Rev Immunol 2002; 2:116-126; Fontenot A P, et al. CD28 costimulation independence of target organ versus circulating memory antigen-specific CD4 T cells. J Clin Invest 2003; 112: 776-784). CD28 down-regulation on CD4 T cells in humans is a hallmark of ongoing, chronic adaptive immune responses, and has been frequently noted in patients with autoimmune and other chronic inflammatory diseases (Vallejo A N, et al. Trends Mol Med 2004; 10:119-24; Vallejo A N. Rev 2005; 205:158-69; van Leeuwen E M M, et al. J Immunol 2004; 173:1834-41; Goronzy J J, et al. Arthritis Rheum 2004; 50:43-54; Raffeiner B, et al. Arthritis Res Ther 2005; 7: R1312-R1320; Zal B, et al. Circulation 2004; 109:1230-35; Lamprecht P, et al. Thorax 2001; 56: 751-57; Komocsi A, et al. Am J Pathol 2002; 160:1717-24; Snyder M R, et al. J Immunol 2002; 168:3839-46; Liuzzo G, et al. Circulation 2000; 101:2883-88; Hirokawa M, et al. Bone Marrow Transplant 2001; 27:1095-1100; Schirmer M, et al. J Immunol 1998; 161:1018-25; and Pawlik A, et al. Transplant Proc 2003; 35:2902-04).

Previous studies have indicated that CD4+CD28null cells derived from patients with autoimmune diseases are highly pathogenic. These particular cells autonomously elaborate IFN-γ (Komocsi A, et al. Am J Pathol 2002; 160:1717-1724) (see also FIG. 3), express natural killer cell killer immunoglobulin-like receptors (Snyder M R, et al. J Immunol 2002; 168:3839-46), and frequently produce cytolytic mediators (van Leeuwen E M M, et al. J Immunol 2004; 173:1834-1841; Raffeiner B, et al. Arthritis Res Ther 2005; 7: R1312-R1320) (see also FIG. 1D). The CD4+CD28null cells of autoimmune patients are markedly oligoclonal, thus demonstrating that they are daughter progeny of repeated antigen-driven proliferations (Vallejo A N, et al. Trends Mol Med 2004; 10:119-24; Vallejo A N. Rev 2005; 205:158-69; Liuzzo G, et al. Circulation 2000; 101:2883-88; Hirokawa M, et al. Bone Marrow Transplant 2001; 27:1095-1100; Schirmer M, et al. J Immunol 1998; 161:1018-25). In addition, the CD4+CD28null clones bear idiotypic TCR sequences that are also present within autologous “normal” CD4+CD28+ cells, showing that the former are phenotypic variants of the latter, and that both populations share common progenitors (Hirokawa M, et al. Bone Marrow Transplant 2001; 27:1095-1100; Colombatti A, et al. Age-related persistent clonal expansions of CD28-cells: phenotypic and molecular TCR analysis reveals both CD4+ and CD4+CD8+ cells with identical CDR3 sequences. Clin Immunol Immunopathol 1998; 89:61-70). CD4+CD28null cell specificity for autoantigens has also been demonstrated (Zal B, et al. Circulation 2004; 109:1230-35), and CD28null quantitation may correlate with clinical progression of autoimmune disease (Goronzy J J, et al. Arthritis Rheum 2004; 50:43-54).

The cognate recognition of peptide alloantigens (notably including, but not limited to polymorphic major histocompatibility antigens) by recipient lymphocytes is widely recognized as an early and critical step in the cascade of responses leading to CR (Duncan S R, et al. Am J Respir Crit. Care Med 2002; 165:1439-1444; Krensky A M, et al. N Engl J Med 1990; 322:510-517; Richards D M, et al. J Immunol 2004; 172:3469-3479). CD4 T cells have pleotropic effector capabilities that can account for the allograft injuries associated with chronic rejection, either directly or by mediator elaborations that activate and/or recruit secondary tiers of downstream effector cells and other processes (Monaco C, et al. Curr Drug Targets Inflamm Allergy 2004; 3:35-42). In the particular case of human lung transplantation, nonimmunologic injuries (e.g., graft ischemia and various infections) have also been implicated in allograft injury (Boehler A, Estenne M. Eur Respir J 2003; 22:1007-18; Trulock E P, et al. J Heart Lung Transplant 2006; 25:880-92; Estenne M, Hertz M I. Am J Respir Crit. Care Med 2002; 166:440-44; Neuringer I P, et al. Obliterative bronchiolitis or chronic lung allograft rejection: a basic science review. J Heart Lung Transplant 2005; 24:3-19). Although multiple distinct injury pathways may converge to produce OB, it is perhaps also possible that many of these injuries ultimately enhance immunologic recognition of the allograft by causing increased production or presentation of alloantigens and/or proinflammatory mediators, and thereby facilitate and/or promote the injurious adaptive immune responses that lead to dysfunction of the transplanted lung(s) (White S, Rosen A. Apoptosis in systemic lupus erythmatosus. Curr Opin Rheumatol 2003; 15:557-562; El-Sawy T, et al. Early T-cell response to allografts occurring prior to alloantigen priming up-regulates innate-mediated inflammation and graft necrosis. Am J Pathol 2004; 165:147-157; Haynes L D, et al. CMV-infected allogeneic endothelial cells initiate responder and bystander donor HLA class I release via the metalloproteinase cleavage pathway. Hum Immunol 2005; 66:211-221; Burne-Taney M J, et al. Persistent renal and extrarenal immune changes after severe ischemic injury. Kidney Int 2005; 67: 1002-1009; Daud S A, et al. The impact of immediate primary lung allograft dysfunction on bronchiolitis obliterans syndrome. Am J Respir Crit Care Med 2007; 175:507-513).

The pathogenic potential of the CD4+CD28null cells appears to be considerable, and, in many respects, is highly comparable to that reported for analogous cells from patients with autoimmune conditions and other patients with chronic inflammatory diseases (Vallejo A N, et al. Trends Mol Med 2004; 10:119-124; Vallejo A N. Immunol Rev 2005; 205:158-169). Perform and granzyme B production by the CD4+CD28null cells (FIG. 1D) may explain previous observations of CD4 T-cell cytoxicity among lung transplant recipients (Poindexter N J, et al. Function of CD4+ cytolytic T lymphocytes in lung allografts. Transplant Proc 1999; 31:195-96). The pattern of cytokine production by CD4+CD28null cells shows that mediators that generally initiate and amplify immune responses were typically produced in much greater quantities by these particular lymphocytes than in their autologous CD4+CD28+ counterparts (FIG. 3). These assays also show that most Th2 cytokine production by the CD4+CD28null subpopulation are, typically, relatively little increased upon TCR stimulation (notably excepting IL-4). The comparatively lesser production of IL-10 by the CD4+CD28null cells (relative to autologous CD4+CD28+ cells) may have singular biologic importance, given the possible role of this cytokine in suppression of injurious immune responses (Rutella S, Lemoli R M. Immunol Lett 2004; 94:11-26; Conti P, et al. Immunol Lett 2003; 86:123-29; Kitani A, et al. Transforming growth factor (TGF)-b1 producing regulatory T-cells induce Smad-mediated interleukin 10 secretion that facilitates coordinated immunoregulatory activity and amelioration of TGF-β1-mediated fibrosis. J Exp Med 2003; 198:1179-1188).

Further evidence of dysfunctional regulation among the CD4+CD28null cell subpopulation is indicated by their relatively diminished expression of Treg marker, FoxP3 (Rutella S, Lemoli R M. Immunol Lett 2004; 94:11-26), which, to the best of our knowledge, is another novel finding of the present study (FIG. 1D). CD28 signaling has been implicated in the generation of CD4+CD25+Tregs (Tai X, Cowan M, et al. CD28 costimulation of developing thymocytes induces FoxP3 expression and regulatory T cell differentiation independently of interleukin 2. Nat Immunol 2005; 6: 152-56), and lack of this particular costimulatory function could possibly account for the comparative paucity of FoxP3 induction in the CD4+CD28null cells. The net effects of these altered regulatory processes could result in relatively imbalanced (and, thus, more proinflammatory and injurious) responses to engagements with antigens (including alloantigens) by T-lymphocyte populations that have increased proportions of CD4+CD28null cells.

Unlike reports based on studies of CD4+CD28null cell lines derived by extensive propagation ex vivo after initial procurements from patients with autoimmune conditions (Vallejo A N, et al. Trends Mol Med 2004; 10:119-24; Vallejo A N. Rev 2005; 205:158-69; van Leeuwen E M M, et al. J Immunol 2004; 173:1834-41; Goronzy J J, et al. Arthritis Rheum 2004; 50:43-54; Raffeiner B, et al. Arthritis Res Ther 2005; 7: R1312-R1320; Zal B, et al. Circulation 2004; 109:1230-35; Lamprecht P, et al. Thorax 2001; 56: 751-57; Komocsi A, et al. Am J Pathol 2002; 160:1717-24; Snyder M R, et al. J Immunol 2002; 168:3839-46; Liuzzo G, et al. Circulation 2000; 101:2883-88; Hirokawa M, et al. Bone Marrow Transplant 2001; 27:1095-1100; Schirmer M, et al. J Immunol 1998; 161:1018-25; and Pawlik A, et al. Transplant Proc 2003; 35:2902-04), the freshly isolated CD4+CD28null cells from recipients are clearly able to proliferate (FIGS. 4A-4C). It is unlikely that this seeming discrepancy is attributable to a fundamental biologic difference, or an epiphenomena of the immunosuppressant environment in which these cells arose in the transplantation recipients, because essentially similar results are seen among CD4+CD28null cells freshly isolated from patients who have not undergone transplantation (and are medication free) having other immunologic lung diseases. Instead, it seems far more likely that the replicative senescence of CD4+CD28null cell lines described in previous reports is a consequence of their protracted in vitro propagation. The proportion of CD4+CD28null among transplant recipients with BOS is comparatively large (a possible effect of the intense antigenicity of alloantigens, see Krensky A M, et al. N Engl J Med 1990; 322:510-17), enabling procurement of adequate numbers of freshly isolated cells for studies, thereby avoiding potential introduction of functional changes induced by repeated in vitro propagations.

The finding that a T-cell subpopulation with unusual pathogenic potential is comparatively resistant to a class of medications that is a mainstay of transplantation could also have practical relevance (FIGS. 1D, 3, 4B, 4C. A clinically frustrating and perplexing problem of allograft transplantation centers on the mechanism by which allograft injuries continue to occur and progress despite treatment with intense immunosuppression (Boehler A, Estenne M. Eur Respir J 2003; 22:1007-18; Trulock E P, et al. J Heart Lung Transplant 2006; 25:880-92; Estenne M, Hertz M I. Am J Respir Crit. Care Med 2002; 166:440-44). If a disease-causing T-cell subpopulation developed relative drug resistance, a consequence perhaps of phenotypic alterations induced by repetitive antigen-driven proliferations, it could be expected that chronic therapy with the agent(s) would result in considerable selection pressure for accumulation of the resistant cell subpopulation(s), and a likely predilection for treatment failures.

Moreover, these drug treatments could exert other deleterious effects if the more susceptible T cells also had useful specificities (e.g., avidities for microbial antigens), and increased frequencies of infections are seen in transplant recipients with CR (Boehler A, Estenne M. Eur Respir J 2003; 22:1007-18; Trulock E P, et al. J Heart Lung Transplant 2006; 25:880-92; Estenne M, Hertz M I. Am J Respir Crit. Care Med 2002; 166:440-44). Alternatively, or in addition, the more susceptible T cells could be rejection-inhibiting regulatory T cells, possibly including IL-10-producing regulatory T cell, type 1 and/or CD4+CD25+FoxP3+ Treg (Conti P, et al. Immunol Lett 2003; 86:123-29 cited in full earlier), and globally decreased frequencies of putative Treg cells have been reported in recipients with BOS (Bharat A, et al. Induction of IL-10 suppressors in lung transplant patients by CD4+CD25+ regulatory T cells through CTLA-4 signaling. J Immunol 2006; 177:5631-38). In addition to specific antigen-driven proliferations, expansions of the residual drug resistant (and potentially pathogenic) lymphocytes could also be enhanced by their homeostatic proliferations in the periphery to fill the “void” created by proliferative blockade of other (more susceptible) T-cell populations (Wu Z, et al. Homeostatic proliferation is a barrier to transplantation tolerance. Nat Med 2004; 10: 87-92). Without wishing to be limited by theory, this hypothesis may be further supported by demonstrations that drug resistant lymphocyte clones may emerge in autoimmune diseases (Jendro M C, et al. Emergence of oligoclonal T cell populations following therapeutic T cell depletion in rheumatoid arthritis. Arthritis Rheum 1995; 38: 1242-51), and the extent of T-cell oligoclonality in transplant recipients with CR can become extreme (analogous to T-cell leukemia) in the presence of chronic immunosuppression and multiple-interval treatments (Duncan S R, et al. Am J Respir Crit. Care Med 2002; 165:1439-44); Duncan S R, et al. T-cell clonal proliferations in lung transplant recipients with obliterative bronchiolitis. J Clin Invest 1996; 97:2642-50).

The present findings also raise several questions and identify potential areas for additional study. Demonstrations that CD4+CD28null have specific avidities to alloantigens would compellingly implicate their role in immunologic allograft recognition and rejection, and functional assays that can directly ascertain the relative Treg activity (if any) of these cells are also amenable to experimentation. The mechanism of drug resistance in the CD4+CD28null lymphocytes is not yet known. CD8 T cells also down-regulate CD28 with chronic antigen stimulations and this process too could have relevance for lung transplantation, but only initial characterizations of these particular cells have so far been performed.

This study was focused on investigating immunopathogenic phenomena per se, and, as such, any recipient with potentially confounding recent infections, changes in immunosuppression, or known ACR was excluded from recruitment. Thus, these data may have incorporated cryptic ascertainment biases that could confound use of the peripheral CD4 T cell measures in actual patient management. Furthermore, subject recruitment here was limited to recipients seen in outpatient clinics, and it is possible that analyses of more gravely ill, hospitalized patients could have other, poorly defined confounders. Preliminary results accrued to date in an ongoing prospective, longitudinal Cohort trial, however, suggest that acute bacterial infections do not alter CD28% in lung allograft recipients, and concurrent (albeit occult) ACR did not seemingly affect these measures in either the present study (Table 2), nor in a prior investigation of renal allograft recipients (van Leeuwen E M M, et al. J Immunol 2004; 173:1834-41).

The present findings do suggest that characterizations of circulating CD4 T cells can be useful as a means of predicting impending deterioration of allograft function in individual lung transplant recipients. If so, and given the apparent correlation between interval changes of CD28 expression with corresponding lung function (FIG. 8A), it may also be likely that serial CD28 determinations in individual recipients could have greater diagnostic and prognostic accuracies than the random cross-sectional measurements that were performed in this study.

In summary, the present data show that the extent of CD28 expression on circulating CD4 T cells among lung transplant recipients is associated with later allograft function, and it seems probable that similar considerations may apply to recipients of other allogeneic organ transplantations (Pawlik A, et al. Transplant Proc 2003; 35:2902-04). Heightened surveillance and/or interventions could be directed to those recipients at greatest risk, while possibly obviating toxic treatments among those destined for more indolent courses.

### 7. EXAMPLE

### CD25 as a Biomarker

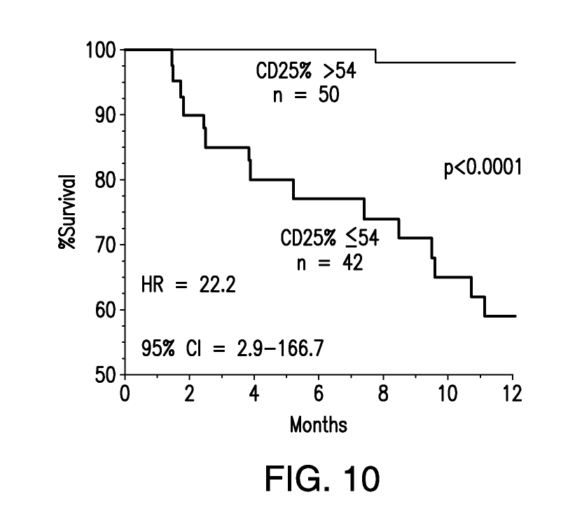

FIG. 10 shows dichotomization of IPF patients based on the percentages of their circulating CD4 T-cells that co-express CD25 (greater than 54% versus less than or equal to 54%, with high CD25 correlating with longer survival), showing that CD25 is a very specific biomarker for survival. CD25 is incrementally down-regulated on CD4 T-cells that have undergone successive replication cycles and is easily quantitated by flow cytometry. The sensitivity of this assay is much better than of CD28% alone.

### 8. EXAMPLE

### CD4 as a Biomarker

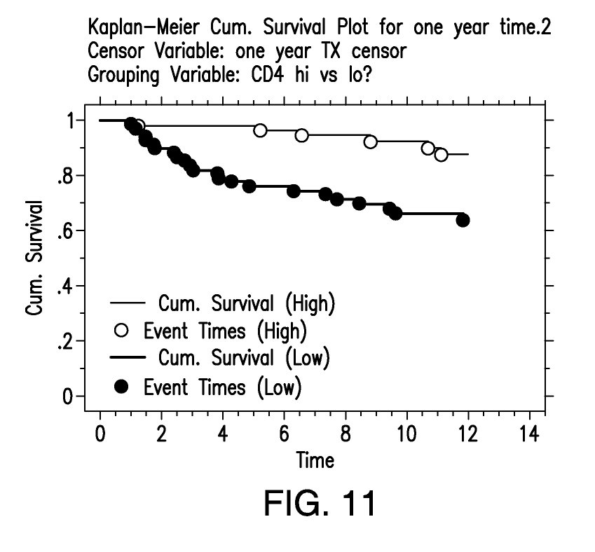

FIG. 11 shows that CD4 is a specific biomarker for survival of IPF patients For 66 patients having a CD4 population of greater than 31.3% of total PBMNC88 percent survived for at least one year. In contrast, for 72 patients having a CD4 population less than or equal to 31.3%, only 64 percent survived after one year.

### 9. EXAMPLE

### CD8+28+ as a Biomarker

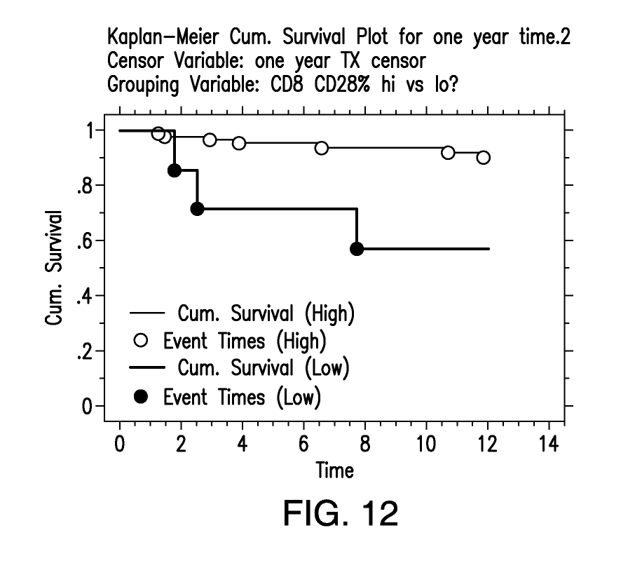

FIG. 12 shows that cells expressing both CD8 and CD28 (“CD8+28+”) are a specific biomarker for survival of IPF patients. For 84 patients having a CD8+28+ population of greater than 21% (of CD8+ cells), 90 percent survived for at least one year. In contrast, for 8 patients having a CD8+28+ population less than or equal to 21%, only 57 percent survived after one year.

### 10. EXAMPLE

### CD45RO as a Biomarker

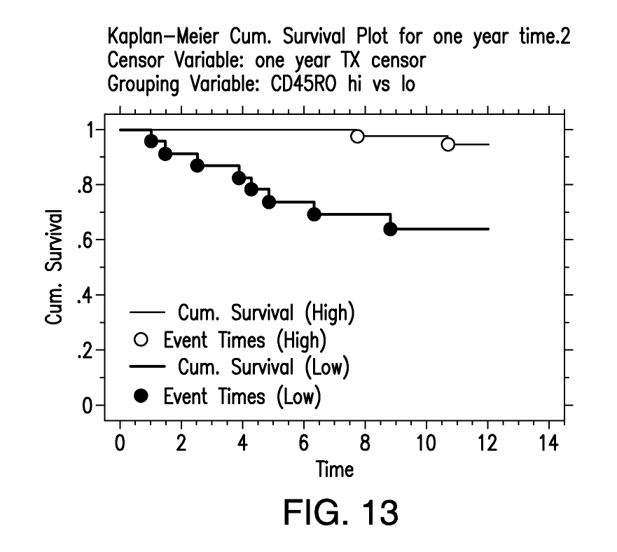

FIG. 13 shows that cells expressing both CD4 and CD45RO (“CD4+45R0+”) are a specific biomarker for survival of IPF patients. For 50 patients having a CD45RO+/total CD4+ (the proportion of all CD4 that express CD45RO) population of greater than 87.1%, 95 percent survived for at least one year. In contrast, for 25 patients having a CD4+45+RO population less than or equal to 87.1%, only 57 percent survived after one year.

### 11. EXAMPLE

### CD4+DR as a Biomarker

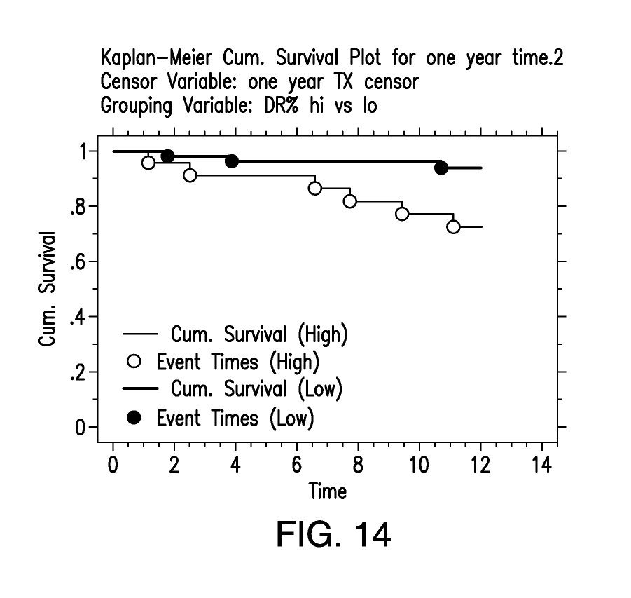

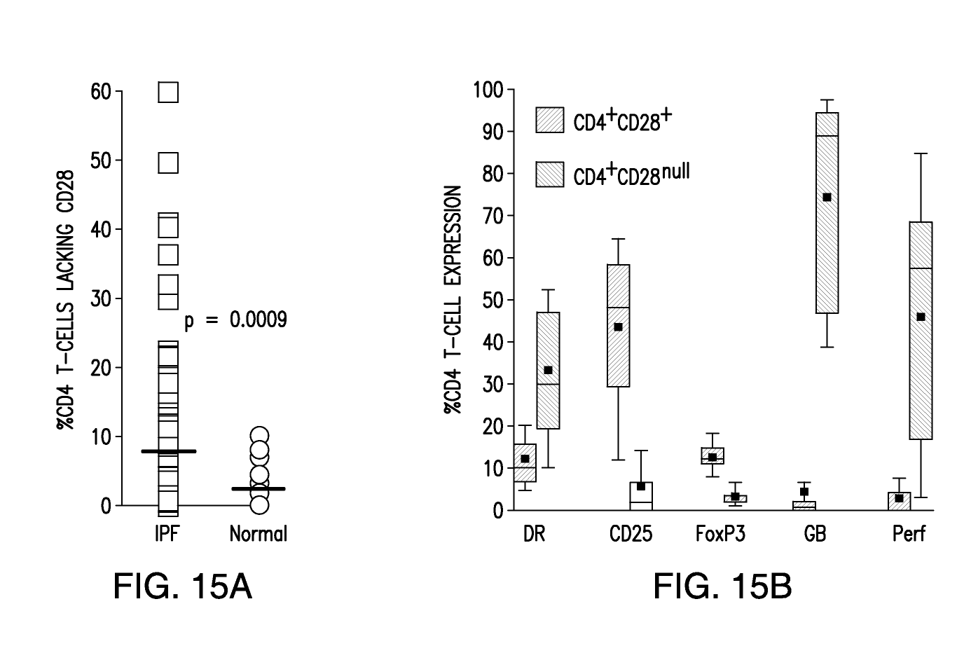

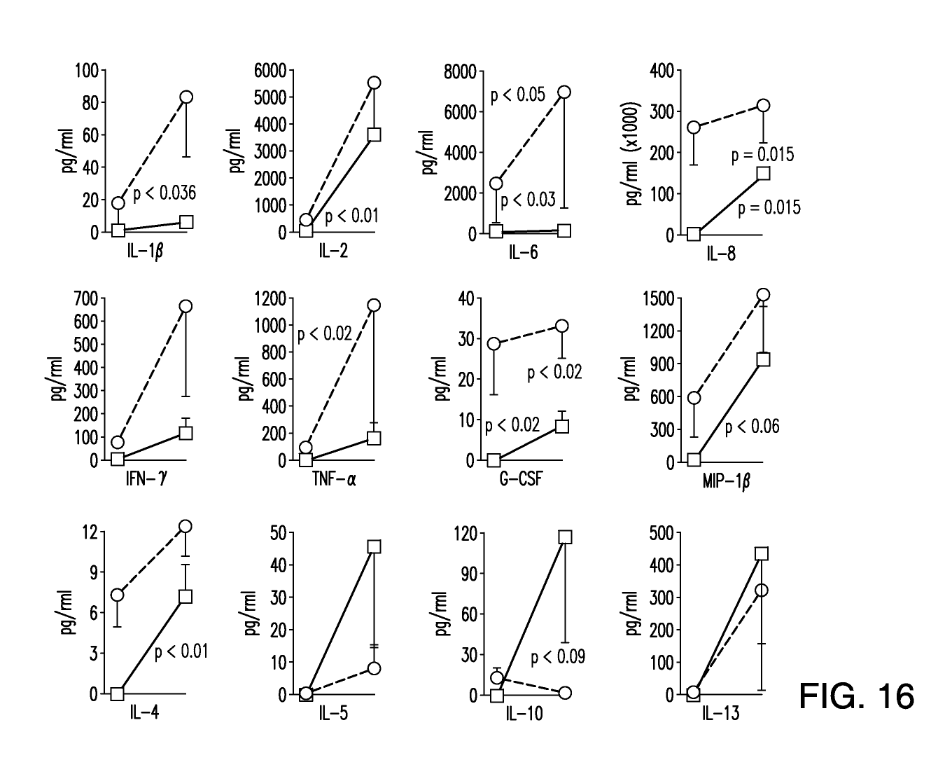

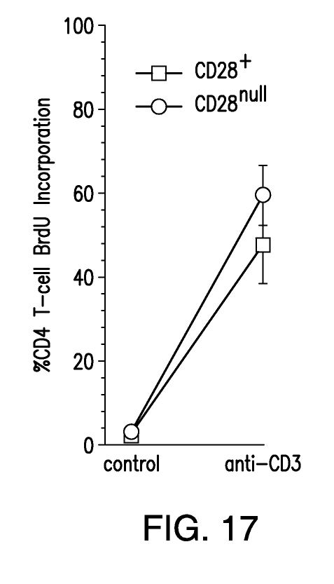

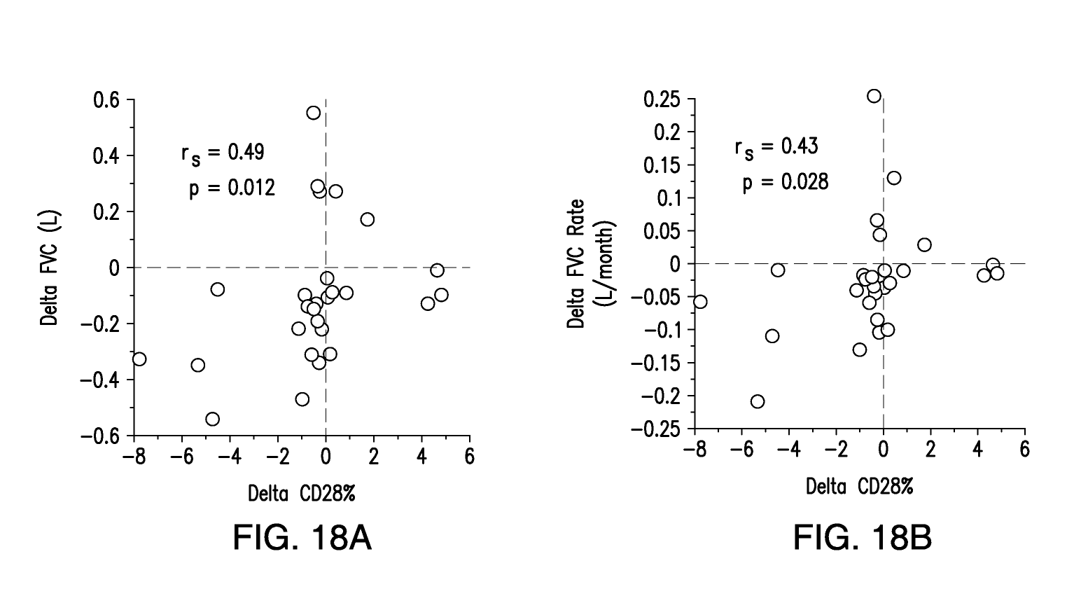

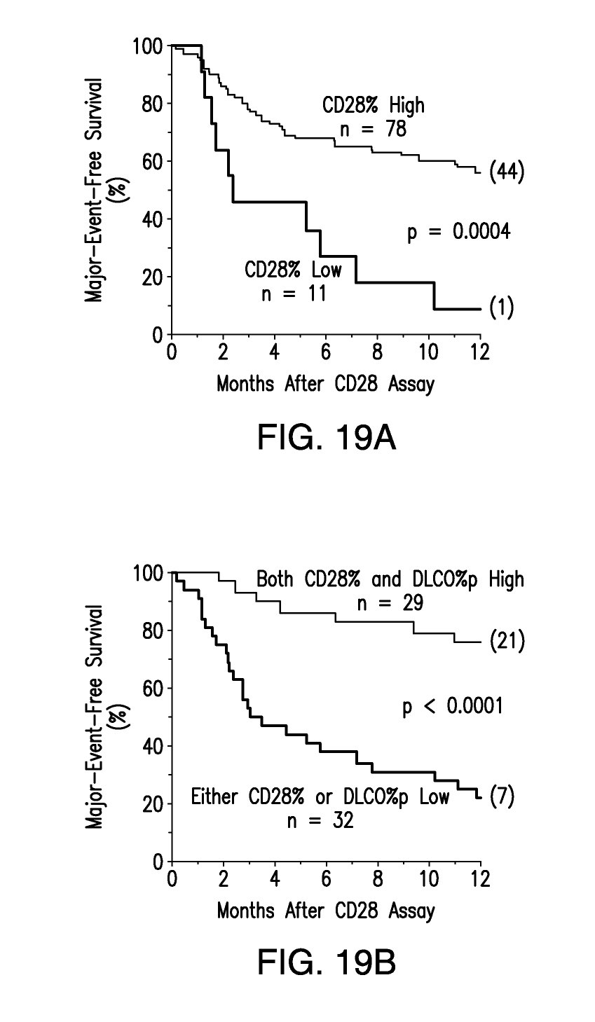

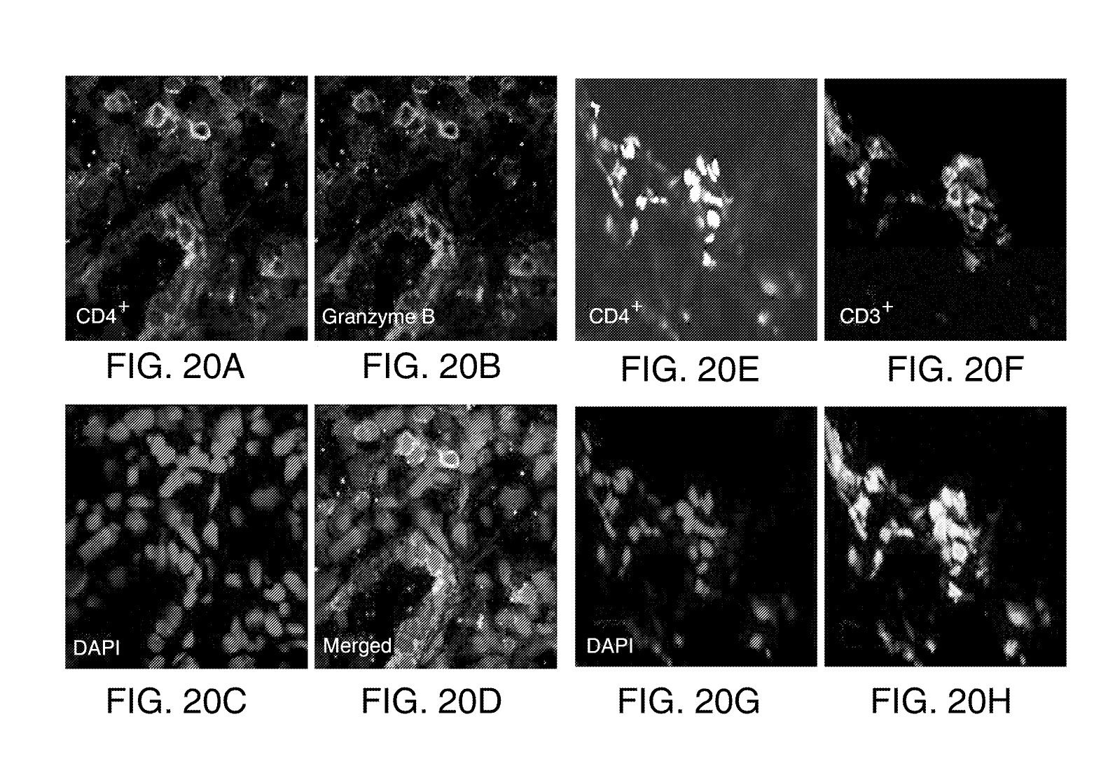

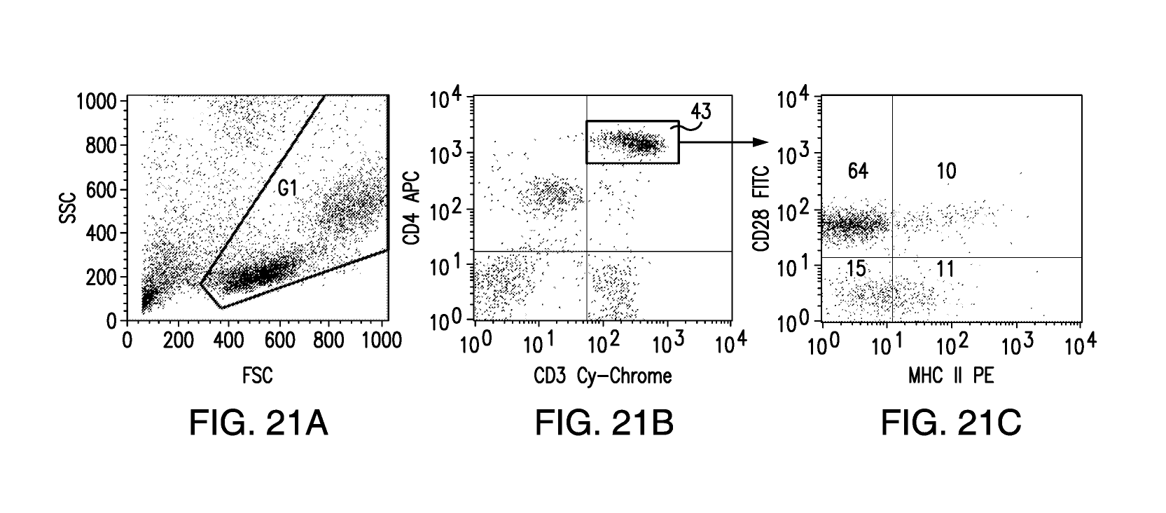

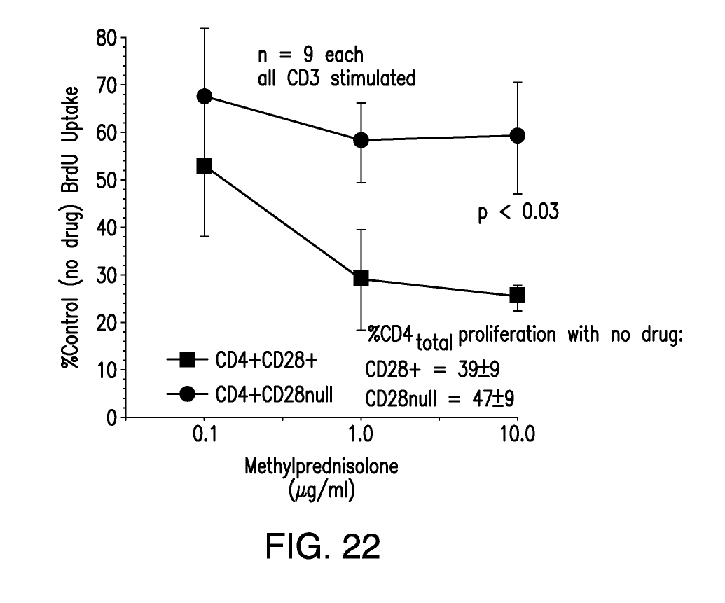

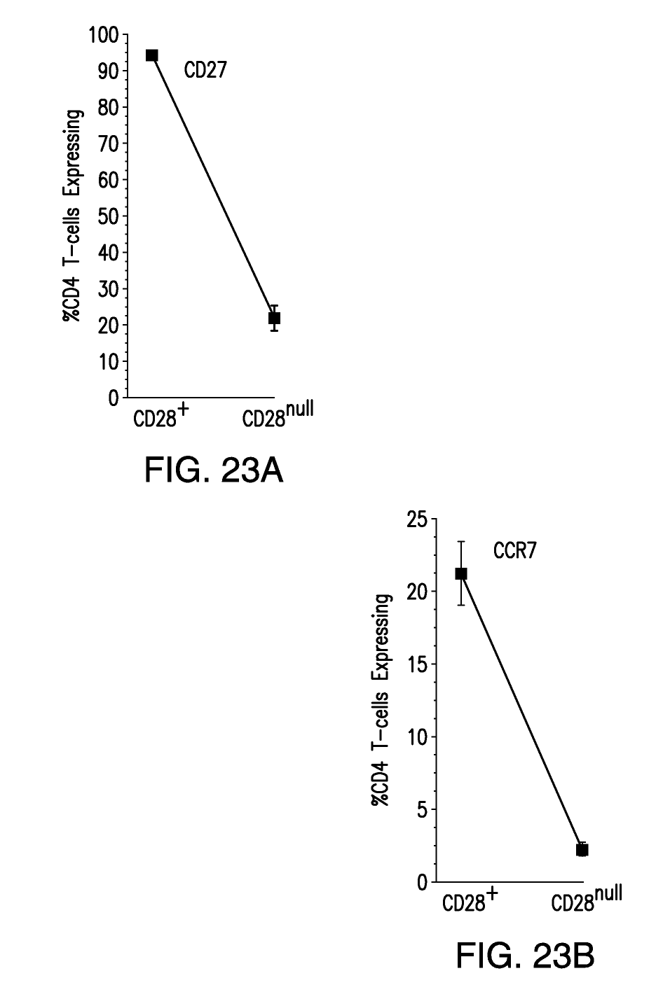

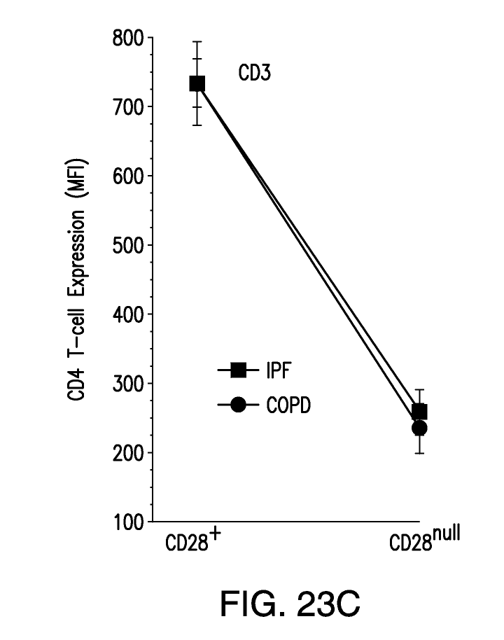

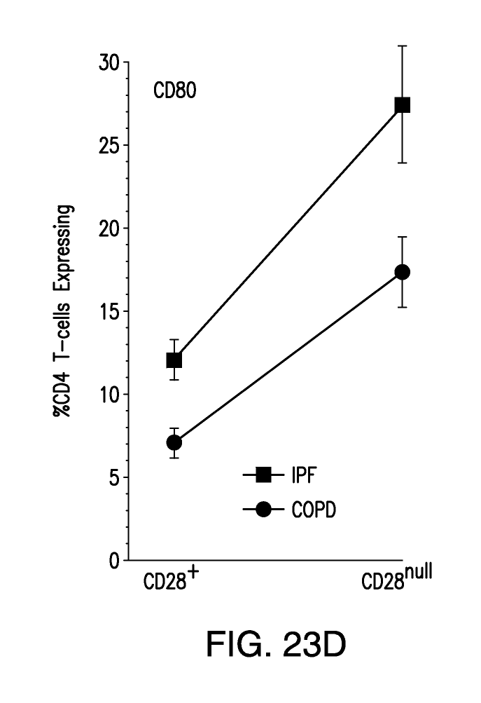

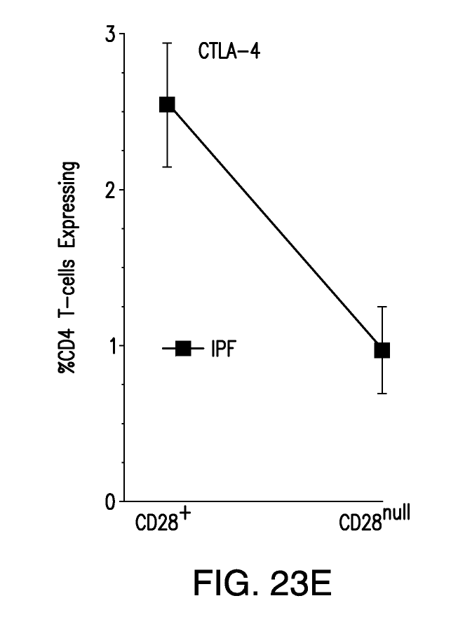

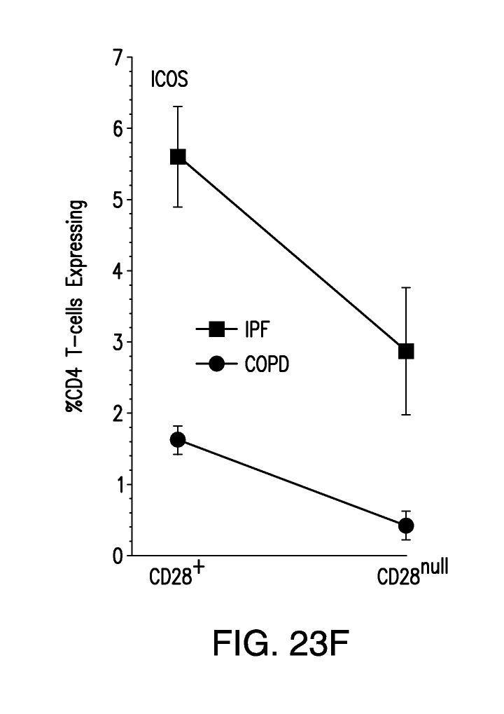

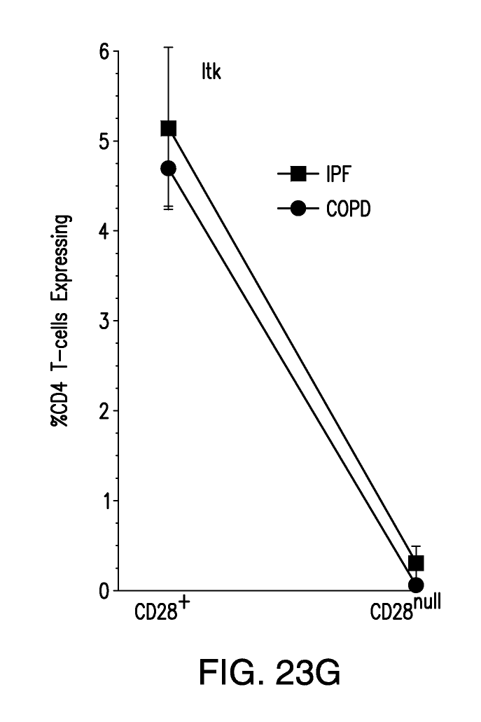

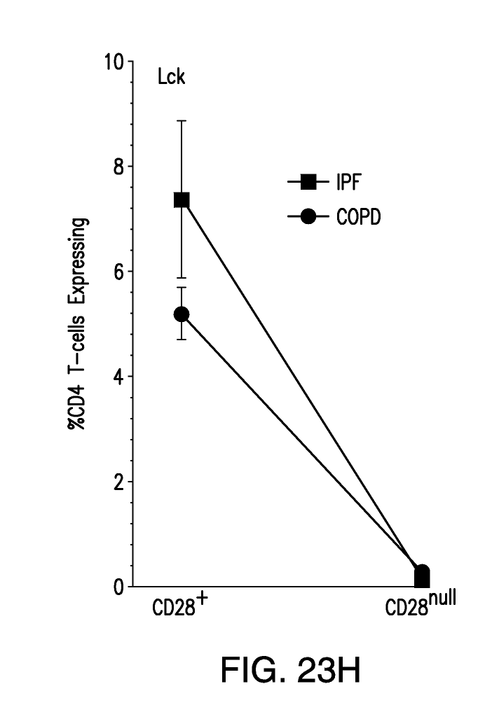

FIG. 14 shows that cells expressing both CD4 and DR (“CD4DR”) are a specific biomarker for survival of IPF. For 23 patients having a CD4DR population of greater than 87.1% of CD4+ cells, 73 percent survived for at least one year. In contrast, for 60 patients having a CD4DR population less than or equal to 87.1%, 94 percent survived after one year.

### 12. EXAMPLE

### CD27 and CCR7 as Biomarkers

As shown in FIGS. 23A-H, in IPF patients, many phenotypic characteristics are highly altered among the daughter progeny of those T-cells that have undergone repeated, antigen-stimulated cell division (here defined as CD28null, with lesser stimulated or unstimulated T-cells denoted as CD28+). Here, expression of CD27 and CCR7 are shown to be reduced in CD4+CD28null cells (FIGS. 23A and B, respectively; subject numbers here are >50 for each data point and paired comparisons of all are highly significant (r<<0.0001)). FIGS. 23C-H show that CD4+CD28null cells express less CD3, more CD80, less CTLA-4, less ICOS, less Itk, and less Lck, than CD4+CD28+ cells.

### 13. EXAMPLE

### CD28 Down-Regulation on Circulating CD4 T-Cells is Associated with Poor Prognoses of Patients with Idiopathic Pulmonary Fibrosis

### 13.1 Materials and Methods

Subjects, Assignments of Diagnoses, and Treatment Decisions. IPF patients were recruited from referrals to the interstitial lung disease (ILD) clinic at the University of Pittsburgh. Normal controls were recruited by solicitation, and did not have any known systemic or immunologic disorders that affect CD28 expression [19]. All subjects gave written informed consent per the University of Pittsburgh Investigational Review Board (IRB). IPF diagnoses were prospectively established by expert, specialized ILD clinicians who analyzed all available information, including medical histories and physical exams, PFTs, laboratory studies that included serologic tests for autoimmune syndromes, chest radiographs and computerized tomography scans, and histologic evaluations of lung biopsies and pulmonary explants surgically removed during transplantations. All IPF subjects who provided blood or tissue specimens here fulfilled consensus diagnostic criteria [1]. None had ongoing acute exacerbations of IPF [3], clinical or serologic evidence of autoimmune disorders, malignancies, or concurrent infections. Subjects were also excluded if they had histologic patterns on lung biopsy (or explant) other than usual interstitial pneumonia (UIP) or, in one case, end-stage fibrotic lung disease with honeycombing [51]. The clinicians caring for these patients were blinded to the results of these experimental T-cell studies. Conversely, the T-cell assay results were analyzed by laboratory-based investigators blinded to subject identities, disease manifestations, and clinical outcomes.

Cell Preparations and Flow Cytometry.

Mononuclear cells were isolated from peripheral blood of study subjects by density gradient centrifugation for use in flow cytometry or functional assays. PBMNC for cell surface characterizations were incubated with monoclonal antibodies (MAb) immediately after their isolation, and then promptly analyzed by flow cytometry. Unless otherwise denoted, MAb, including isotype control antibodies, were purchased from BD Pharmingen (San Diego, Calif.). These MAb included CD4 (RPA-T4), CD28 (CD28.2), CD8 (HIT8a), MHC Class II (DR) (L243), CD25 (M-A251), granzyme B (GB11), and perform (3G9). Anti-FoxP3 MAb (FJK-16S) was purchased from eBioscience, San Diego, Calif. Quantitations were performed on ≧10,000 live cells and analyzed using a BD FACSCalibur (BD Bioscience, San Jose, Calif.). This instrument has 488 nm and 635 nm lasers, and employs long pass, short pass, and bandwith filters that transmit wavelengths 530/30 nm (FL1), 585/42 nm (FL2), 670 nm (FL3), and 661/16 nm (FL4). Flow cytometry gates were set using control fluorochrome positive and negative PBMNC (including isotype controls). CD28 and other phenotypic markers were quantitated within respective CD4+CD28+ and CD4+CD28null cell subpopulations, as detailed elsewhere [20]. In brief, PBMNC were stained with anti-human CD4-allophycocyanin (APC) and anti-human CD28-fluorescein isothiocyanate (FITC) MAb, and individual aliquots of these were also stained with phycoerythrin (PE)-conjugated MAb against other cell surface epitopes of interest (e.g., HLA-DR, CD25, etc.). CD4bright cells corresponding to the CD4 T-cell subpopulation were selectively gated (from within the live cell gate) for further characterizations (FIGS. 21A, 21B) [20], [52]. CD28 and other phenotypic markers were quantitated within respective CD4+CD28+ and CD4+CD28null subpopulations (FIG. 21C). Intracellular mediators (perforin, granzyme B, and FoxP3) were also quantitated among freshly isolated PBMNC aliquots that had been identically stained with anti-human CD4 and CD28 MAb, and then fixed and permeabilized prior to incubation with MAb having specificities for the intracellular molecules, per manufacturers' protocols. The CD28% value for individual patients was defined as the proportion of their circulating CD4 T-cells that co-express CD28 [20]. Based on prior observations, it was reasoned that study of CD4 T-cell phenotypic markers (e.g., MHC Class II, CD25, etc.) in 24 IPF subjects would allow detection of intergroup differences, provided that their cellular characteristics resembled those of other patient populations [20]. Specimens for the T-cell phenotype studies were selected randomly, without prior knowledge of the corresponding CD28% or subject's clinical characteristics.

Cytokine Assays.

CD4 T-cells cells for cytokine and proliferative assays were isolated from PBMNC by negative selection with immunomagnetic beads (Miltenyi Biotech, Auburn, Calif.). CD28null cells and CD28+ cells were segregated from among this CD4 T-cell population by staining with anti-CD28-phycoerythrin (PE), followed by positive selection of the latter with anti-PE immunomagnetic beads (Miltenyi Biotech) [20]. 1×10⁵ CD4 T-cells (autologous CD28+ and CD28null) were separately cultured for two days in 96 well plates in both stimulated (10 μg/ml plate-bound anti-CD3 MAb [OKT3]) and un-stimulated (basal) conditions. Culture supernatants were analyzed for cytokine productions using a Bio-Plex™ protein suspended bead array platform (Bio-Rad, Hercules, Calif.), following the manufacturer's protocols. Prior observations indicated that study of six (6), randomly-selected specimens would enable detections of intergroup cytokine production differences, if characteristics of the IPF CD4 T-cell subpopulations were similar to those seen with cells isolated from another patient group [20].

CD4 T-Cell Proliferation Assays.

These methods have been described elsewhere [20]. In brief, segregated autologous CD28+ and CD28null CD4 T-cell subpopulations, isolated by immunomagnetic beads, were individually cultured in duplicate conditions, including unmanipulated (control) incubations, and in wells previously coated with anti-CD3 MAb (see above). Autologous nurse cells, positively selected from PBMNC during the initial CD4 T-cell isolations, were added in a 3:1 nurse cell:CD4 T-cell ratio. All CD4+ cells were depleted from the nurse cells, prior to their use in cultures (<<1% residual), by positive selection with anti-CD4-coated magnetic beads (Invitrogen, Carlsbad, Calif.). Proliferation within the respective CD4 subpopulations were measured by bromodeoxyuridine (BrdU) incorporation, using reagents and methods supplied in a kit (BD Pharmingen). BrdU in a final concentration of 10 uM was added to cultures 2 days prior to their harvests. After 5±1 days of culture, cells were harvested from wells and stained with anti-CD4 MAb, fixed and permeabilized, and then incubated with anti-BrdU and 7-amino-actinomycin (7-AAD). The proportion of proliferating CD4+ T-cells (BrdU+) among the viable cells (≧diploid DNA content) was determined by flow cytometry.

Lung Immunostaining.

Explanted lung tissues from two IPF patients who underwent pulmonary transplantations were obtained from the University of Pittsburgh Health Sciences Tissue Bank. These tissues had been embedded in optimal cutting temperature (OCT) media immediately after explantation and stored frozen at −80° C. Lung tissues were cut into 4 micron thickness using a cryostat, and placed on slides kept at −80° C. until used. These frozen sections were fixed using 2% paraformaldehyde for 20 minutes. Cell permeabilisation was carried out by using 0.1% Triton X-100 in phosphate buffered saline (PBS) for 15 minutes. Sections were rehydrated by three cycles of incubation for five minutes each in PBS, followed by washing with 0.5% bovine serum albumin (BSA) in PBS. The slides were then blocked with 5% donkey serum (Sigma-Aldrich, St. Louis, Mo.) in PBS containing 3% BSA for 45 minutes. Slides were incubated overnight at 4° C. in blocking solution supplemented with rabbit polyclonal anti-human CD3 (ab5690, Abeam, Cambridge, Mass.), and mouse anti-human CD4 MAb (SK3, BD Biosciences). Separate sections were similarly prepared with rabbit polyclonal anti-human granzyme B (ab4059, Abeam) and SK3 MAb. Sections were washed 5 times with 0.5% BSA in PBS to remove unbound primary antibodies, and then incubated with Alexia 568-conjugated goat anti-rabbit and Alexia 488-conjugated goat anti-mouse secondary antibodies (both from Invitrogen) for one hour at 28° C. All primary and secondary antibodies were used at 1:100 dilutions. Subsequently, the slides were washed five times with 0.5% BSA in PBS, followed by five washes with PBS alone, with each wash lasting for five minutes. Cell nuclei were counterstained with 4′,6-diamidino-2-phenylindole (DAPI) (Vector Lab, Burlingame, Calif.) for one minute and briefly washed two times in PBS. Slides were then cleaned and cover slips were mounted using Gelvatol media obtained from the Center of Biological Imaging at the University of Pittsburgh. The slides were dried overnight at 4° C. while protected from light, and images were visualized using an Fluorview 1000™ confocal microscope (Olympus, Center Valley, Pa.). Digital image data were archived and prepared for publication using Adobe Photoshop software (Adobe Systems Inc., Mountain View, Calif.).

Statistical Analyses.

Intergroup comparisons of continuous data were analyzed by Mann-Whitney rank-sum tests. Dichotomous variables were analyzed by Chi-square. Correlations between continuous variables were established by Spearman rank order. Survival analyses were performed using product-limit estimation, with comparisons by Log-rank (Mantel-Cox). Odds ratios and confidence intervals were established by logistic regression. Data were analyzed using StatView™ (SAS, Cary, N.C.) Receiver operator characteristic (ROC) analyses were examined using Analyse-it™ v 2.12 (Analyse-it Software, Ltd, Leeds, UK). Significance was defined as p<0.05. Data are depicted as mean±SEM.

Results

CD4 T-Cell Expression of CD28 among IPF Patients. Eighty-nine (89) IPF subjects had one or more assays of their peripheral blood CD4 T-cells. Unless otherwise specified, only results of their latest determinations are described. Clinical observation periods following their last experimental T-cell assays exceeded 12 months in all subjects who survived and/or did not have lung transplantation. The proportion of total circulating CD4 T-cells that co-expressed CD28 were determined for each subject, and these values are defined here as CD28% [20]. CD28 down-regulation was striking in some IPF patients (FIG. 15A), and nineteen (21%) of these subjects had CD28%<90. Ages of the 32 normal, healthy controls (67.9±1.1 years old) were comparable to those of the IPF subjects (p═N.S.), None of the normal controls had CD28%<90, and their aggregate values of this parameter (97.9±0.4) were significantly greater than those of the IPF subjects (FIG. 15A).

Phenotypic Features of IPF CD4+CD28null Cells.

A series of assays were performed (FIG. 15B) to ascertain if the CD4+CD28null cells of IPF patients exhibited unusual phenotype characteristics, as has been observed elsewhere in comparable cells from patients with chronic adaptive immune disorders [19]-[34]. Because the CD4+CD28null lymphocytes are daughter progeny of repetitive, antigen-driven T-cell replication cycles [19], [20], [25], [27], [30], [31], it was hypothesized that these cells would be highly activated. A greater proportion of the IPF CD4+CD28null did express major histocompatibility antigen (MHC) Class II (HLA-DR) than the corresponding autologous CD4+CD28+ cells. However, IPF CD4+CD28null cells paradoxically expressed less CD25 than autologous CD4+CD28+ cells, and this discordant expression of activation markers has also been previously reported in cells derived from another patient population [20]. There was no apparent correlation between CD4 values (as percentages of total peripheral blood mononuclear cells [PBMNC]) and CD28% (rs=0.16, p=N.S.)

Selected intracellular factors and mediators with potential clinical relevance were also evaluated here. The lesser expression of CD25 by IPF CD4+CD28null cells prompted measurement of FoxP3, and this transcription factor and marker for regulatory T-cell (Treg) function was present in significantly fewer of these cells than among corresponding CD4+CD28+ lymphocytes (FIG. 15B).

Because pulmonary epithelial cell death may be an important pathologic process in IPF [35], potential cytotoxic functions of the CD4 T-cell subpopulations were evaluated. Cell surface expression of FasL was at or below the detection threshold for flow cytometry in both CD4 subpopulations, but the IPF CD4+CD28null lymphocytes frequently produced perform and granzyme B, in striking contrast to autologous CD4+CD28+ cells (FIG. 15B).

CD4+CD28null T-Cells of IPF Patients Produce Pro-Inflammatory Cytokines.

CD4+CD28null T-cells from patients with autoimmune diseases or other chronic immunologic disorders have abnormal productions of various cytokines that may have potential importance in disease pathogenesis [19], [20], [24]. Differential cytokine productions of autologous CD4+CD28+ and CD4+CD28null cells from these IPF patients were also evident in multiplex analyses (FIG. 16), and the mediator elaboration profiles of these lymphocytes were similar to those of comparable cell subpopulations isolated from transplantation recipients [20]. CD4+CD28null cells generally produced much greater amounts of pro-inflammatory and TH1 mediators under basal conditions, and in many cases these productions were strikingly increased by T-cell antigen receptor (TCR) cross-linking with anti-CD3 antibody (thereby replicating effects of T-cell-antigen engagements) (FIG. 16). Conversely, and with the exception of IL-4, productions of TH2 cytokines, notably including putatively immunosuppressive IL-10, were reduced among the CD4+CD28null cells in comparisons to autologous CD4+CD28+ cells. As shown in FIG. 22, CD4+CD28null cells were also observed to be relatively resistant to effects of glucocorticoids, compared to “normal” CD4 T-cells isolated from the same patients.

CD4+CD28null Lymphocytes Isolated from IPF Patients Are Capable of Further Proliferation.

Several studies of CD4+CD28null cell lines derived by extensive ex vivo propagation suggest these lymphocytes are replicatively sencescent [19]. Nevertheless, it has previously been shown that freshly isolated CD4+CD28null lymphocytes from lung transplant recipients are able to proliferate with TCR cross-linking [20], raising the possibility that the reported inability of CD4+CD28null cell lines to divide is a consequence of their protracted in vitro propagation. In order to further evaluate this hypothesis, proliferations were measured of segregated CD4+CD28+ and CD4+CD28null lymphocytes from IPF subjects after stimulation that mimics T-cell engagement with antigen-MHC complexes (e.g., plate bound anti-CD3 monoclonal antibodies). As was seen in previous study of cells isolated from allograft recipients [20], proliferation of CD4+CD28null T-cells from the IPF patients was near identical to that of autologous CD4+CD28+ cells (FIG. 17).

CD28 Expression Is Associated with Changes of Pulmonary Function in IPF Patients.

Pulmonary function tests (PFT) were ordered by physicians caring for these patients on the basis of clinical indications, while blinded to the experimental immunologic assays. Seventy-one (71) subjects had CD4 T-cell assays concurrent with measures of forced vital capacity (FVC), and 57 of these also had determinations of diffusing capacity for carbon monoxide (DLCO). CD2% values modestly, but significantly correlated with concordant DLCO of the subjects (rs=0.34, p=0.01). The cross-sectional relationships between CD28% values and other PFT parameters were not significant.

Although single, essentially random measures of CD28% had only limited correlation with concurrent PFT values, these particular T-cell assays have been shown elsewhere to be predictive for subsequent changes of physiologic measures [20]. Too many of the subjects here succumbed or had lung transplantations soon after their last experimental T-cell assays, without having had additional, interval PFTs, to render these analyses meaningful. However, 27 study subjects had prior CD28% determinations, along with concurrent PFTs, as participants of an ongoing longitudinal study.

The net change of aggregate CD28% between the preceding and latest determinations among this subpopulation was −0.44±0.53. Ten (10) of these patients had increases of CD28% (1.7±0.6) with repeat testing 4.6±0.6 months later, in contrast to the other 17 IPF subjects with interval CD28% decrements (−1.7±0.6) over 4.6±0.5 months. The FVC declined significantly less among those subjects with interval CD28% increases (−0.04±0.05 L) compared to those with decreasing CD28% values (−0.15±0.07 L) (p=0.05). Moreover, interval changes of CD28% among individual subjects were also correlated with corresponding changes of their lung volumes, in terms of both absolute measures (FIG. 18A), and as time-dependent rates (FIG. 18B). Although there were also similar trends for greater decrements of DLCO and DLCO % p among the subpopulation with interval decreases of CD28%, these intergroup comparisons did not reach statistical significance.

CD28 Down-Regulation in IPF Patients Is Associated with Clinical Outcomes.

Given that CD28% values have also been shown to associate with outcome measures in other populations with chronic immunologic disorders [20]-[23], [32]-[34], the hypothesis that these quantitative CD4 T-cell phenotype assays might have clinical prognostic value for patients with IPF was tested.

The eventual progression of lung dysfunction in IPF patients almost inevitably results in either lung transplantation or the death of those afflicted [1]. However, these dichotomous outcomes are not truly independent events. Lung transplantation ostensibly, albeit by no means invariably, diminishes or delays deaths of IPF patients, although the procedure itself is also associated with a finite inherent mortality [36]. Hence, we initially tallied occurrences of either lung transplantations or deaths as equivalent outcome end-points. Receiver operating characteristic (ROC) curve analyses indicated that cut-off values for CD28%=82 seemed to provide the best compromise of optimal specificity (the most important consideration) and sensitivity for these major, adverse clinical events.

Those IPF patients having the most extreme CD28 down-regulation, with CD28%<82 (henceforth denoted as CD28% Low), had significantly worse 12-month outcomes than the cohort with CD28%≧82 (CD28% High) (FIG. 19A). These intergroup survival differences were also apparent with six-month analyses (27% vs. 68%, CD28% Low and CD28% High, respectively, p=0.004). Deaths accounted for 24 of the major adverse events, and 20 other subjects had lung transplantations. Seventeen (17) deaths in the study population were directly attributable to respiratory failure. One of the CD28% High subjects died from a myocardial infarction, and another had fatal pulmonary thromboemboli. Five (5) other deaths (four CD28% High and one CD28% Low patient), were due to unknown causes in subjects with preexistent severe pulmonary dysfunction.

To exclude cryptic confounding by potential vagaries of the transplantation selection process (despite “double blinding” between the clinicians and laboratory investigators), post hoc actuarial analysis was performed limited to those IPF subjects who did not have lung transplantations during the year after their T-cell assays. Despite lesser statistical power due to fewer subjects, the 12-month survival of the CD28% High patients (68±6%) was still significantly greater than that of the CD28% Low cohort (20±18%) (p=0.007).

CD28% High and CD28% Low cohorts were well matched with respect to demographic characteristics (Table 9). CD4+CD28null cells can reportedly accumulate with aging, but this typically occurs during the eight decade of life or later [19], [25]. Nonetheless, there was no apparent correlation between age and CD28 expression in the IPF cohort (rs=0.1, p=0.36), the overall age distributions of the comparison populations here were equivalent (Table 9), and both the number (n=15) and proportion (19%) of octogenarians among CD28% High were actually greater than among the CD28% Low (n=1 [9%]). Absolute DLCO values were decreased in the CD28% Low, but there was a less significant intergroup difference of the DLCO as a percentage of normal predicted values (DLCO % p) (Table 9).

TABLE 9

| | (CD28% High) | (CD28% Low) |
|---|---|---|
| n | 78 | 11 |
| Age (years) | 69.6 ± 1.6 | 71.7 ± 2.6 |
| Male -n (%) | 55 (71) | 8 (73) |
| Former Smoker -n (%) | 56 (72) | 8 (73) |
| Caucasian -n (%) | 76 (97) | 10 (91) |
| African-American -n (%) | 1 (1) | 1 (9) |
| Native American -n (%) | 1 (1) | 0 |
| Lung Biopsy n (%) | 48 (62) | 7 (64) |
| CD28% | 96.4 ± 0.5 | 66.2 ± 4.0* |
| FVC (L) | 2.4 ± 0.1 | 2.1 ± 0.2 |
| (n) | (62) | (9) |
| FVC % p | 60.5 ± 2.4 | 57.7 ± 4.3 |
| (n) | (62) | (9) |
| FEV₁/FVC | 0.84 ± 0.01 | 0.85 ± 0.01 |
| (n) | (62) | (9) |
| DLCO (ml CO/min/mm Hg) | 10.7 ± 0.7 | 6.3 ± 0.9** |
| (n) | (50) | (7) |
| DLCO % p | 43.6 ± 2.3 | 31.0 ± 4.7† |
| (n) | (50) | (7) |
| Medications n (%) | | |
| None | 56 (72) | 9 (82) |
| Prednisone | 6 (8) | 1 (9) |
| IFN-γ | 2 (3) | 0 |
| IFN-γ + Prednisone | 7 (9) | 0 |

In Table 9, CD28% High denotes those subjects in whom >82% of their circulating CD4 T-cells co-express CD28. CD28% Low denotes those subjects in whom <82% of their circulating CD4 T-cells express CD28. CD28% is defined here as the proportion of circulating CD4 T-cells that also express CD28. FVC=forced vital capacity. FVC % p percentage of normal predicted FVC. FEV1/FVC=the ratio between the patient's forced expiratory volume in 1 second to their FVC. DLCO=diffusing capacity for carbon monoxide. DLCO % p=percentage of normal predicted DLCO. One CD28% High biopsy showed end-stage honeycombed fibrotic lung, whereas all other histologic patterns were usual, interstitial pneumonia. *p<0.0001, **p=0.007; †p<0.045.

The Prognostic Value of CD28 Measures Is Independent of Pulmonary Function.

Further analyses was performed to ensure that the extent of CD28 expression in these subjects was an independent variable, relative to PFTs, for clinical outcome prognostications. The odds ratio (OR) for adverse clinical outcome (death or transplantation) of IPF patients with CD28%<82 was 13.0, with 95% confidence intervals (CI) 1.6-111.1. The inclusion of PFT values, as co-independent continuous variables in a logistic regression model (in addition to CD28%), did not enhance associations with adverse events, with individual OR for FVC, as a percentage of normal predicted values (FVC %p)=1.0 (CI: 1.0 to 1.1), and for DLCO % p=1.1 (CI: 1-to-1.2).

It was hypothesized that the generally reported poor associations between single, cross-sectional PFT values and prognoses of IPF patients [37] may possibly be due, at least in part, to potential confounding effects of CD28 down-regulation. Since the mortality associated with CD28 down-regulation per se appears to be independent of concurrent physiologic measures, the unrecognized inclusion of patients with significant proportions of CD4+CD28null cells in earlier analyses may have confounded assessments based solely on cross-sectional measures of pulmonary function (i.e., increase the “noise to signal ratio”). Moreover, the identification of other facile measures that are also predictive of patient outcomes, in addition to assays of circulating CD4 T-cell CD28 expressions, could be useful for clinical management, particularly if these determinations could be applicable to the IPF subjects who do not have significant CD28 down-regulation.

Accordingly, the prognostic values of PFT abnormalities in the CD28% High cohort was evaluated to assess whether this subpopulation could be further stratified for risks of adverse clinical outcomes (death or lung transplantation). ROC curve analyses indicated that values of FVC % p<52 and DLCO % p<38 had the best respective combinations of specificities (the most important consideration) and sensitivities for adverse clinical outcomes. Fifty (50) CD28% High IPF patients had complete pulmonary function tests (both FVC and DLCO measures) concurrent with their CD4 T-cell assays and were evaluable here. Twelve-month freedom from adverse outcomes of the CD28% High patients with FVC % p≧52 (n=33) was 70+8%, compared to 29+11% among those with FVC % p<52 (p=0.001). The DLCO % p seemed to provide slightly better risk stratification among the CD28% High patients, with corresponding one-year freedoms from adverse events of 76±8% and 29±10% for those with values above (n=24) and below (n=26) the cut-off of 38, respectively (p=0.0003).

The potential utility of risk assessments using combinations of independent prognostic parameters may be indicated by survival analyses using either CD28% or DLCO % p as outcome indicators. Thirty-two (32) of the IPF patients studied here had either extreme CD28% down-regulation (CD28%<82) or DLCO % p values <38. Of the 11 patients that had CD28 down-regulation (Table 1), six (6) also had DLCO % p<38, another had a DLCO % p=54, and four others did not have DLCO determinations concurrent with their CD4 assays. A total of 29 IPF patients had both CD28%≧82 and concurrent DLCO % p≧38. Twelve-month freedom from adverse clinical outcomes was 76±8% for the latter, compared to 22±7% among those patients with either CD28 down-regulation or DLCO % p<38 (FIG. 19B). The OR for the association between adverse clinical events and the presence of either CD28%<82 or DLCO % p<38 was 11.2, with 95% CI 3.4-37.1. The addition of low FVC % p criteria (FVC % p<52) to the combined low CD28% and/or low DLCO % p values slightly weakened the association with adverse events (OR: 10.3; CI: 3.0-34.9). Thus, the presence of either CD28% down-regulation or low DLCO % p values, as simultaneous, combined, cross-sectional ascertainments, would seemingly identify a large proportion of the patients within an IPF cohort who are at greatest risk for serious adverse events within the next few months.

CD4+CD28null T-Cells Are Present in IPF Lungs.

Since the IPF subjects here were scrupulously free of extrapulmonary pathology or inflammatory foci, it was hypothesized the circulating CD4+CD28null cells of these patients originated in their diseased lungs. Moreover, the presence of these unusual T-cells in IPF lungs would further imply the associations of CD28 down-regulation with disease manifestations here (FIGS. 18 and 19) may not be merely epiphenomenal. Accordingly, confocal microscopy was used to examine sections of IPF lungs removed during therapeutic pulmonary transplantations.

It was reasoned that tissue imaging assays based on positive expressions of specific CD4±CD28null cell characteristics would be more reliable and preferable to finding that CD28 did not appear to be present (which could also be due to poor antibody staining). Granzyme B seemed a useful surrogate marker for this purpose, since the majority of CD4+CD28null cells produce this cytotoxic mediator, unlike “normal” CD4+CD28+ T-cells (FIG. 15B). However, since natural killer (NK) cells can also express CD4 and produce cytotoxic mediators, it was necessary to confirm the intrapulmonary CD4+granzyme B+ cells also co-expressed CD3 (and are thus T-cells).

Confocal microscopy images revealed numerous cells positive for CD4, CD3 and granzyme B were widely distributed within alveolar epithelial areas of the IPF lungs, as well as in or proximate to fibrotic foci of both explant specimens (FIG. 20).

Discussion

These data demonstrate that the CD4+CD28null T-cells of IPF patients exhibit unusual characteristics (FIG. 15B and FIG. 16) that are very similar to analogous cells isolated from other patient populations with chronic immunologic diseases [19]-[34]. Infiltrations of these unusual CD4+CD28null T-cells were evident in IPF lungs (FIG. 20). Changes of CD4 T-cell CD28 expression with replicate studies of subjects were directly correlated with corresponding changes of their lung volumes (FIG. 18). Most importantly, marked CD28 down-regulation on the peripheral CD4 T-cells of individual IPF patients is associated with a higher likelihood of their requiring lung transplantations or dying during the next year (FIG. 19A), and these T-cell measures also seem to complement and enhance the prognostic utility of pulmonary physiologic testing (FIG. 19B).

Nearly all normal CD4 T-lymphocytes express CD28 on their cell surfaces [38], and finding significant proportions of circulating CD4+CD28null T-cells is distinctly abnormal [19]-[34]. In all cases of which we are aware, possibly excepting the very aged, CD28 down-regulation on significant proportions of peripheral CD4 T-cells is a specific indicator of an ongoing, chronic inflammatory response, as previously described in patients with autoimmune syndromes, persistent inflammation-provoking infections, or other long-standing immunologic disorders [19]-[34].

The pathophysiologic importance of the CD28 “deficiency” per se in these patients is uncertain. Cognate interactions of T-cell CD28 with ligands (e.g., CD80 and CD86) provide a “second signal” for lymphocyte activation, in conjunction with the primary signals mediated by TCR engagements with antigen-MHC complexes. Nonetheless, neither previously activated (“memory”) CD4 nor naïve CD8 T-cells require CD28 for activation, and other costimulatory molecules can also provide the second signal necessary for initial activations of naïve CD4 cells [38], [39]. Since CD28 costimulation is not invariably essential to evoke T-lymphocyte responses, the absence of this molecule on CD4 T-cells of patients with immunologic diseases [19]-[34] is perhaps most immediately significant in providing a facile marker to identify the daughter progeny of repeated, antigen-driven proliferation cycles. However, certain other characteristics of the CD4+CD28null cells, seen here in greatest proportions among the IPF patients destined for poor outcomes, may have potential pathogenic significance.

The overall cytokine production profile of the IPF CD4+CD28null cells is highly comparable to that previously observed among analogous lymphocytes derived from distinctly different patient populations with other chronic immunologic disorders [20], [24]. Thus, it seems most likely that the singular characteristics and functional modifications of this cell phenotype are a biologically conserved (and presumably important) component of the adaptive immune response to chronic stimulation by diverse antigen(s). As such, and amongst other possibilities, the ability to generate CD4+CD28null cells with repeated/chronic antigen engagements could conceivably represent an evolutionary adaptation to more effectively counter difficult-to-eradicate microbial pathogens [19].

Chronic adaptive immune responses in human diseases are extraordinarily complicated. Antigen-activated T-cells can, in appropriate circumstances, undergo innumerable divisions, resulting in prodigious numbers of clonal daughter progeny, including variable proportions of CD4+CD28null cells [19]. In turn, these complex and changing subpopulations of T-cells with evolving phenotypes and functions produce diverse mediators that also activate and/or recruit successive waves of other immune effectors (macrophages, neutrophils, dendritic cells, etc.), as well as epithelium and many mesenchymal cells. These second- and third-tier effector cells also add their productions of interrelated, interactive, and often redundant mediators and other elaborations in what can become an unfathomably complex inflammatory conflagration (18).

Accordingly, the exact role(s) the singular functions of CD4+CD28null could directly contribute to tissue injuries of IPF (or other chronic immunologic diseases [19]-[34]) in situ cannot be deduced from highly reductionist in vitro studies of single isolated cell types. Nonetheless, it may still be notable that several TH1 and other pro-inflammatory mediators which initiate or amplify immune responses (e.g., IL-1β, IL-6, TNF-α, G-CSF) were comparatively over-produced by the IPF CD4+CD28null cells relative to their autologous CD4+CD28+ counterparts, both constitutively, and with TCR stimulation (FIG. 16). Moreover, the perforin and granzyme B productions (FIG. 15B) of the CD4+CD28null lymphocytes could conceivably account for infiltrates of cytotoxic granule-laden lymphocytes that are present in IPF lungs [40] (FIG. 20), and are believed to contribute to the pulmonary epithelial apoptosis of this disease [35]. In comparison, TH2 cytokine productions by the CD4+CD28null cells were typically relatively little increased upon TCR stimulation, although their elaboration of IL-4, a potentially important mediator of fibrogenesis [41], is a notable exception.

Furthermore, the many pro-injurious mediator productions of CD4+CD28null cells are also strikingly contrasted with their relative deficiencies of opposing, counter-regulatory functions that could potentially limit or modify inflammatory processes. The comparatively lesser production of IL-10 by CD4+CD28null cells may have particular biologic importance in fibrotic disorders, given the possible roles of this cytokine in general suppression of deleterious immune responses [42] and inhibition of TGF-β-mediated fibrogenesis [43]. CD28 signaling has been implicated in the induction of FoxP3 and generation of CD4+CD25+Tregs [44]. Consequently, lack of this costimulatory function could plausibly account for the comparative paucity of FoxP3 among the CD4+CD28null cells, and these findings are also broadly concordant with recent observations that CD4+FoxP3+Tregs are generally diminished among IPF patients (17). The net effects of the seeming imbalance between pro-injurious and counter-regulatory (dampening) functions could conceivably result in promotion of more injurious responses with serial (chronic) antigen engagements (including intrapulmonary antigens [13]) by T-lymphocyte populations that have increased proportions of CD4+CD28null cells.

Because of the downstream amplification of T-cell effector responses [1,8], and extraordinary numbers of T-cells in an individual, antigen activation of even a limited fraction of total lymphocytes can have far-reaching effects. The proportion of peripheral CD4+CD28null cells among the CD28% Low subjects here was ˜34% (Table 1). Since the circulating T-cell compartment represents only a miniscule fraction of the lymphocytes that traffic from inflamed tissue and proximate lymph nodes [45], [46], the total numbers of these highly altered lymphocytes in the IPF patients is incalculably large. By comparison, the precursor frequency for T-cells reactive to conventional peptide antigens (e.g., influenza epitopes) is estimated at 1:1000 to 1:10,000 [47]. Microbial superantigens are among the most potent antigens known, can mediate life-threatening toxic shock syndrome, and activate ˜−20% of total T-cells among infected individuals [48]. Alloantigens are often highly immunogenic, capable of evoking brisk, severe, and refractory allograft rejection, and are initially recognized by ≦10% of the recipient T-cell repertoire [49]. Daughter progeny of T-cell clonal proliferations (which can develop into CD4+CD28null cells [19]) comprise a very similar proportion of circulating CD4 T-cells (˜36%) among lung transplant recipients with severe lung damage due to obliterative bronchiolitis [50]. The frequency of CD4+CD28null cells among the IPF patients destined for poor outcomes here are also highly comparable to the proportions of these cells among lung transplant recipients with chronic rejection who had similarly guarded prognoses [20], and is even greater than in many other disease conditions wherein these particular lymphocytes have been implicated in pathogenesis [21]-[34]. Thus, the CD4+CD28null cells among many of the IPF patients here appear at least numerous enough to be potentially capable of causing or contributing to disease manifestations.

This initial, cross-sectional, exploratory examination was focused on investigating potential immunopathogenic phenomena of IPF per se and, as such, excluded subjects with infections, malignancies, accelerated IPF, or co-morbid autoimmune disorders that could, perhaps, confound the operating characteristics of these T-cell assays in actual patient management.

In summary, the present data show that the extent of CD28 expression on circulating CD4 T-cells of IPF patients is associated with disease outcomes. Among other implications, these findings are further evidence for a role of adaptive immune processes in the pathogenesis of IPF [3]-[15], [17]. The practical value of simple, minimally invasive T-cell assays that identify patients destined for disease progression would be considerable. Thus, heightened surveillance and/or earlier interventions (e.g., lung transplantations or experimental therapies) could be directed to those patients at greatest immediate risk, while possibly obviating morbid treatments in those destined for more indolent courses.

### 14. REFERENCES

1. American Thoracic Society (2000) Idiopathic pulmonary fibrosis: diagnosis and treatment. International consensus statement. American Thoracic Society (ATS), and the European Respiratory Society (ERS). Am J Respir Crit. Care Med 161: 646-664.
2. Selman M, Thannickal V J, Pardo A, Zisman D A, Martinez F J, et al. (2004) Idiopathic pulmonary fibrosis: pathogenesis and therapeutic approaches. Drugs 64: 406-430.
3. Kurosu K, Takiguchi Y, Okada O, Yumoto N, Sakao S, et al. (2008) Identification of annexin 1 as a novel autoantigen in acute exacerbation of idiopathic pulmonary fibrosis. J Immunol 181: 756-767.
4. Takahashi T, Wada I, Ohtsuka Y, Munakata M, Homm Y, et al. (2007) Autoantibody to alanyl-tRNA synthetase in patients with idiopathic pulmonary fibrosis. Respirology 12: 642-653.
5. Magro C M, Waldman W J, Knight D A, Allen J N, Nadasdy T, et al. (2006) Idiopathic pulmonary fibrosis related to endothelial injury and antiendothelial cell antibodies. Hum Immunol 67: 284-297.
6. Dobashi N, Fujita J, Murota M, Ohtsuki Y, Yamadori I, et al. (2000) Elevation of anti-cytokeratin 18 antibody and circulating cytokeratin 18: anti-cytokeratin 18 antibody immune complexes in sera of patients with idiopathic pulmonary fibrosis. Lung 178: 171-179.
7. Wallace W A, Schofield J A, Lamb D, Howie S E (1994) Localization of a pulmonary autoantigen in cryptogenic fibrosing alveolitis. Thorax 49: 1139-1145.
8. Pignatti P, Brunetti G, Moretto D, Yacoub M R, Fiori M, et al. (2006) Role of the chemokine receptors CXCR3 and CCR4 in human pulmonary fibrosis. Am J Respir Crit Care Med 173: 310-317.
9. Papiris S A, Kollintza A, Karatza M, Manali E D, Sotiropoulou C, et al. (2007) CD8+ T lymphocytes in broncholaveolar lavage in idiopathic pulmonary fibrosis. J Inflamm (Lond) 4: 14-18.
10. Marchal-Somme J, Uzunhan Y, Marchand-Adam S, Valeyre D, Soumelis V, et al. (2006) Cutting edge: non-proliferating mature immune cells form a novel type of organizing lymphoid structure in idiopathic pulmonary fibrosis. J Immunol 176: 5735-5739.
11. Homolka J, Ziegenhagen M W, Gaede K I, Entzian P, Zissel G, et al. (2003) Systemic immune cell activation in a subgroup of patients with idiopathic pulmonary fibrosis. Respiration 70: 262-269.
12. Shimizudani A, Murata H, Keino H, Kojo S, Nakamura H, et al. (2002) Conserved CDR 3 region of T cell receptor BV gene in lymphocytes from bronchoalveolar lavage fluid of patients with idiopathic pulmonary fibrosis. Clin Exp Immunol 129: 140-149.
13. Feghali-Bostwick C A, Tsai C G, Valentine V G, Kantrow S, Stoner M W, et al. (2007) Cellular and humoral autoreactivity in idiopathic pulmonary fibrosis. J Immunol 179: 2592-9.
14. Rosas I O, Ren P, Avila N A, Chow C K, Franks T J, et al. (2007) Early interstitial lung disease in familial pulmonary fibrosis. Am J Resp Crit. Care Med 176: 698-705.
15. Marchal-Somme J, Uzunhan Y, Marchand-Adam S, Kambouchner K, Valeyre D, et al. (2007) Dendritic cells accumulate in human fibrotic interstitial lung disease. Am J Respir Crit Care Med 176: 1007-1014.
16. Sakaguchi S (2004) Naturally arising CD4+ regulatory t cells for immunologic self-tolerance and negative control of immune responses. Annu Rev Immunol 22: 531-562.
17. Kotslanidis I, Nakou E, Bouchliou I, Tzouvelekis A, Spanoudakis E, et al. (2009) Global impairment of CD4+CD25+FoxP3+ regulatory T cells in idiopathic pulmonary fibrosis. Am J Resp Crit Care Med 179: 1121-1130.
18. Monaco C, Andreakos E, Kiriakidis S, Feldman M, Paleolog C (2004) T-cell-mediated signaling in immune, inflammatory and angiogenic processes: the cascade of events leading to inflammatory diseases. Curr Drug Targets Inflamm Allergy 3: 35-42.
19. Vallejo A N, Weyand C M, Goronzy J J (2004) T-cell senescence: a culprit of immune abnormalities in chronic inflammation and persistent infection. Trends Mol Med 10: 119-124.
20. Studer S M, George M P, Zhu X, Song Y, Valentine V G, et al. (2008) CD28 Down-regulation on CD4 T-cells is a marker for graft dysfunction in lung transplant recipients. Am J Resp Crit Care Med 178: 765-773.
21. Liuzzo G, Goronzy J J, Yang H, Kopecky S L, Holmes D R, et al. (2000) Monoclonal T-cell proliferation and plaque instability in acute coronary syndromes. Circulation 101: 2883-2888.
22. Martens P B, Goronzy J J, Schaid D, Weyand C M (1997) Expansion of unusual CD4+ T cells in severe rheumatoid arthritis. Arth and Rhem 40: 1106-14.
23. Goronzy J J, Matteson E L, Fulbright J W, Warrington K J, Chang-Miller A, et al. (2004) Prognostic markers of radiographic progression in early rheumatoid arthritis. Arthritis Rheum 50: 43-54.
24. Komocsi A, Lamprecht P, Csernok E, Mueller A, Holl-Ulrcih K, et al. (2002) Peripheral blood and granuloma CD4+CD28− T cells are a major source of interferon-gamma and tumor necrosis factor-alpha in Wegener's granulomatosis. Am J Pathol 160: 1717-1724.
25. Colombatti A, Doliana R, Schiappacassi M, Argentini C, Tonutti E, et al. (1998) Age-related persistent clonal expansions of CD28− cells: phenotypic and molecular TCR analysis reveals both CD4+ and CD4+CD8+ cells with identical CDR3 sequences. Clin Immunol Immunopath 89: 61-70.
26. Nakajima T, Schulte S, Warrington K J, Kopecky S L, Frye R L, et al. (2002) T-cell-mediated lysis of endothelial cells in acute coronary syndromes. Circulation 105: 570-575.
27. Hirokawa M, Horiuchi T, Kawabata Y, Kitabayashi A, Saitoh H, et al. (2001) Oligoclonal expansion of CD4+CD28− T lymphocytes in recipients of allogeneic hematopoietic cell grafts and identification of the same T cell clones within both CD4+CD28+ and CD4+CD28− T cell subsets. Bone Marrow Transpl 27: 1095-1100.
28. Zal B, Kaski J C, Arno G, Akiyu J P, Xu Z, et al. (2004) Heat-shock protein 60-reactive CD4+CD28null T cells in patients with acute coronary syndromes. Circulation 109(10): 1230-1235.
29. Schmidt D, Goronzy J J, Weyand C M (1996) CD4+ CD7− CD28− T-cells are expanded in rheumatoid arthritis and are characterized by autoreactivity. J Clin Invest 97: 2027-2037.
30. Jendro M C, Ganten T, Matteson E L, Weyand C M, Goronzy J J (1995) Emergence of oligoclonal T cell populations following therapeutic T cell depletion in rheumatoid arthritis. Arthritis Rheum 38: 1242-1251.
31. Schirmer M, Vallejo A N, Weyand C M, Goronzy J J (1998) Resistance to apoptosis and elevated expression of Bcl-2 in clonally expanded CD4+CD28− T cells from rheumatoid arthritis patients. J Immunol 161: 1018-1025.
32. Fasth A E R, Dstmalchi M, Rahbar A, Salomonsson S, Pandya J M, et al. (2009) T cell infiltrates in the muscles of patients with dermatomyositis and polymyositis are dominated by CD28null T cells. J Immunol 183: 4792-4799.
33. Lambers C, Hacker S, Posch M, Hoetzenecker K, Pollreisz A, et al. (2009) T cell senescence and contraction of T cell repertoire diversity in patients with chronic obstructive pulmonary disease. Clin Exp Immunol 155(3): 466-475.
34. Duffner C, Seiler R, Klein-Weigel H, Gobel C, Goldberger C, et al. (2005) High prevalence of circulating CD4+CD28− T-cells in patients with small abdominal aortic aneurysms. Arterioscler Thromb Vasc Biol 25: 1347-1352.
35. Barbos-Filho J V, Ferreira M A, Sesso A, Kairalla R A, Carvalho C R, et al. (2001) Evidence of type H pneumocyte apoptosis in the pathogenesis of idiopathic pulmonary fibrosis (IPF)/usual interstitial pneumonia (UIP). J Clin Pathol 54: 132-138.
36. Mason D P, Brizzio M E, Alster J M, McNeill A M, Murthy S C, et al. (2007) Lung transplantation for idiopathic pulmonary fibrosis. Ann Thorac Surg 84: 1121-1128.
37. Collard H R, King T E, Bartelson B B, Vourlekis J S, Schwartz M I, et al. (2004) Changes in clinical and physiologic variable predict survival in idiopathic pulmonary fibrosis. Am J Respir Crit. Care Med 169: 538-542.
38. Sharpe A H, Freeman G J (2002) The B7-CD28 superfamily. Nat Rev Immunol 2: 116-126.
39. Fontenot A P, Gharavi L, Bennett S R, Canavera S J, Newman L S, et al. (2003) CD28 costimulation independence of target organ versus circulating memory antigen-specific CD4 T cells. J Clin Invest 112: 776-784.
40. Miyazaki H, Kuwano K, Yoshida K, Maeyama T, Yoshimi M, et al. (2004) The perforin mediated apoptotic pathway in lung injury and fibrosis. J Clin Path 57: 1292-1298.
41. Atamas S P, Luzina I G, Dai H, Wilt S G, White B (2002) Synergy between CD40 ligation and IL-4 on fibroblast proliferation involves IL-4 receptor signaling. J Immunol 168: 1139-1145.
42. Conti P, Kempuraj D, Kandere K, Di Gioacchino M, Barbaca R C, et al. (2003) IL-10, an inflammatory/inhibitory cytokine, but not always. Immunol Lett 86: 123-129.
Kitani A, Fuss I, Nakamura I K, Kumaki F, Usui T, et al. (2003) Transforming growth factor (TGF)-β1 producing regulatory T-cells induce Smad-mediated interleukin 10 secretion that facilitates coordinated immunoregulatory activity and amelioration of TGF-β1-mediated fibrosis. J Exp Med 198: 1179-1188.
44. Tai X, Cowan M, Feigenbaum I, Singer A (2005) CD28 costimulation of developing thymocytes induces FoxP3 expression and regulatory T cell differentiation independently of interleukin 2. Nat Immunol 6: 152-156.
45. Westermann J, Pabst R (1992) Distribution of lymphocyte subsets and natural killer cells in the human body. Clin Investig 70: 539-544.
46. Sallusto F, Lanzaveechia A (2000) Understanding dendritic cell and T-lymphocyte traffic through the analysis of chemokine receptor expression. Immunol Rev 177: 134-40.
47. Scheibenbogen C, Romero P, Rivoltini L, Herr W, Schmittel A, et al. (2000) Quantitation of antigen-reactive T cells in peripheral blood by IFNgamma-ELISPOT assay and chromium-release assay: a four-centre comparative trial. J Immunol Methods 244(1-2): 81-9.
48. Fraser J D, Proft T. The bacterial superantigen and superantigen-like proteins. Immunol Rev 2008; 225: 226-243.
49. Duncan S R, Leonard C, Theodore J, Lega M, Girgis R E, et al. (2002) Oligoclonal CD4+ T-Cell Expansions in Lung Transplant Recipients with Obliterative Bronchioloitis. Am J Respir Crit Care Med 165: 1439-46.
50. Krensky A M, Weiss A, Crabtreee G, Davis M M, Parham P (1990) T-lymphocyte-antigen interactions in transplant recipients. N Eng J Med 322: 510-517.
51. Katzenstein A-L, Myers J L (1998) Idiopathic pulmonary fibrosis. Clinical relevance of pathologic classification. Am J Respir Crit Care Med 157: 1301-1315.
52. Zhu X, Gadgil A S, Givelber R, George M P, Stoner M W, et al. (2009) Peripheral T-cell functions correlate with the severity of chronic obstructive pulmonary disease. J Immunol 182: 3270-3277.

Various publications are cited herein, the contents of which are hereby incorporated by reference in their entireties.

What is claimed is:

1. A method of determining the risk that a subject suffering from a chronic pulmonary disease will suffer a severe adverse event, comprising determining whether, in a sample comprising T-cells collected from the subject, one or more of the following is present:
the proportion of CD4+CD28null cells among the Cd4+ T-cell population is elevated, where said elevation indicates that the subject is at increased risk of suffering a severe adverse event;
the proportion of CD4+ cells among the peripheral blood mononuclear cell population is decreased, where said decrease indicates that the subject is at increased risk of suffering a severe adverse event;
the proportion of CD25+ cells among the CD4+ T-cell population is decreased, where said decrease indicates that the subject is at increased risk of suffering a severe adverse event;
the proportion of CD28+ cells among the CD8+ T-cell population is decreased, where said decrease indicates that the subject is at increased risk of suffering a severe adverse event;
the proportion of CD4+CD45+RO CD45RO+ cells among the CD4+ T-cell population is decreased, where said decrease indicates that the subject is at increased risk of suffering a severe adverse event;
the proportion of HLA-DR+ cells among the CD4+ T-cell population is increased, where said increase indicates that the subject is at increased risk of suffering a severe adverse event;
the proportion of CD27+ cells among the CD4+ T-cell population is decreased relative to a healthy control, where said decrease indicates that the subject is at increased risk of suffering an adverse event;
the proportion of CCR7+ cells among the CD4+ T-cell population is decreased relative to a healthy control, where said decrease indicates that the subject is at increased risk of suffering an adverse event;
the proportion of IL-7R+ cells among the CD4+ T-cell population is decreased relative to a healthy control, where said decrease indicates the subject is at increased risk of suffering an adverse event;
the proportion of CD3+ cells among the CD4+ T-cell population is decreased relative to a healthy control, where said decrease indicates the subject is at increased risk of suffering an adverse event;
the proportion of CD80+ cells among the CD4+ T-cell population is increased relative to a healthy control, where said decrease indicates the subject is at increased risk of suffering an adverse event;
the proportion of CTLA-4+ cells among the CD4+ T-cell population is decreased relative to a healthy control, where said decrease indicates the subject is at increased risk of suffering an adverse event;
the proportion of ICOS+ cells among the CD4+ T-cell population is decreased relative to a healthy control, where said decrease indicates the subject is at increased risk of suffering an adverse event;
the proportion of Itk+ cells among the CD4+ T-cell population is decreased relative to a healthy control, where said decrease indicates the subject is at increased risk of suffering an adverse event;
the proportion of Ltk+ cells among the CD4+ T-cell population is decreased relative to a healthy control, where said decrease indicates the subject is at increased risk of suffering an adverse event;
the proportion of granzyme B produced by CD4+ T-cells of the subject is increased relative to T-cells of a healthy control, where said increase indicates that the subject is at increased risk of suffering an adverse event;
the proportion of FoxP3 produced by CD4+ T cells of the subject is decreased relative to T cells of a healthy control, where said decrease indicates that the subject is at increased risk of suffering an adverse event; and
the proportion of perforin produced by CD4+ T-cells of the subject is increased relative to T-cells of a healthy control, where said increase indicates that the subject is at increased risk of suffering an adverse event.

2. The method of claim 1, where the chronic pulmonary disease is selected from the group consisting of chronic rejection following lung transplant, idiopathic pulmonary fibrosis, chronic obstructive pulmonary disease, cystic fibrosis, pulmonary hypertension, inflammatory lung disease associated with an autoimmune disease, and lung disease associated with sarcoidosis and scleroderma.

3. The method of claim 1, where the chronic pulmonary disease is idiopathic pulmonary fibrosis.

4. The method of claim 1, where the chronic pulmonary disease is chronic rejection following lung transplant.

5. The method of claim 1, where the adverse event is selected from the group consisting of death within one year, lung transplant desirable or deemed necessary within one year, (in transplant patients) incidence of BOS, resistance to therapy with cyclosporine or other immunosuppressive agent, decrease in DLCO, or decrease in FVC, or decrease in FEV1

6. The method of claim 1, where if the percent of CD4+CD28+ of the CD4+ T-cell population is less than about 90 percent, the subject is at increased risk of suffering a severe adverse event.

7. The method of claim 1, where if the percent of CD4+ cells in the T cell population is less or equal to about 31.1 percent, the subject is at an increased risk of suffering a severe adverse event.

8. The method of claim 1, where if the percent of CD25+ cells in the T cell population is less than or equal to about 54 percent, then subject is at an increased risk of suffering a severe adverse event.

9. The method of claim 1, where if the percent of CD8+CD28+ cells in the CD8+ T cell population is less than or equal to about 21 percent, then subject is at an increased risk of suffering a severe adverse event.

10. The method of claim 1, where if the percent of CD45RO+ cells in the CD4+ T cell population is less than or equal to about 54 percent, then subject is at an increased risk of suffering a severe adverse event.

11. The method of claim 1, where if the percent of CD4+HLA−DR+ cells in the CD4+ T cell population is greater than about 87.1 percent, then subject is at an increased risk of suffering a severe adverse event.

12. A kit comprising a capture agent directed to one or more of CD4, CD28, CD25, CD8, CD45, DR, Ro, CD27, CCR7, granzyme B, FoxP3, IL-7R, or perforin, optionally together with a detection agent, and optionally with a package insert describing one or more method according to claim 1.
